(19) United States
(12) Patent Application Publication
(10) Pub. No.: US 2026/0255873 A1
(43) Pub. Date: Aug. 27, 2026

(54) ORGANIC ELECTROLUMINESCENT ELEMENT

(71) Applicants: NIPPON STEEL CHEMICAL & MATERIAL CO., LTD., Tokyo (JP); Kyushu University, National University Corporation, Fukuoka-shi, Fukuoka (JP)

(72) Inventors: Munetomo INOUE, Tokyo (JP); Yuta SAGARA, Tokyo (JP); Takuma YASUDA, Fukuoka-shi, Fukuoka (JP)

(73) Assignees: NIPPON STEEL CHEMICAL & MATERIAL CO., LTD., Tokyo (JP); Kyushu University, National University Corporation, Fukuoka-shi, Fukuoka (JP)

(21) Appl. No.: 19/161,726

(22) PCT Filed: Mar. 18, 2024
(86) PCT No.: PCT/JP2024/010399

§ 371 Date: Sep. 3, 2025

(30) Foreign Application Priority Data
Mar. 30, 2023 (JP) 2023-055438

(51) Int. Cl. H10K 85/60; C07D 403/14; C09K 11/02; C09K 11/06; H10K 50/12

(52) U.S. Cl. CPC H10K 85/654; C07D 403/14; C09K 11/02; C09K 11/06; H10K 85/636; H10K 85/6572; C09K 2211/1007; C09K 2211/1018; H10K 50/12; H10K 85/658

(57) ABSTRACT

An organic EL device uses a compound represented by a general formula and can be an organic EL device having high emission efficiency and a long lifetime. The compound for an organic electroluminescent device may be represented by the general formula, with, Ad being an adamantyl group represented by another general formula. X independently represents N or CR¹, and at least one X represents N. R¹ independently represents hydrogen and the like. Ar¹ represents a substituted or unsubstituted aromatic hydrocarbon group having 6 to 18 carbon atoms and the like. R independently represents a substituted or unsubstituted aromatic heterocyclic group having 3 to 17 carbon atoms and the like. L¹ and L² each independently represent a direct bond and the like. “a” to f represent a number of substitutions and independently represent an integer of 0 to 4, where b+c+d+e+f≥1.

### TECHNICAL FIELD

The present invention relates to a compound, a material for organic electroluminescent device, as well as an organic electroluminescent device or element (also referred to as an organic EL device or element).

When a voltage is applied to an organic EL device, holes and electrons are injected from the anode and the cathode, respectively, into the light emitting layer. Then, the injected holes and electrons are recombined in the light emitting layer to thereby generate excitons. At this time, according to the electron spin statistics theory, singlet excitons and triplet excitons are generated at a ratio of 1:3. In the fluorescent organic EL device that uses emission caused by singlet excitons, the limit of the internal quantum efficiency is said to be 25%. On the other hand, it has been known that, in the phosphorescent organic EL device that uses emission caused by triplet excitons, the internal quantum efficiency can be enhanced up to 100% when intersystem crossing efficiently occurs from singlet excitons. However, the phosphorescent organic EL device has a technical problem of further extending the lifetime.

Further, a highly efficient organic EL device utilizing delayed fluorescence has been developed, in recent years. For example, Patent Literature 1 discloses an organic EL device utilizing the Triplet-Triplet Fusion (TTF) mechanism, which is one of the mechanisms of delayed fluorescence. The TTF mechanism utilizes a phenomenon in which a singlet exciton is generated by the collision of two triplet excitons, and it is believed that the internal quantum efficiency can be enhanced up to 40%, in theory. However, its efficiency is low as compared with the efficiency of the phosphorescent organic EL device, and thus further improvement in efficiency is desired.

On the other hand, Patent Literature 2 discloses an organic EL device utilizing the Thermally Activated Delayed Fluorescence (TADF) mechanism. The TADF mechanism utilizes a phenomenon in which reverse intersystem crossing occurs from the triplet exciton to the singlet exciton in a material having a small energy difference between the singlet level and the triplet level, and it is believed that the internal quantum efficiency can be enhanced up to 100%, in theory. However, further improvement in lifetime characteristics is desired as in the phosphorescent device, and for a blue light emitting organic EL device, improvement in lifetime characteristics is particularly demanded. More specifically, polycyclic aromatic compounds that exhibit blue light emission by utilizing the TADF mechanism have low resistance to holes and electrons, making it difficult to ensure a practical device lifetime when used in an organic EL device in combination with a conventional known host, as a result of which improvement in lifetime characteristics including not only improvement in a dopant but also development on a host material that is highly resistant to holes and electrons, is demanded.

### CITATION LIST

### Patent Literature

Patent Literature 1: WO2010/134350
Patent Literature 2: WO2011/070963
Patent Literature 3: WO2015/102118
Patent Literature 4: WO2017/115833
Patent Literature 5: WO2018/212169
Patent Literature 6: WO2018/181188
Patent Literature 7: WO2020/040298
Patent Literature 8: JP2020-120096 A
Patent Literature 9: WO2008/117826
Patent Literature 10: CN112778278
Patent Literature 11: WO2022/027992
Patent Literature 12: WO2021/228111

Patent Literature 4 discloses an organic EL device containing two host materials exemplified by compounds shown below, and a TADF material as light emitting dopants in a light emitting layer.

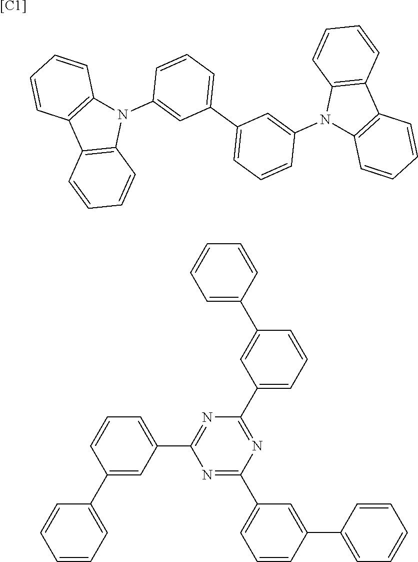

Patent Literature 3 and Patent Literature 5 disclose an organic EL device in which a TADF material including a polycyclic aromatic compound exemplified by the following compound is used as a light emitting dopant.

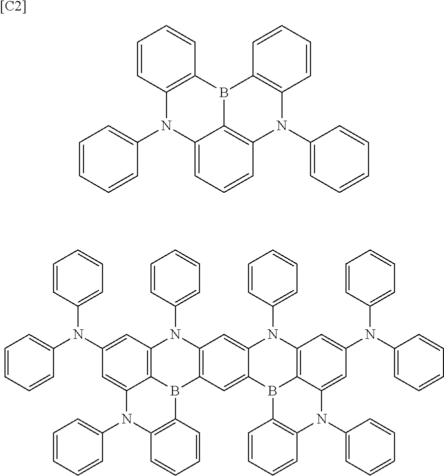

Patent Literatures 6 and 7 disclose an organic EL device in which a mixture of a boron-based compound, a TADF material, and the following carbazole compound, is used in a light emitting layer.

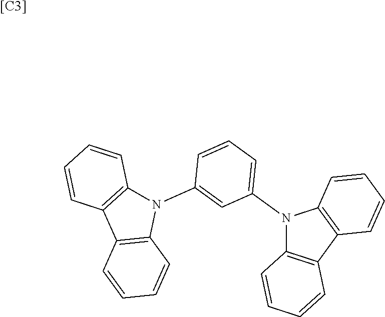

Patent Literature 8 discloses an organic EL device in which a mixture of the following boron-based compound a7, nitrogen-containing six-membered ring compound a8, and carbazole compound a9, is used in a light-emitting layer.

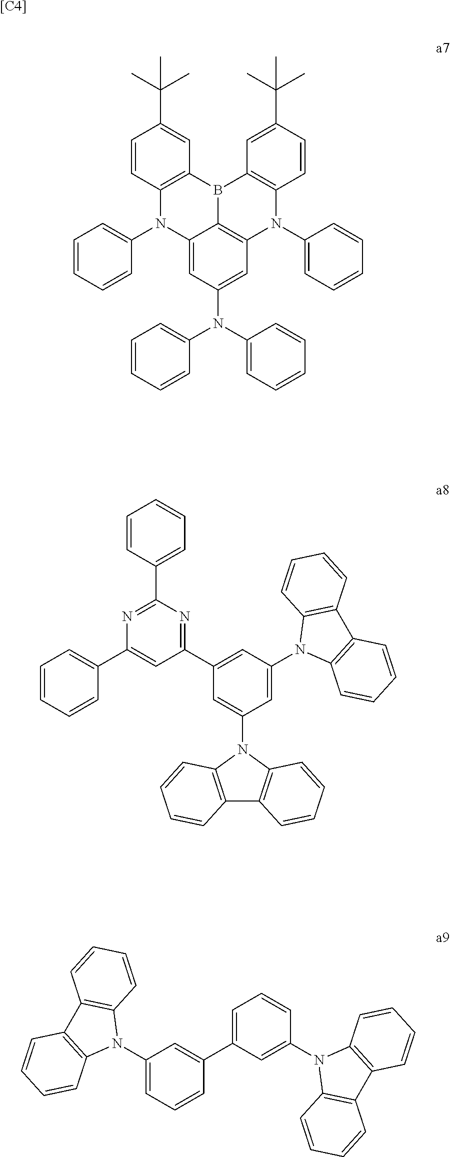

Patent Literature 9 discloses a phosphorescent organic EL device in which a compound having a nitrogen-containing six-membered ring and carbazole linked to each other and exemplified by the following compound is used as a host material.

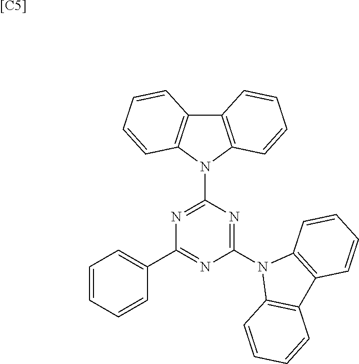

Patent Literature 10 discloses an organic EL device in which a compound having a backbone of a nitrogen-containing six-membered ring and carbazole linked to each other, which is further linked with an adamantyl group, and exemplified by the compound below is used as a host material.

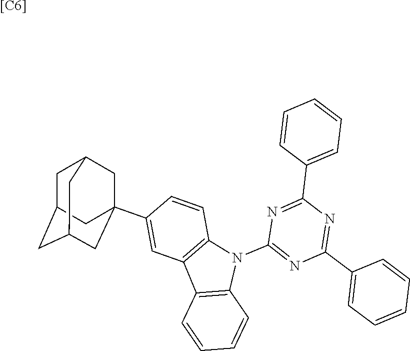

Patent Literature 11 discloses an organic EL device in which a compound having a nitrogen-containing six-membered ring linked with a cyano group and an adamantyl group and exemplified by the compound below is used as a host material.

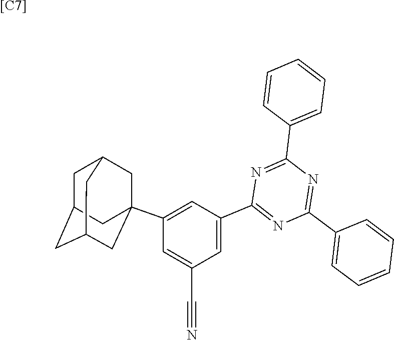

Patent Literature 12 discloses an organic EL device in which a compound having dibenzofuran and an adamantyl group linked to each other and exemplified by the compound below is used as a host material

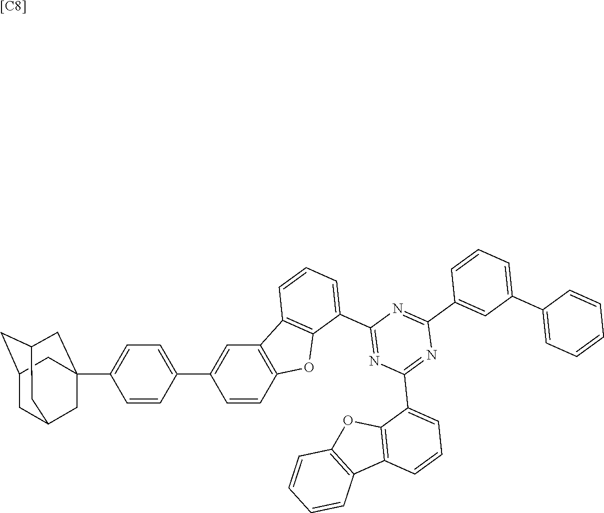

In all the patent literatures, however, there is still room for improvement in organic EL devices that exhibit sufficient lifetime characteristics.

### SUMMARY OF INVENTION

### Technical Problem

In order to apply an organic EL device to a display device or a light source for a flat panel display and the like, it is necessary to improve emission efficiency of the device and simultaneously to ensure sufficient stability upon driving. An object of the present invention is to provide a practically useful organic EL device that has high efficiency and long lifetime characteristics, and a compound suitable for the device.

### Solution to Problem

The present invention relates to a material for an organic electroluminescent device, composed of a compound represented by the following general formula (1):

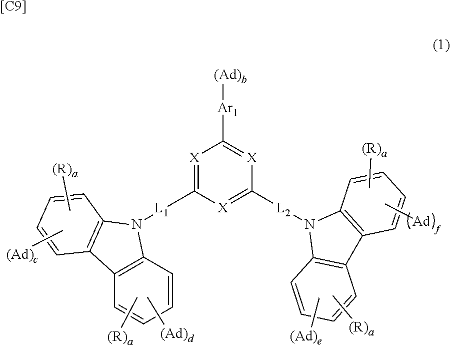

wherein Ad is an adamantyl group represented by the following general formula (2), and is preferably represented by the following general formula (3):

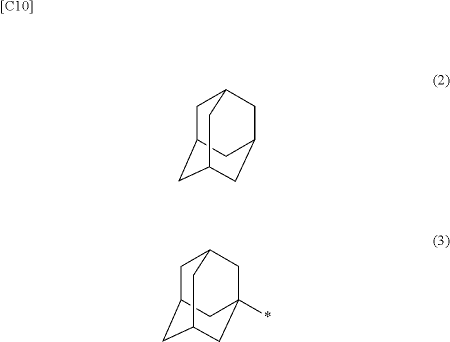

wherein “*” is a bonding site to the general formula (1) above.

X independently represents N or CR¹ and at least one X represents N, and preferably each X represents N.

R¹ independently represents hydrogen, deuterium, an aliphatic hydrocarbon group having 1 to 10 carbon atoms, a substituted or unsubstituted aromatic hydrocarbon group having 6 to 18 carbon atoms, a substituted or unsubstituted aromatic heterocyclic group having 3 to 17 carbon atoms, or a substituted or unsubstituted linked aromatic group formed by linking 2 to 8 of these aromatic groups.

Ar¹ represents a substituted or unsubstituted aromatic hydrocarbon group having 6 to 18 carbon atoms, a substituted or unsubstituted aromatic heterocyclic group having 3 to 17 carbon atoms, or a substituted or unsubstituted linked aromatic group formed by linking 2 to 8 of these aromatic groups, and Ar¹ preferably represents a substituted or unsubstituted aromatic hydrocarbon group having 6 to 11 carbon atoms, a substituted or unsubstituted aromatic heterocyclic group having 3 to 11 carbon atoms, or a substituted or unsubstituted linked aromatic group formed by linking 2 to 8 of these aromatic groups. Ar¹ more preferably represents a substituted or unsubstituted aromatic hydrocarbon group having 6 to 11 carbon atoms, or a substituted or unsubstituted linked aromatic group formed by linking 2 to 5 of these aromatic groups.

R independently represents deuterium, an aliphatic hydrocarbon group having 1 to 10 carbon atoms, a substituted or unsubstituted aromatic hydrocarbon group having 6 to 18 carbon atoms, or a substituted or unsubstituted aromatic heterocyclic group having 3 to 17 carbon atoms.

L¹ and L² each independently represent a direct bond, a substituted or unsubstituted aromatic hydrocarbon group having 6 to 18 carbon atoms, a substituted or unsubstituted aromatic heterocyclic group having 3 to 17 carbon atoms, or a substituted or unsubstituted linked aromatic group formed by linking 2 to 8 of these aromatic groups; L¹ and L² preferably each independently represent a direct bond, a substituted or unsubstituted aromatic hydrocarbon group having 6 to 18 carbon atoms, or a substituted or unsubstituted aromatic heterocyclic group having 3 to 17 carbon atoms, with the direct bond being more preferred.

a represents the number of substitutions and independently represents an integer of 0 to 4, and preferably each a represents 0. b to f represent the number of substitutions and independently represent an integer of 0 to 4. Here, b+c+d+e+f≥1 is satisfied, preferably b+c+d≥1, and more preferably b+c+d+e+f≥2.

The compound for an organic electroluminescent device of the present invention represented by the general formula (1) above preferably has a glass transition temperature (Tg) of 135° C. or higher and more preferably 140° C. or higher.

The organic electroluminescent device of the present invention is an organic electroluminescent device that includes one or more light emitting layers between an anode and a cathode opposite to each other, wherein at least one of the light emitting layers preferably contains a host selected from the compounds represented by the general formula (1) above, and a light emitting dopant containing a boron atom.

Also the light emitting dopant is preferably a light emitting dopant selected from a polycyclic aromatic compound represented by the following general formula (4a) or (4b):

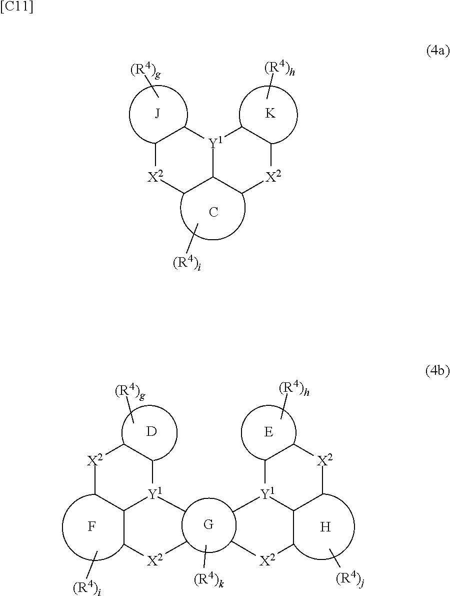

Here, a ring J, a ring K, a ring C, a ring D, a ring E, a ring F, a ring G, and a ring H are each independently a substituted or unsubstituted aromatic hydrocarbon ring having 6 to 24 carbon atoms, or a substituted or unsubstituted aromatic heterocyclic ring having 3 to 17 carbon atoms.

Y¹ is each independently B, P, P═O, P═S, Al, Ga, As, Si—R³ or Ge—R³, preferably B, P, P═O or P═S, and more preferably B.

R³ is each independently an aliphatic hydrocarbon group having 1 to 10 carbon atoms, a substituted or unsubstituted aromatic hydrocarbon group having 6 to 18 carbon atoms, or a substituted or unsubstituted aromatic heterocyclic group having 3 to 17 carbon atoms.

X² is each independently O, N—Ar⁴, S or Se, preferably O, N—Ar⁴ or S, and more preferably O or N—Ar⁴.

Ar⁴ is each independently a substituted or unsubstituted aromatic hydrocarbon group having 6 to 18 carbon atoms, a substituted or unsubstituted aromatic heterocyclic group having 3 to 17 carbon atoms, or a substituted or unsubstituted linked aromatic group formed by linking 2 to 8 of these groups.

N—Ar⁴ is optionally bonded to any of the ring J, the ring K, the ring C, the ring D, the ring E, the ring F, the ring G, or the ring H to form a heterocyclic ring containing N.

R⁴ each independently represents a cyano group, deuterium, a diarylamino group having 12 to 44 carbon atoms, an arylheteroarylamino group having 12 to 44 carbon atoms, a diheteroarylamino group having 12 to 44 carbon atoms, an aliphatic hydrocarbon group having 1 to 10 carbon atoms, a substituted or unsubstituted aromatic hydrocarbon group having 6 to 18 carbon atoms, or a substituted or unsubstituted aromatic heterocyclic group having 3 to 17 carbon atoms.

g and h represent the number of substitutions and each independently represent an integer of 0 to 4, i and j represent the number of substitutions and each independently represent an integer of 0 to 3, and k represents the number of substitutions and represents an integer of 0 to 2.

A preferred example of the polycyclic aromatic compound represented by the general formula (4a) above includes a boron-containing polycyclic aromatic compound represented by the following formula (5a), and a preferred example of the polycyclic aromatic compound represented by the general formula (4b) above includes a boron-containing polycyclic aromatic compound represented by the following formula (5b).

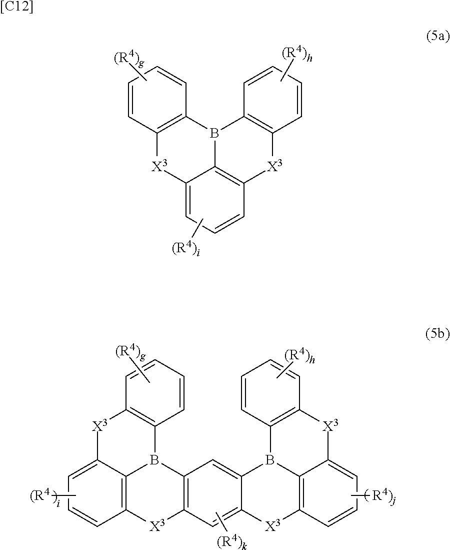

Here, X³ each independently represents N—Ar⁴, O, or S, and at least one X³ represents N—Ar⁴. Ar⁴, R⁴, g, h, i, j and k are as defined for the general formulae (4a) or (4b).

The polycyclic aromatic compounds represented by the general formulae (4a), (4b), (5a), and (5b) above preferably have a difference between excited singlet energy (S1) and excited triplet energy (T1) (AEST) as calculated by measurement of emission spectrum and a phosphorescence spectrum, of 0.20 eV or less, more preferably 0.18 eV or less and still more preferably 0.10 eV or less.

The organic electroluminescent device of the present invention preferably includes, in the organic electroluminescent device that includes one or more light emitting layers between an anode and a cathode opposite to each other, a first host selected from the compounds represented by the general formula (1) above, a second host, and a light emitting dopant containing a boron atom, and more preferably includes a compound represented by the following general formula (6) as the second host. It is also preferable that the compound represented by the general formula (1) above is an electron transporting host, and the second host is a hole transporting host.

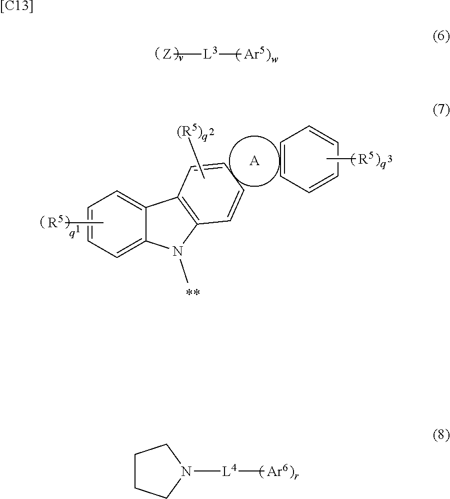

wherein Z in general formula (6) is an indolocarbazole ring-containing group represented by general formula (7), “**” represents a bonding site to L³. The ring A in general formula (7) is also a heterocyclic ring represented by general formula (8), and this ring A is condensed with the adjacent ring at arbitrary position thereof.

L³ and L⁴ are each independently a substituted or unsubstituted aromatic hydrocarbon group having 6 to 18 carbon atoms, or a substituted or unsubstituted aromatic heterocyclic group having 3 to 17 carbon atoms.

Ar⁵ and Ar⁶ are each independently deuterium, a substituted or unsubstituted aromatic hydrocarbon group having 6 to 18 carbon atoms, a substituted or unsubstituted aromatic heterocyclic group having 3 to 17 carbon atoms, or a substituted or unsubstituted linked aromatic group formed by linking 2 to 8 of these aromatic groups.

R⁵ is each independently deuterium, an aliphatic hydrocarbon group having 1 to 10 carbon atoms, a substituted or unsubstituted aromatic hydrocarbon group having 6 to 18 carbon atoms, or a substituted or unsubstituted aromatic heterocyclic group having 3 to 17 carbon atoms.

v, w, q¹, q², q³ and r each represents the number of substitutions and v represents an integer of 1 to 3, w represents an integer of 0 to 3, q¹ and q³ each independently represent an integer of 0 to 4, q² represents an integer of 0 to 2, and r represents an integer of 0 to 3.

A preferred aspect of the general formula (6) above includes the following general formula (6a) or (6b):

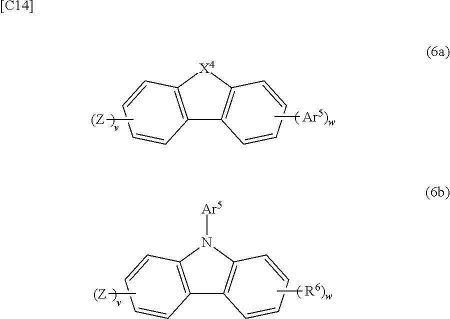

wherein Z, Ar⁵, v, and w are as defined for the general formula (6), and X⁴ represents O or S. R⁶ is each independently deuterium, an aliphatic hydrocarbon group having 1 to 10 carbon atoms, a substituted or unsubstituted aromatic hydrocarbon group having 6 to 18 carbon atoms, or a substituted or unsubstituted aromatic heterocyclic group having 3 to 17 carbon atoms.

### Advantageous Effects of Invention

The organic EL device using the compound of the present invention represented by the general formula (1) above can be an organic EL device with high emission efficiency and a long lifetime.

### BRIEF DESCRIPTION OF DRAWING

FIG. 1 shows a cross-sectional view of one example of the organic EL device.

### DESCRIPTION OF EMBODIMENTS

The present invention relates to the compound represented by the general formula (1) above used as a material for an organic electroluminescent device (organic EL device). Also, the organic EL device of the present invention includes one or more light emitting layers between an anode and a cathode opposite to each other, wherein at least one of the emitting layers contains a host selected from the compounds represented by the aforementioned general formula (1), and a light emitting dopant containing a boron atom, and preferably includes a first host selected from the compounds represented by the aforementioned general formula (1), a second host, and a light emitting dopant containing a boron atom. More preferably, at least one of the light emitting layers contains the second host selected from the compounds represented by the general formula (6) above, or alternatively contains the polycyclic aromatic compound represented by the general formula (4a) or (4b) above, specifically the polycyclic aromatic compound represented by the general formula (5a) or (5b) above, as a light emitting dopant.

First, in the present invention, the compound for an organic electroluminescent device represented by the general formula (1) above will be described. In the general formula (1) above, Ad is the adamantyl group represented by the general formula (2) above, and is preferably represented by the general formula (3) above.

X independently represents N or CR¹, at least one X represents N, and preferably each X represents N.

R¹ independently represents hydrogen, deuterium, an aliphatic hydrocarbon group having 1 to 10 carbon atoms, a substituted or unsubstituted aromatic hydrocarbon group having 6 to 18 carbon atoms, a substituted or unsubstituted aromatic heterocyclic group having 3 to 17 carbon atoms, or a substituted or unsubstituted linked aromatic group formed by linking 2 to 8 of these aromatic groups.

When R¹ is an aliphatic hydrocarbon group having 1 to 10 carbon atoms, it may be any of linear, branched, and cyclic aliphatic hydrocarbon groups, and specific examples thereof can include a linear saturated hydrocarbon group such as a methyl group, an ethyl group, a n-propyl group, a n-butyl group, a n-pentyl group, a n-hexyl group, a n-octyl group, a n-dodecyl group, a n-tetradecyl group, or a n-octadecyl group, a branched saturated hydrocarbon group such as an isopropyl group, an isobutyl group, a tert-butyl group, a neopentyl group, a 2-ethylhexyl group, or a 2-hexyloctyl group, and a saturated alicyclic hydrocarbon group such as a cyclopentyl group, a cyclohexyl group, a cyclooctyl group, a 4-butylcyclohexyl group, or a 4-dodecylcyclohexyl group. Preferably, R¹ is a methyl group, an ethyl group, a n-propyl group, a n-butyl group, a tert-butyl group, a neopentyl group, or a cyclohexyl group.

Specific examples of R¹, which is an unsubstituted aromatic hydrocarbon group having 6 to 18 carbon atoms include a group produced by removing one hydrogen atom from benzene, naphthalene, acenaphthene, acenaphthylene, anthracene, chrysene, pyrene, phenanthrene, triphenylene, fluorene, or benzo[a]anthracene. A preferred example thereof includes a group obtained from benzene, naphthalene, anthracene, chrysene, pyrene, phenanthrene, triphenylene, or fluorene. A more preferred example thereof includes a phenyl group or a naphthyl group.

Specific examples of R¹, which is an unsubstituted heteroaromatic group having 3 to 17 carbon atoms can include groups produced by removing one hydrogen from nitrogen-containing aromatic compounds having a pyrrole ring, such as pyrrole, pyrrolopyrrole, indole, isoindole, pyrroloisoindole, and carboline, or from thiophene, benzothiophene, dibenzothiophene, furan, benzofuran, dibenzofuran, carbazole, pyridine, pyrimidine, triazine, quinoline, isoquinoline, quinazoline, or quinoxaline. Preferred examples thereof include a group produced from thiophene, benzothiophene, dibenzothiophene, furan, benzofuran, dibenzofuran, or carbazole, and more preferred examples thereof include a dibenzothienyl group, a dibenzofuranyl group, or a carbazolyl group.

When R¹ is an unsubstituted linked aromatic group, specific examples thereof include a group produced by removing one hydrogen from a group formed by linking 2 to 8 aromatic groups described in the aforementioned specific examples of the unsubstituted aromatic hydrocarbon group having 6 to 18 carbon atoms and the unsubstituted heteroaromatic group having 3 to 17 carbon atoms.

Ar¹ represents a substituted or unsubstituted aromatic hydrocarbon group having 6 to 18 carbon atoms, a substituted or unsubstituted aromatic heterocyclic group having 3 to 17 carbon atoms, or a substituted or unsubstituted linked aromatic group formed by linking 2 to 8 of these aromatic groups, and Ar¹ preferably represents a substituted or unsubstituted aromatic hydrocarbon group having 6 to 11 carbon atoms, a substituted or unsubstituted aromatic heterocyclic group having 3 to 11 carbon atoms, or a substituted or unsubstituted linked aromatic group formed by linking 2 to 8 of these aromatic groups.

Specific examples of Ar¹, which is an unsubstituted aromatic hydrocarbon group having 6 to 18 carbon atoms are the same as described for R¹ above, which is an unsubstituted aromatic hydrocarbon group having 6 to 18 carbon atoms. Preferred examples among these include groups produced by removing b+1 hydrogen atoms from aromatic hydrocarbons such as benzene and naphthalene. A more preferred example thereof includes a phenyl group.

Specific examples of Ar¹, which is an unsubstituted heteroaromatic group having 3 to 17 carbon atoms are the same as described for R¹ above, which is an unsubstituted heteroaromatic group having 3 to 17 carbon atoms. A preferred example among these includes a group produced by removing b+1 hydrogen atoms from thiophene, benzothiophene, furan, benzofuran, or indole. A more preferred example thereof includes a group produced by removing b+1 hydrogen atoms from benzothiophene, benzofuran, or indole.

Specific examples of Ar¹, which is an unsubstituted linked aromatic group are the same as described for R¹, which is an unsubstituted linked aromatic group.

R independently represents deuterium, an aliphatic hydrocarbon group having 1 to 10 carbon atoms, a substituted or unsubstituted aromatic hydrocarbon group having 6 to 18 carbon atoms, or a substituted or unsubstituted aromatic heterocyclic group having 3 to 17 carbon atoms.

Specific examples of R, which is an aliphatic hydrocarbon group having 1 to 10 carbon atoms are the same as described for R¹ above, which is an aliphatic hydrocarbon group having 1 to 10 carbon atoms. Among these, a preferred example thereof includes a methyl group, an ethyl group, a n-propyl group, a n-butyl group, a tert-butyl group, a neopentyl group, or a cyclohexyl group.

Specific examples of R, which is an unsubstituted aromatic hydrocarbon group having 6 to 18 carbon atoms are the same as described for R¹ above, which is an unsubstituted aromatic hydrocarbon group having 6 to 18 carbon atoms. Of these, preferred examples thereof include groups produced by removing one hydrogen from aromatic hydrocarbons such as benzene and naphthalene. A more preferred example thereof includes a phenyl group.

Specific examples of R, which is an unsubstituted heteroaromatic group having 3 to 17 carbon atoms are the same as described for R¹ above, which is an unsubstituted heteroaromatic group having 3 to 17 carbon atoms. Among these, preferred examples thereof include a group produced by removing one hydrogen from thiophene, benzothiophene, dibenzothiophene, furan, benzofuran, dibenzofuran indole, or carbazole. A more preferred example thereof includes a dibenzothioenyl group, a dibenzofuranyl group, or a carbazolyl group.

Specific examples of R, which is an unsubstituted linked aromatic group are the same as described for R¹ being an unsubstituted linked aromatic group.

L¹ and L² each independently represent a direct bond, a substituted or unsubstituted aromatic hydrocarbon group having 6 to 18 carbon atoms, a substituted or unsubstituted aromatic heterocyclic group having 3 to 17 carbon atoms, or a substituted or unsubstituted linked aromatic group formed by linking 2 to 8 of these aromatic groups, and L¹ and L² preferably each independently represent a direct bond, a substituted or unsubstituted aromatic hydrocarbon group having 6 to 18 carbon atoms, or a substituted or unsubstituted aromatic heterocyclic group having 3 to 17 carbon atoms, and they more preferably represent the direct bond.

Specific examples of L¹ and L² being unsubstituted aromatic hydrocarbon groups having 6 to 18 carbon atoms are the same as described for R¹ above, which is an unsubstituted aromatic hydrocarbon group having 6 to 18 carbon atoms. Among these, preferred examples thereof include aromatic hydrocarbon groups produced by removing two hydrogen atoms from aromatic hydrocarbons selected from benzene, naphthalene, anthracene, chrysene, pyrene, phenanthrene, triphenylene, and fluorene. A more preferable example thereof includes a group produced by removing two hydrogen atoms from benzene or naphthalene.

Specific examples of L¹ and L² being unsubstituted heteroaromatic groups having 3 to 17 carbon atoms are the same as described for R¹ above, except that L¹ is divalent. Among these, preferred examples thereof include a group produced by removing two hydrogen atoms from thiophene, benzothiophene, dibenzothiophene, furan, benzofuran, dibenzofuran, or carbazole. A more preferred example thereof includes a group produced by removing two hydrogen atoms from dibenzothiophene, dibenzofuran, or carbazole.

Specific examples of L¹ and L² that are unsubstituted linked aromatic groups are the same as described for R¹ above, which is an unsubstituted linked aromatic group, except that L¹ and L² are divalent groups.

a represents the number of substitutions, and independently represents an integer of 0 to 4, more preferably a=0; b to f represent the number of substitutions, independently represent an integer of 0 to 4, and satisfy the condition that b+c+d+e+f≥1. Preferably, b+c+d≥1 and more preferably b+c+d+e+f≥2.

The compound for an organic electroluminescent device of the present invention preferably has a glass transition temperature (Tg) of 135° C. or higher and more preferably 140° C. or higher. The compound of the present invention represented by the general formula (1) above has a high glass transition temperature due to the adamantyl group, so that it has high resistance to heat generated upon operation of device, resulting in one of the reasons for the organic EL device of the present invention exhibiting long lifetime characteristics of the device.

The compound of the present invention is excellent as a host material to be used in a light emitting layer of an organic EL device. The organic EL device of the present invention is an organic electroluminescent device that includes one or more light emitting layers between an anode and a cathode opposite to each other, wherein at least one of the light emitting layers includes a host selected from the compounds represented by the general formula (1) above and a light emitting dopant. The light emitting dopant is preferably a light emitting dopant containing a boron atom.

Also, in the organic EL device of the present invention, the light emitting dopant is preferably the compound represented by the general formula (4a) or (4b) above. The compounds represented by the general formula (4a) or (4b) above will be described below.

In the general formula (4a) or (4b), the ring J, the ring K, the ring C, the ring D, the ring E, the ring F, the ring G, and the ring H are each independently a substituted or unsubstituted aromatic hydrocarbon ring having 6 to 24 carbon atoms, or a substituted or unsubstituted aromatic heterocyclic ring having 3 to 17 carbon atoms, preferably an aromatic hydrocarbon ring having 6 to 20 carbon atoms, or an aromatic heterocyclic ring having 3 to 15 carbon atoms, and more preferably an aromatic hydrocarbon ring having 6 to 20 carbon atoms. As described above, the C to K rings represent the aromatic hydrocarbon ring or aromatic heterocyclic ring, and are therefore collectively referred to as aromatic rings in the present description.

Specific examples of the aromatic ring include a ring including benzene, naphthalene, acenaphthene, acenaphthylene, azulene, anthracene, chrysene, pyrene, phenanthrene, triphenylene, fluorene, benzo[a]anthracenepyridine, pyridine, pyrimidine, triazine, thiophene, isothiazole, thiazole, pyridazine, pyrrole, pyrazole, imidazole, triazole, thiadiazole, pyrazine, furan, isoxazole, quinoline, isoquinoline, quinoxaline, quinazoline, thiadiazole, phthalazine, tetrazole, indole, benzofuran, benzothiophene, benzoxazole, benzothiazole, indazole, benzimidazole, benzotriazole, benzisothiazole, benzothiadiazole, purine, pyranone, coumarin, isocoumarin, chromone, dibenzofuran, dibenzothiophene, dibenzoselenophene, or carbazole. The aromatic ring is more preferably a benzene ring, a naphthalene ring, an anthracene ring, a triphenylene ring, a phenanthrene ring, a pyrene ring, a pyridine ring, a dibenzofuran ring, a dibenzothiophene ring, or a carbazole ring.

Y¹ each independently is B, P, P═O, P═S, Al, Ga, As, Si—R³ or Ge—R³, preferably B, P, P═O or P═S, and more preferably B.

R³ each independently is an aliphatic hydrocarbon group having 1 to 10 carbon atoms, a substituted or unsubstituted aromatic hydrocarbon group having 6 to 18 carbon atoms, or a substituted or unsubstituted aromatic heterocyclic group having 3 to 17 carbon atoms, and preferably an aliphatic hydrocarbon group having 1 to 8 carbon atoms, a substituted or unsubstituted aromatic hydrocarbon group having 6 to 12 carbon atoms, or a substituted or unsubstituted aromatic heterocyclic group having 3 to 15 carbon atoms. R³ is more preferably a substituted or unsubstituted aromatic hydrocarbon group having 6 to 10 carbon atoms, or a substituted or unsubstituted aromatic heterocyclic group having 3 to 12 carbon atoms.

Specific examples of R³, which is an aliphatic hydrocarbon group having 1 to 10 carbon atoms are the same as described for R¹ in the general formula (1) above, which is an aliphatic hydrocarbon group having 1 to 10 carbon atoms. Of these, a preferred example thereof includes a methyl group, an ethyl group, a n-propyl group, a n-butyl group, a tert-butyl group, a neopentyl group, or a cyclohexyl group.

Specific examples of R³, which is an unsubstituted aromatic hydrocarbon group having 6 to 18 carbon atoms are the same as described for R¹ in the general formula (1) above, which is an aromatic hydrocarbon group having 6 to 18 carbon atoms. Of these, preferred examples thereof include a group produced by removing one hydrogen atom from benzene, naphthalene, anthracene, chrysene, pyrene, phenanthrene, triphenylene, or fluorene. A more preferred example thereof includes a phenyl group or a naphthyl group.

Specific examples of R³, which is an unsubstituted heteroaromatic group having 3 to 17 carbon atoms are the same as described for R¹ in the general formula (1) above, which is an unsubstituted heteroaromatic group having 3 to 17 carbon atoms. Among these, preferred examples thereof include a group produced by removing one hydrogen from thiophene, benzothiophene, dibenzothiophene, furan, benzofuran, dibenzofuran, or carbazole. More preferred examples thereof include a dibenzothienyl group, a dibenzofuranyl group, or a carbazolyl group.

X² is each independently O, N—Ar⁴, S or Se, preferably O, N—Ar⁴ or S, and more preferably O or N—Ar⁴.

N—Ar⁴ may be bonded to any of the ring J, the ring K, the ring C, the ring D, the ring E, the ring F, the ring G, or the ring H to form a heterocyclic ring including N.

Ar⁴ is each independently a substituted or unsubstituted aromatic hydrocarbon group having 6 to 18 carbon atoms, a substituted or unsubstituted aromatic heterocyclic group having 3 to 17 carbon atoms, or a substituted or unsubstituted linked aromatic group formed by linking 2 to 8 of these groups, and preferably a substituted or unsubstituted aromatic hydrocarbon group having 6 to 12 carbon atoms, a substituted or unsubstituted aromatic heterocyclic group having 3 to 15 carbon atoms, or a substituted or unsubstituted linked aromatic group formed by linking 2 to 6 aromatic rings thereof. More preferably Ar⁴ is a substituted or unsubstituted aromatic hydrocarbon group having 6 to 10 carbon atoms, a substituted or unsubstituted aromatic heterocyclic group having 3 to 12 carbon atoms, or a substituted or unsubstituted linked aromatic group formed by linking 2 to 4 aromatic rings thereof. Still more preferably, Ar⁴ is a substituted or unsubstituted aromatic hydrocarbon group having 6 to 10 carbon atoms.

Specific examples of Ar⁴, which is an unsubstituted aromatic hydrocarbon group having 6 to 18 carbon atoms are the same as described for R¹ in general formula (1) above, which is an unsubstituted aromatic hydrocarbon group having 6 to 18 carbon atoms. Of these, preferred examples thereof include a group produced by removing one hydrogen from benzene, naphthalene, anthracene, chrysene, pyrene, phenanthrene, triphenylene, or fluorene. More preferred examples thereof include a phenyl group or a naphtyl group.

Specific examples of Ar⁴, which is an unsubstituted heteroaromatic group having 3 to 17 carbon atoms are the same as described for R¹ in the general formula (1) above, which is an unsubstituted heteroaromatic group having 3 to 17 carbon atoms. Among these, preferred examples thereof include a group produced by removing one hydrogen from thiophene, benzothiophene, dibenzothiophene, furan, benzofuran, dibenzofuran, or carbazole. More preferred examples thereof include a dibenzothienyl group, a dibenzofuranyl group, or a carbazolyl group.

Specific examples of Ar⁴, which is an unsubstituted linked aromatic group are the same as described for R¹ in the general formula (1) above, which is an unsubstituted linked aromatic group.

R⁴ each independently represents a cyano group, deuterium, a diarylamino group having 12 to 44 carbon atoms, an arylheteroarylamino group having 12 to 44 carbon atoms, an aliphatic hydrocarbon group having 1 to 10 carbon atoms, a substituted or unsubstituted aromatic hydrocarbon group having 6 to 18 carbon atoms, a substituted or unsubstituted aromatic heterocyclic group having 3 to 17 carbon atoms. R⁴ is preferably a diarylamino group having 12 to 36 carbon atoms, an arylheteroarylamino group having 12 to 36 carbon atoms, a diheteroarylamino group having 12 to 36 carbon atoms, a substituted or unsubstituted aromatic hydrocarbon group having 6 to 12 carbon atoms, or a substituted or unsubstituted aromatic heterocyclic ring having 3 to 15 carbon atoms. R⁴ is more preferably a diarylamino group having 12 to 24 carbon atoms, an arylheteroarylamino group having 12 to 24 carbon atoms, a diheteroarylamino group having 12 to 24 carbon atoms, a substituted or unsubstituted aromatic hydrocarbon group having 6 to 10 carbon atoms, or a substituted or unsubstituted aromatic heterocyclic group having 3 to 12 carbon atoms.

Specific examples of R⁴ in case of representing a diarylamino group having 12 to 44 carbon atoms, an arylheteroarylamino group having 12 to 44 carbon atoms, a diheteroarylamino group having 12 to 44 carbon atoms, or an aliphatic hydrocarbon group having 1 to 10 carbon atoms, include a diphenylamino group, a dibiphenylamino group, a phenylbiphenylamino group, a naphthylphenylamino group, a dinaphthylamino group, a dianthranylamino group, a diphenanthrenylamino group, a dipyrenylamino group, a dibenzofuranylphenylamino group, a dibenzofuranylbiphenylamino group, a dibenzofuranylnaphthylamino group, a dibenzofuranylanthranylamino group, a dibenzofuranylphenanthrenylamino group, a dibenzofuranylpyrenylamino group, a bisdibenzofuranylamino group, a carbazolylphenylamino group, a carbazolylnaphthylamino group, a carbazolylanthranylamino group, a carbazolylphenanthrenylamino group, a carbazolylpyrenylamino group, a dicarbazolylamino group, a methyl group, an ethyl group, a propyl group, a butyl group, a pentyl group, a hexyl group, a heptyl group, an octyl group, and a nonyl group. Preferred examples thereof include a diphenylamino group, a dibiphenylamino group, a phenylbiphenylamino group, a naphthylphenylamino group, a dinaphthylamino group, a dianthranylamino group, a diphenanthrenylamino group, and a dipyrenylamino group, and a more preferred example thereof includes a diphenylamino group, a dibiphenylamino group, a phenylbiphenylamino group, a naphthylphenylamino group, a dinaphthylamino group, a dibenzofuranylphenylamino group, or a carbazolylphenylamino group.

Specific examples of R⁴, which is an unsubstituted aromatic hydrocarbon group having 6 to 18 carbon atoms are the same as described for R¹ in general formula (1) above, which is an unsubstituted aromatic hydrocarbon group having 6 to 18 carbon atoms. Of these, preferred examples thereof include a group produced by removing one hydrogen from benzene, naphthalene, anthracene, chrysene, pyrene, phenanthrene, triphenylene, or fluorene. More preferred examples thereof include a phenyl group or a naphtyl group.

Specific examples of R⁴, which is an unsubstituted heteroaromatic group having 3 to 17 carbon atoms are the same as described for R¹ in the general formula (1) above, which is an unsubstituted heteroaromatic group having 3 to 17 carbon atoms. Of these, preferred examples thereof include a group produced by removing one hydrogen from thiophene, benzothiophene, dibenzothiophene, furan, benzofuran, dibenzofuran, or carbazole. More preferred examples thereof include a dibenzothienyl group, a dibenzofuranyl group, or a carbazolyl group.

g and h represent the number of substitutions, and each independently represent an integer of 0 to 4, preferably an integer of 0 to 2, and more preferably an integer of 0 or 1. i and j represent the number of substitutions, and each independently represent an integer of 0 to 3, preferably an integer of 0 to 2, and more preferably an integer of 0 or 1. k represents the number of substitutions, and an integer of 0 to 2 and preferably an integer of 0 or 1.

A preferred example of the polycyclic aromatic compound represented by the general formula (4a) above includes a boron-containing polycyclic aromatic compound represented by the following formula (5a), and a preferred example of the polycyclic aromatic compound represented by the general formula (4b) above includes a boron-containing polycyclic aromatic compound represented by the following formula (5b).

In the compound represented by the general formulae (5a) and (5b) above, X³ each independently represents N—Ar⁴, O, or S, and at least one X³ represents N—Ar⁴. The symbol common to the general formula (4a) or general formula (4b) has the same meaning.

The organic electroluminescent device of the present invention is an organic electroluminescent device that includes one or more light emitting layers between an anode and a cathode opposite to each other, wherein at least one of the light emitting layers includes a first host selected from the compounds represented by the general formula (1) above, a second host, and a light emitting dopant containing a boron atom. The second host is preferably the compound represented by the general formula (6) above.

In the general formula (6), Z is an indolocarbazole ring-containing group represented by formula (7), and “* *” in the formula represents a bonding site to L³. The ring A in the formula is a heterocyclic ring represented by the general formula (8) and the ring A is condensed with the adjacent ring at arbitrary position thereof. Z is preferably an indolocarbazole ring-containing group represented by the following general formula (101).

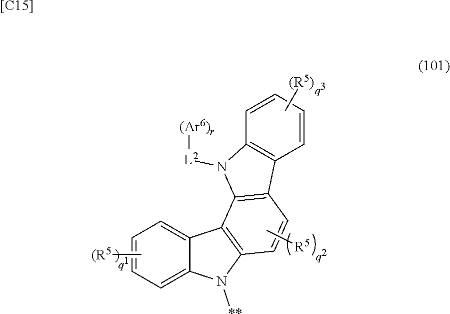

wherein “**” in the formula (101) represents a bonding site to L³.

L³ and L⁴ are each independently a substituted or unsubstituted aromatic hydrocarbon group having 6 to 18 carbon atoms or a substituted or unsubstituted aromatic heterocyclic group having 3 to 17 carbon atoms, preferably a substituted or unsubstituted aromatic hydrocarbon group having 6 to 12 carbon atoms or a substituted or unsubstituted aromatic heterocyclic group having 3 to 15 carbon atoms. More preferably L¹ and L² are each a substituted or unsubstituted aromatic hydrocarbon group having 6 to 10 carbon atoms or a substituted or unsubstituted aromatic heterocyclic group having 3 to 12 carbon atoms.

Specific examples of L³ and L⁴ that are unsubstituted aromatic hydrocarbon groups having 6 to 18 carbon atoms are the same as described for R¹ in general formula (1) above, which is an unsubstituted aromatic hydrocarbon group having 6 to 18 carbon atoms. Of these, preferred examples thereof include benzene, naphthalene, anthracene, chrysene, pyrene, phenanthrene, triphenylene, or fluorene. More preferred examples thereof include benzene or naphthalene. It is to be noted that L³ is a v+w valent group, and L⁴ is an r+1 valent group.

Specific examples of L³ and L⁴ that are unsubstituted heteroaromatic groups having 3 to 17 carbon atoms are the same as described for R¹ in general formula (1) above, which is an unsubstituted heteroaromatic group having 3 to 17 carbon atoms. Among these, preferred examples thereof include groups produced by removing v+w hydrogen atoms for L³ and r+1 hydrogen atoms for L⁴ from thiophene, benzothiophene, dibenzothiophene, furan, benzofuran, dibenzofuran, or carbazole. More preferred examples thereof include a dibenzothienyl group, a dibenzofuranyl group, or a carbazolyl group.

Ar⁵ and Ar⁶ are each independently deuterium, a substituted or unsubstituted aromatic hydrocarbon group having 6 to 18 carbon atoms, a substituted or unsubstituted aromatic heterocyclic group having 3 to 17 carbon atoms, or a substituted or unsubstituted linked aromatic group formed by linking 2 to 8 of these aromatic groups, preferably a substituted or unsubstituted aromatic hydrocarbon group having 6 to 12 carbon atoms, a substituted or unsubstituted aromatic heterocyclic group having 3 to 15 carbon atoms, or a substituted or unsubstituted linked aromatic group formed by linking 2 to 4 of these groups, and more preferably a substituted or unsubstituted aromatic hydrocarbon group having 6 to 10 carbon atoms, a substituted or unsubstituted aromatic heterocyclic group having 3 to 12 carbon atoms, or a substituted or unsubstituted linked aromatic group formed by linking 2 to 3 of these groups. Still more preferred examples thereof include a substituted or unsubstituted aromatic hydrocarbon group having 6 to 10 carbon atoms or a substituted or unsubstituted aromatic heterocyclic group having 3 to 12 carbon atoms.

Specific examples of Ar⁵ and Ar⁶ that are unsubstituted aromatic hydrocarbon groups having 6 to 18 carbon atoms are the same as described for R¹ in the general formula (1) above, which is an unsubstituted aromatic hydrocarbon group having 6 to 18 carbon atoms. Of these, preferred examples thereof include a group produced by removing one hydrogen from benzene, naphthalene, anthracene, chrysene, pyrene, phenanthrene, triphenylene, or fluorene. More preferred examples thereof include a phenyl group or a naphtyl group.

Specific examples of Ar⁵ and Ar⁶ that are unsubstituted heteroaromatic groups having 3 to 17 carbon atoms are the same as described for R¹ in the general formula (1) above, which is an unsubstituted heteroaromatic group having 3 to 17 carbon atoms. Of these, preferred examples thereof include a group produced by removing one hydrogen from thiophene, benzothiophene, dibenzothiophene, furan, benzofuran, dibenzofuran, or carbazole. More preferred examples thereof include a dibenzothienyl group, a dibenzofuranyl group, or a carbazolyl group.

Specific examples of Ar⁵ and Ar⁶ that are unsubstituted linked aromatic groups are the same as described for R¹ in the general formula (1) above, which is an unsubstituted linked aromatic group.

R⁵ independently is deuterium, an aliphatic hydrocarbon group having 1 to 10 carbon atoms, a substituted or unsubstituted aromatic hydrocarbon group having 6 to 18 carbon atoms, or a substituted or unsubstituted aromatic heterocyclic group having 3 to 17 carbon atoms, and is preferably deuterium, a substituted or unsubstituted aromatic hydrocarbon group having 6 to 12 carbon atoms, or a substituted or unsubstituted aromatic heterocyclic ring having 3 to 15 carbon atoms. More preferably, R⁵ is deuterium, a substituted or unsubstituted aromatic hydrocarbon group having 6 to 10 carbon atoms or a substituted or unsubstituted aromatic heterocyclic group having 3 to 12 carbon atoms.

Specific examples of R⁵, which is an aliphatic hydrocarbon group having 1 to 10 carbon atoms are the same as described for R¹ in the general formula (1) above, which is an aliphatic hydrocarbon group having 1 to 10 carbon atoms. Of these, a preferred example thereof includes a methyl group, an ethyl group, a n-propyl group, a n-butyl group, a tert-butyl group, a neopentyl group, or a cyclohexyl group.

Specific examples of R⁵, which is an unsubstituted aromatic hydrocarbon group having 6 to 18 carbon atoms are the same as described for R¹ in the general formula (1) above, which is an unsubstituted aromatic hydrocarbon group having 6 to 18 carbon atoms. Of these, preferred examples thereof include a group produced by removing one hydrogen atom from benzene, naphthalene, anthracene, chrysene, pyrene, phenanthrene, triphenylene, or fluorene. A more preferred example thereof includes a phenyl group or a naphthyl group.

Specific examples of R⁵, which is an unsubstituted heteroaromatic group having 3 to 17 carbon atoms are the same as described for R¹ in the general formula (1) above, which is an unsubstituted heteroaromatic group having 3 to 17 carbon atoms. Among them, preferred examples thereof include a group produced by removing one hydrogen from thiophene, benzothiophene, dibenzothiophene, furan, benzofuran, dibenzofuran, or carbazole. More preferred examples thereof include a dibenzothienyl group, a dibenzofuranyl group, or a carbazolyl group.

v represents the number of substitutions, represents an integer of 1 to 3, and is preferably represented by 1 or 2. w represents the number of substitutions, represents an integer of 0 to 3, and is preferably 0 to 2. q¹ and q³ represent the number of substitutions and each independently represent an integer of 0 to 4, and is preferably represented by an integer of 0 to 2. q² represents the number of substitutions and represents an integer of 0 to 2, and is preferably represented by 0 or 1. r represents the number of substitutions and represents an integer of 0 to 3, and is preferably represented by 0 to 2.

A preferred aspect of the general formula (6) above is the general formula (6a) or (6b) above. In the general formulas (6a) and (6b) above, X⁴ represents O or S. The symbols common to the general formula (6) have the same meaning.

In the general formula (6b), R⁶ is each independently deuterium, an aliphatic hydrocarbon group having 1 to 10 carbon atoms, a substituted or unsubstituted aromatic hydrocarbon group having 6 to 18 carbon atoms, or a substituted or unsubstituted aromatic heterocyclic group having 3 to 17 carbon atoms, preferably deuterium, a substituted or unsubstituted aromatic hydrocarbon group having 6 to 12 carbon atoms, or a substituted or unsubstituted aromatic heterocyclic ring having 3 to 15 carbon atoms. The R⁶ is more preferably deuterium, a substituted or unsubstituted aromatic hydrocarbon group having 6 to 10 carbon atoms, or a substituted or unsubstituted aromatic heterocyclic group having 3 to 12 carbon atoms.

Specific examples of R⁶, which is an aliphatic hydrocarbon group having 1 to 10 carbon atoms are the same as described for R¹ in the general formula (1) above, which is an aliphatic hydrocarbon group having 1 to 10 carbon atoms. Of these, a preferred example thereof includes a methyl group, an ethyl group, a n-propyl group, a n-butyl group, a tert-butyl group, a neopentyl group, and a cyclohexyl group.

Specific examples of R⁶, which is an unsubstituted aromatic hydrocarbon group having 6 to 18 carbon atoms are the same as described for R¹ in the general formula (1) above, which is an unsubstituted aromatic hydrocarbon group having 6 to 18 carbon atoms. Of these, preferred examples thereof include a group produced by removing one hydrogen atom from benzene, naphthalene, anthracene, chrysene, pyrene, phenanthrene, triphenylene, or fluorene. A more preferred example thereof includes a phenyl group or a naphthyl group.

Specific examples of R⁶, which is an unsubstituted heteroaromatic group having 3 to 17 carbon atoms are the same as described for R¹ in the general formula (1) above, which is an unsubstituted heteroaromatic group having 3 to 17 carbon atoms. Among these, preferred examples thereof include a group produced by removing one hydrogen atom from thiophene, benzothiophene, dibenzothiophene, furan, benzofuran, dibenzofuran, or carbazole. More preferred examples thereof include a dibenzothienyl group, a dibenzofuranyl group, or a carbazolyl group.

The linked aromatic group as used herein refers to a group in which aromatic rings of an aromatic hydrocarbon group or an aromatic heterocyclic group are linked by a single bond, which may be linked in a linear or branched manner. The linked aromatic rings may be the same or different. When a group falls within the linked aromatic group, the group is considered not to be a substituted aromatic hydrocarbon group or aromatic heterocyclic group.

In the general formulae (1), (2), (3), (4a), (4b), (5a), (5b), (6), (6a), (6b), (7), (8) and (101), when Ar¹ to Ar⁵, R¹ to R⁶, L¹ to L⁴ are aromatic hydrocarbon groups, aromatic heterocyclic groups, or linked aromatic groups, they may have a substituent, and the substituent is preferably deuterium, a triarylsilyl group having 18 to 36 carbon atoms, an aliphatic hydrocarbon group having 1 to 10 carbon atoms, a diarylamino group having 12 to 44 carbon atoms. Here, the substituent is the aliphatic hydrocarbon group having 1 to 10 carbon atoms, the substituent may be linear, branched, or cyclic. The number of substituents is 0 to 5, and preferably 0 to 2. When each of the aromatic hydrocarbon groups and aromatic heterocyclic groups has a substituent, the number of carbon atoms of the substituent is not included in the calculation of the number of carbon atoms. However, it is preferred that the total number of carbon atoms including the number of carbon atoms of the substituent satisfy the above range.

Specific examples of the above-described substituents include deuterium, a methyl group, an ethyl group, a propyl group, an i-propyl group, a butyl group, a t-butyl group, a pentyl group, a cyclopentyl group, a hexyl group, a cyclohexyl group, a heptyl group, an octyl group, a nonyl group, a decyl group, a diphenylamino group, a naphthylphenylamino group, a dinaphthylamino group, a dianthranylamino group, a diphenanthrenylamino group, a dipyrenylamino group, and a triphenylsilyl group. A preferred example thereof includes deuterium, a methyl group, an ethyl group, a propyl group, a butyl group, a pentyl group, a hexyl group, a heptyl group, an octyl group, a diphenylamino group, a naphthylphenylamino group, a dinaphthylamino group, or a triphenylsilyl group.

In the present description, hydrogen in the general formulae (1), (2), (3), (4a), (4b), (5a), (5b), (6), (6a), (6b), (7), and (8), and formula (101), may be partially or all deuterium.

Specific examples of the compounds represented by the general formula (1) above are shown below, but are not limited to these exemplified compounds.

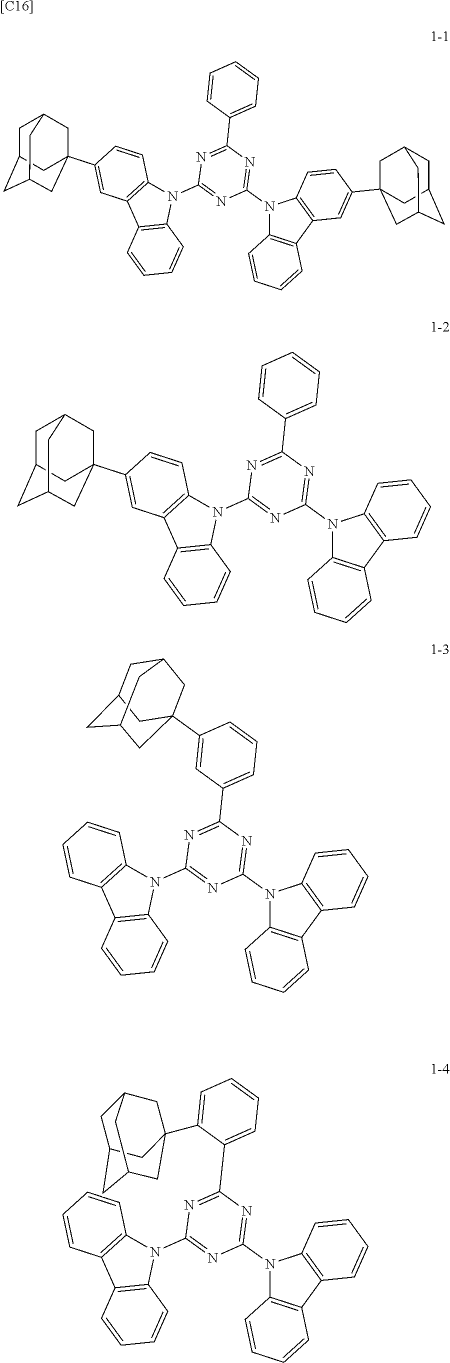

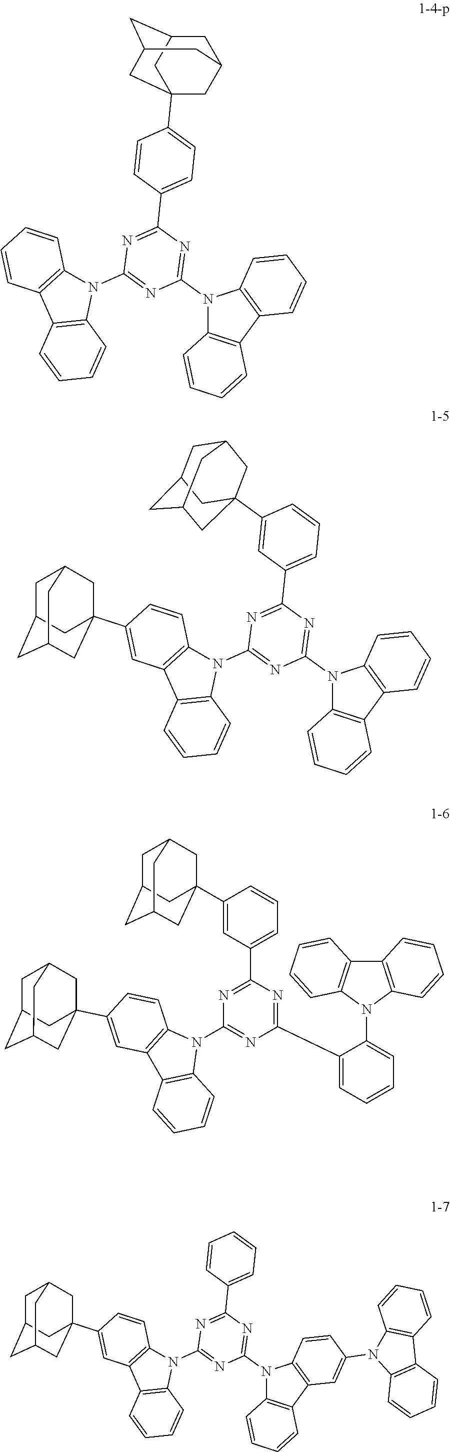

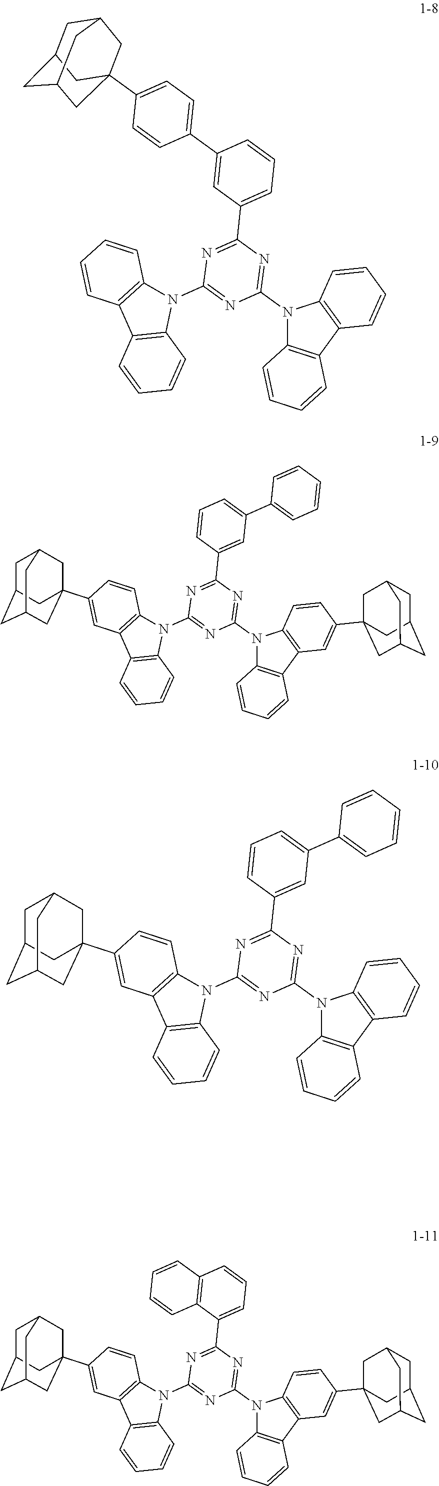

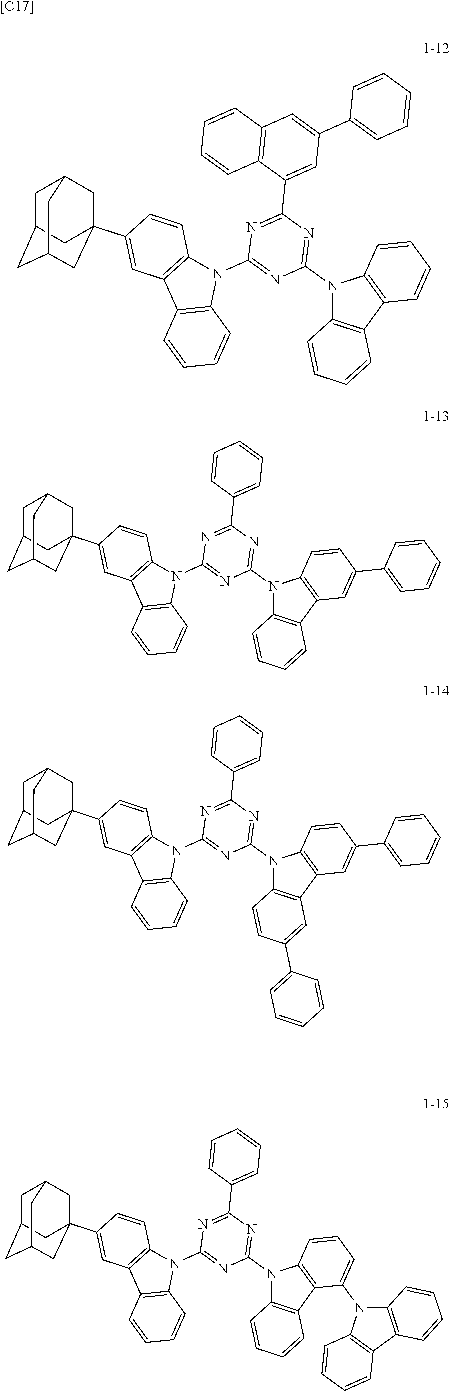

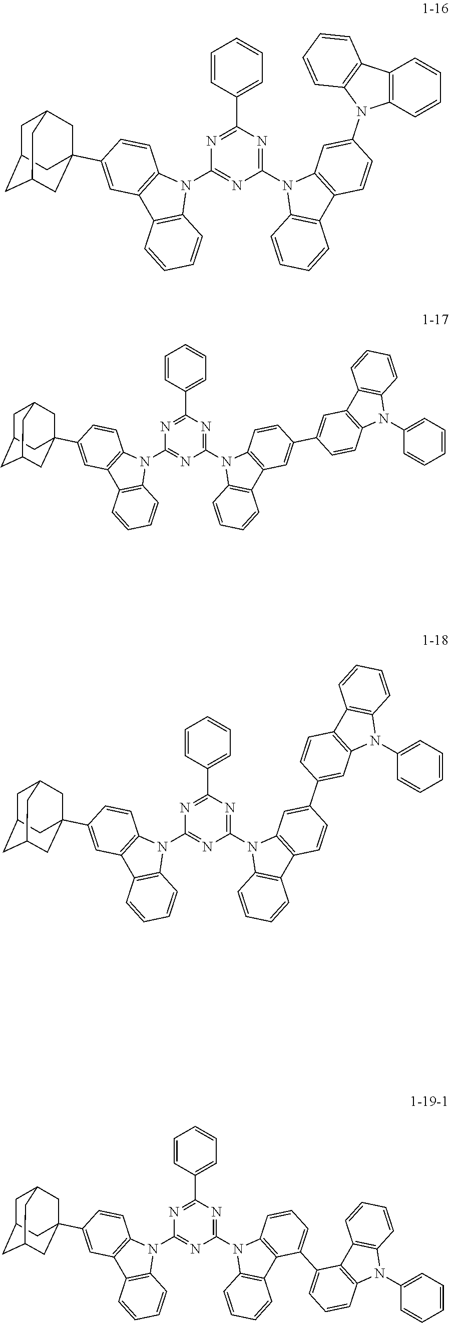

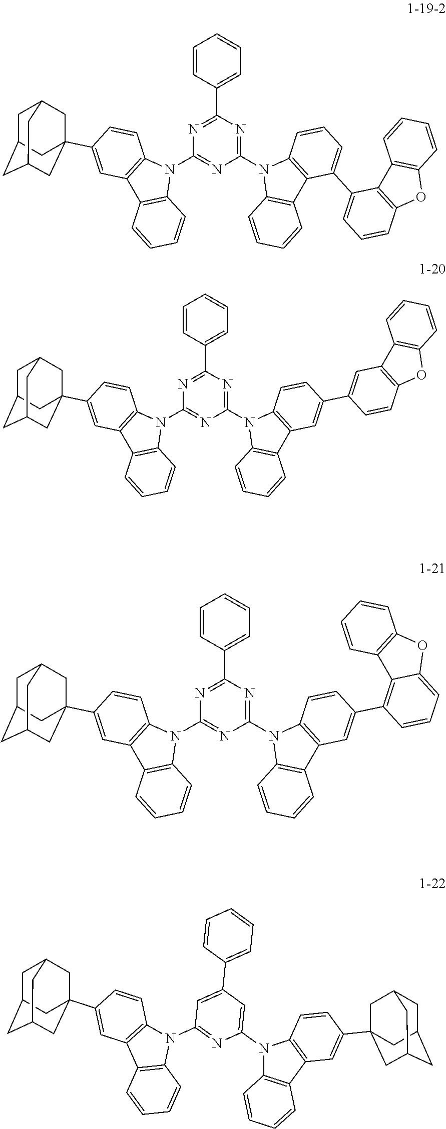

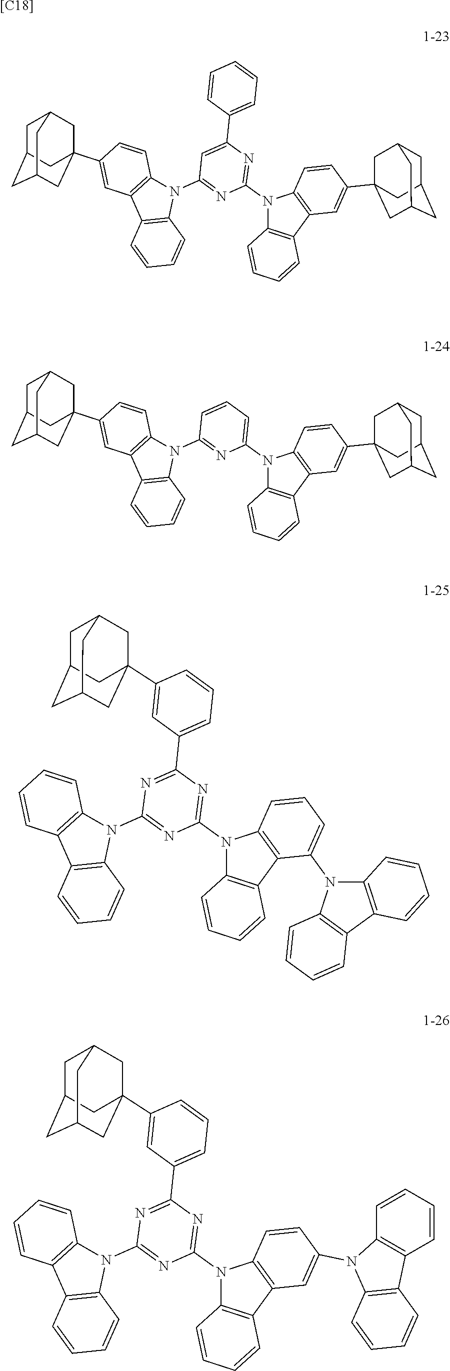

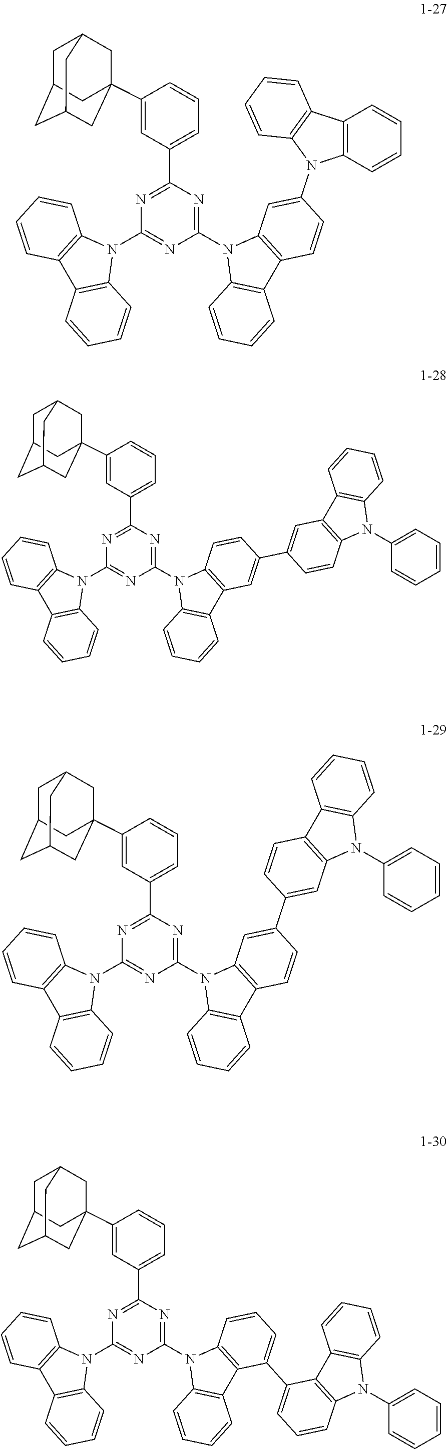

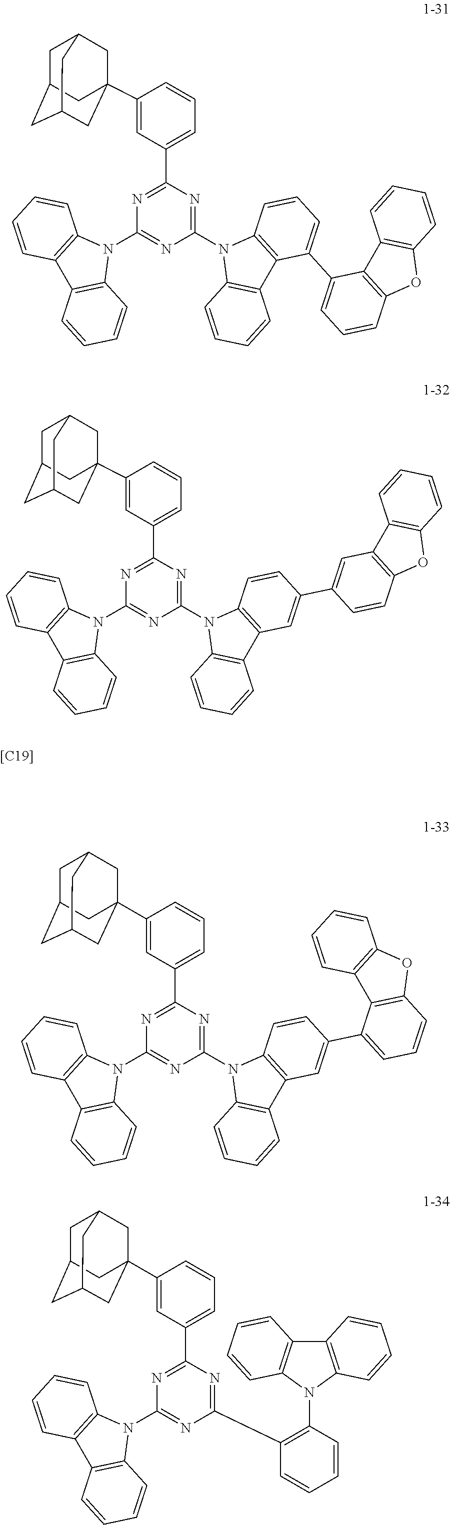

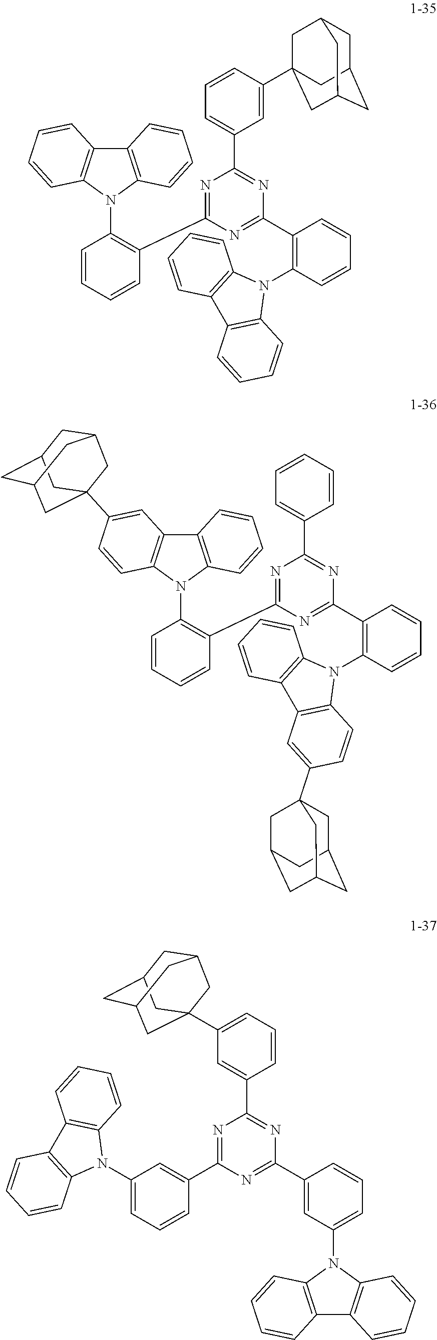

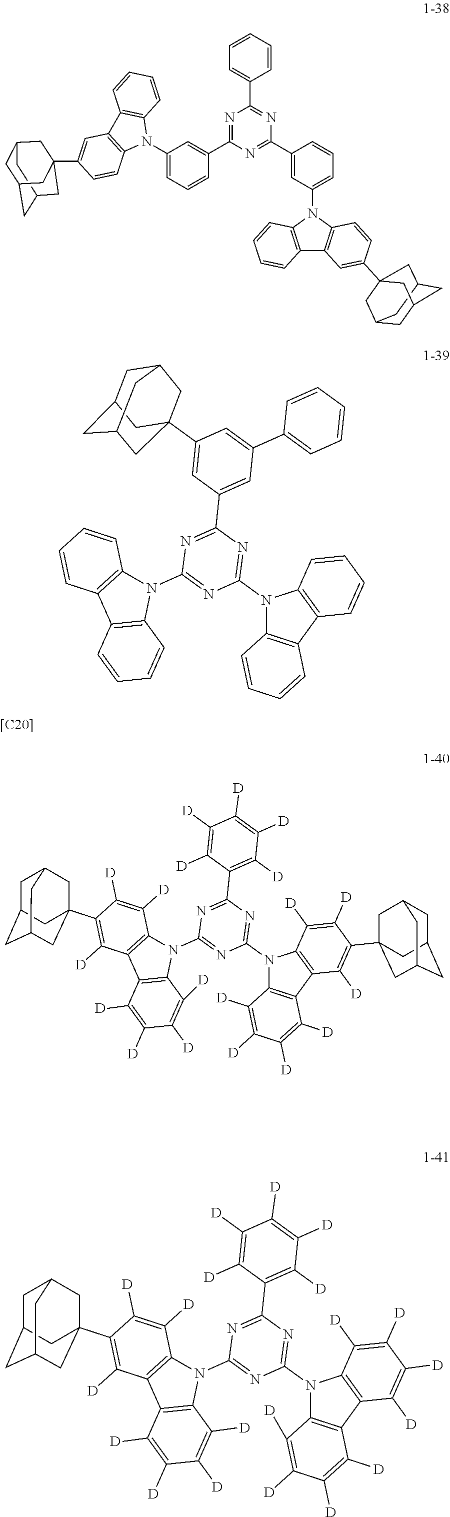

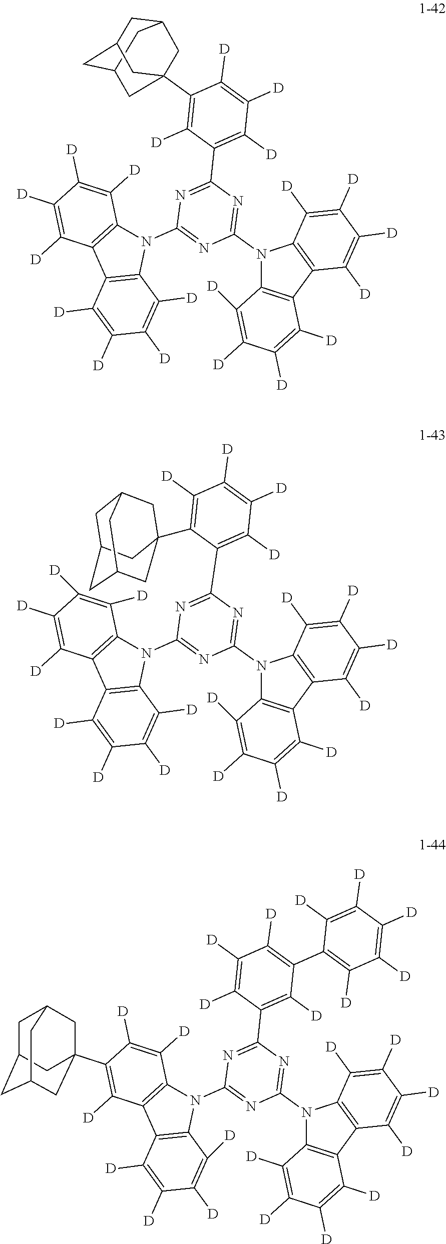

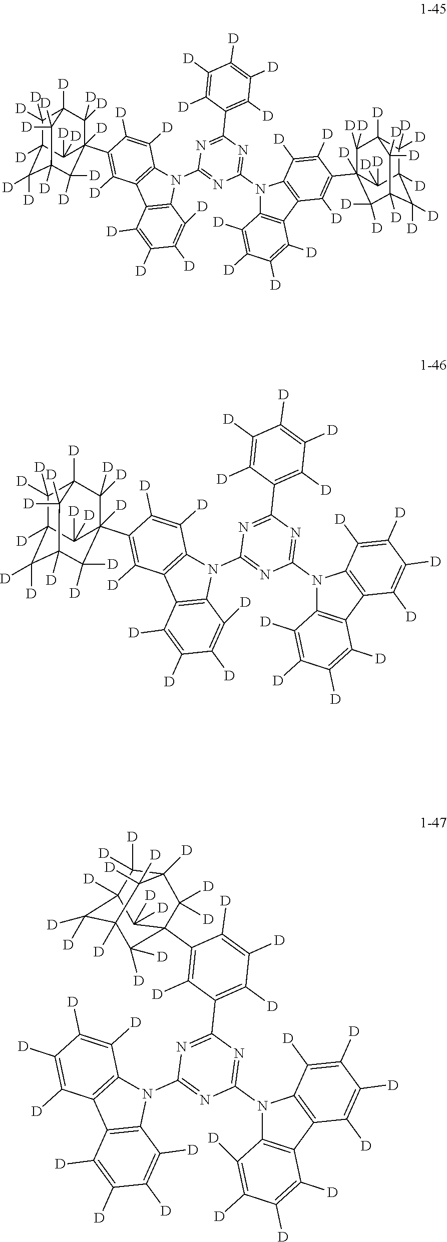

Specific examples of the compounds represented by the general formula (4a) or (4b) above, and (5a) or (5b), which is a preferred aspect of (4a) or (4b), respectively, will be described below, but are not limited to these exemplary compounds.

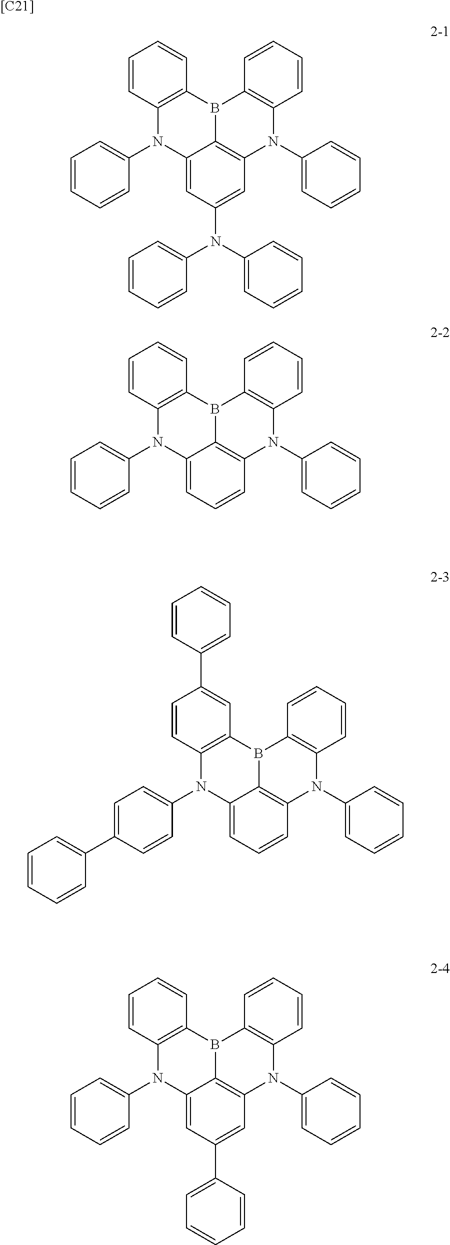

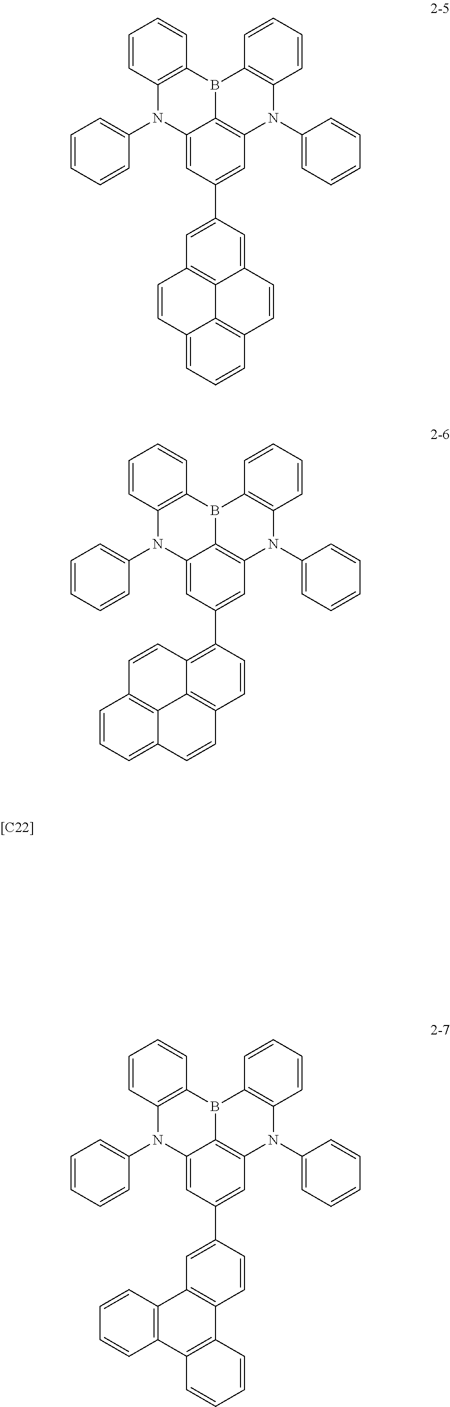

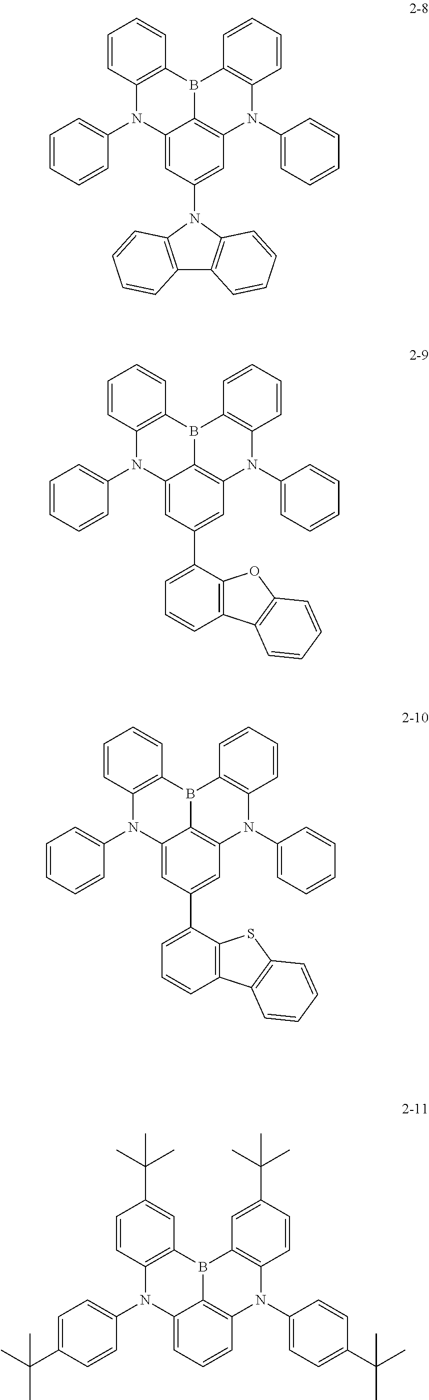

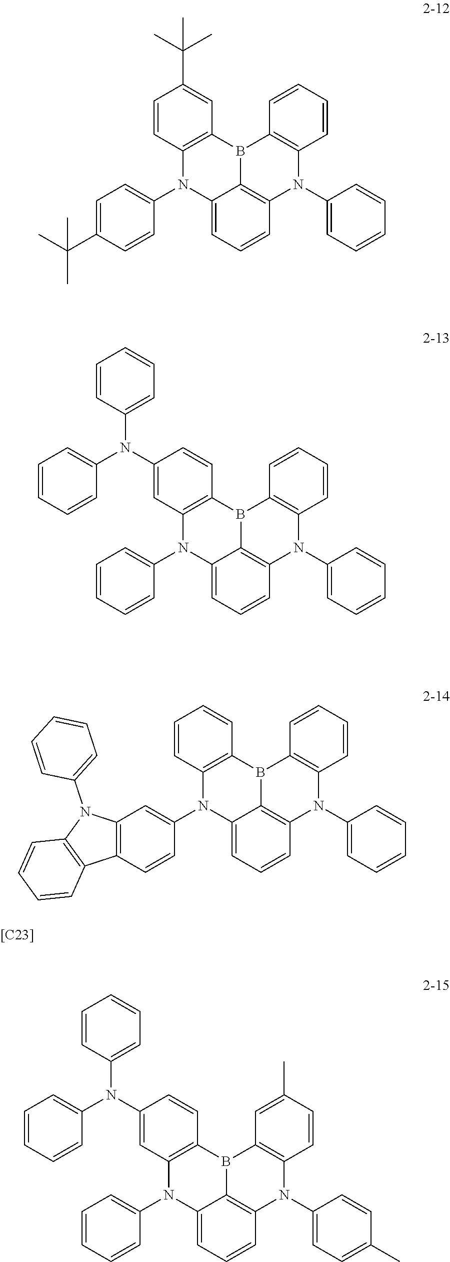

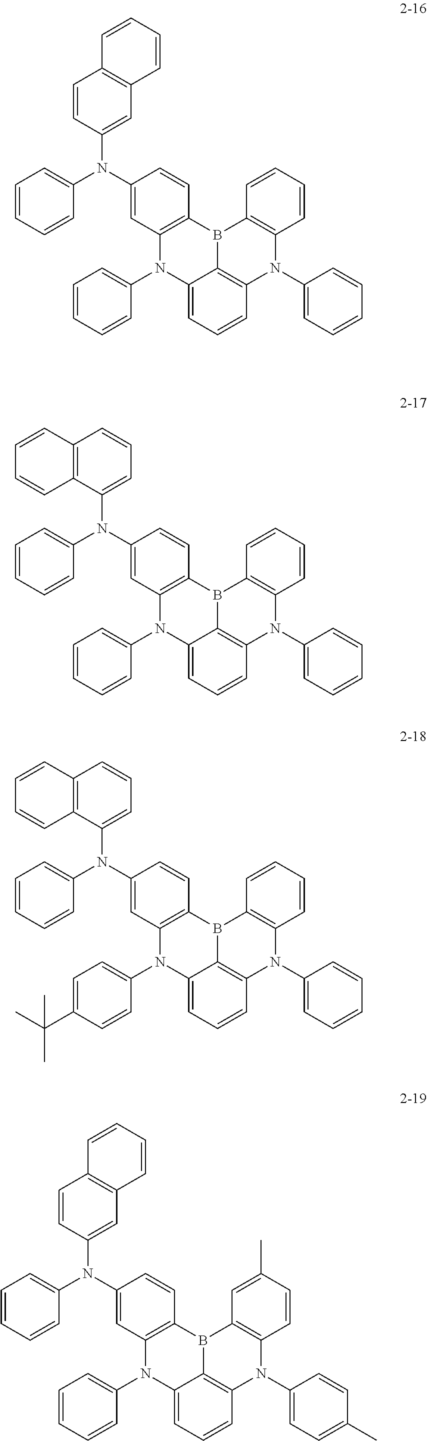

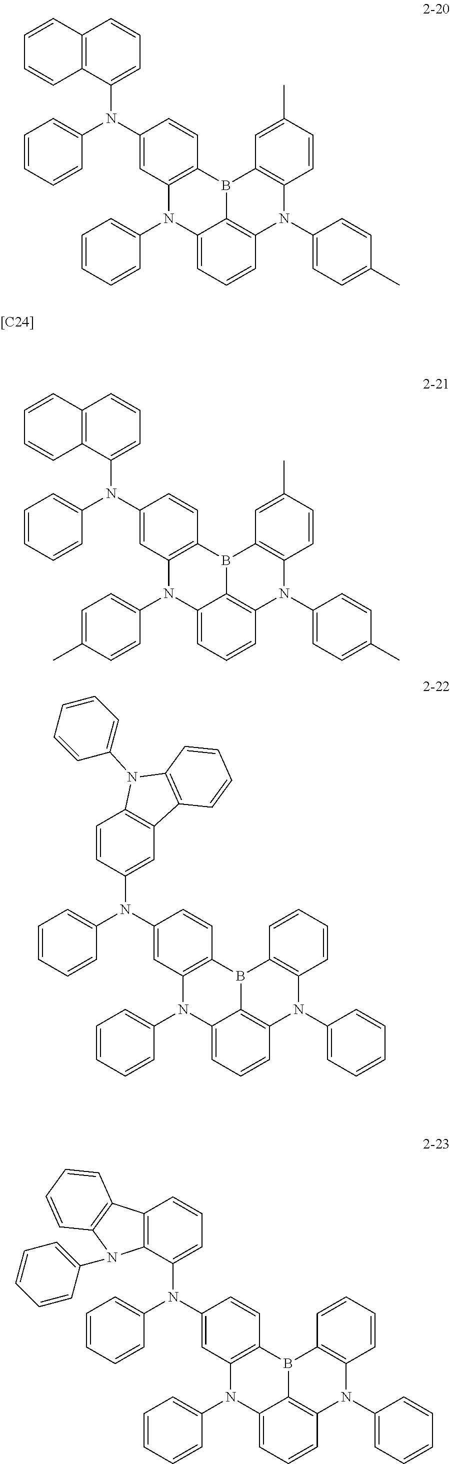

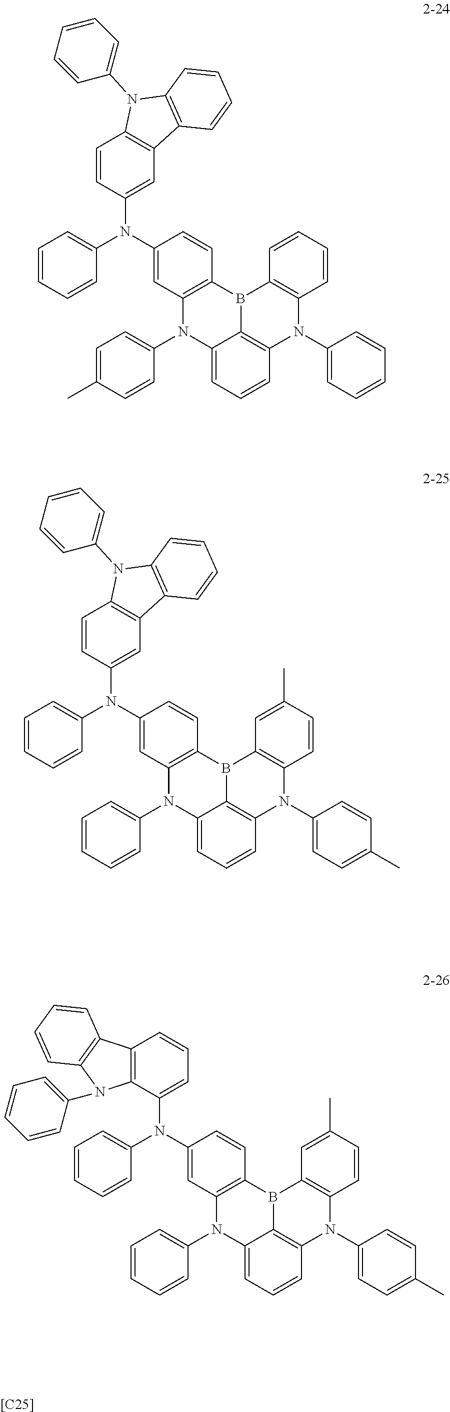

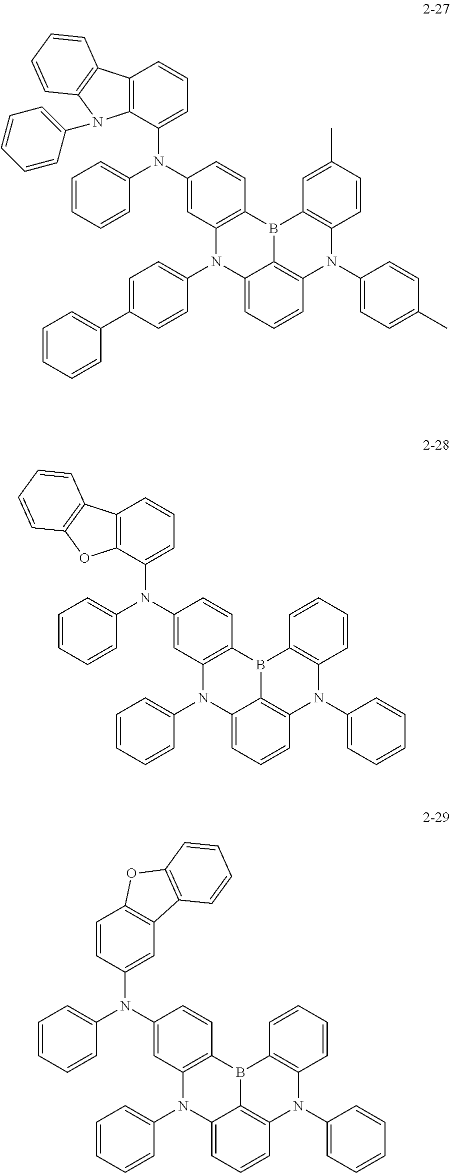

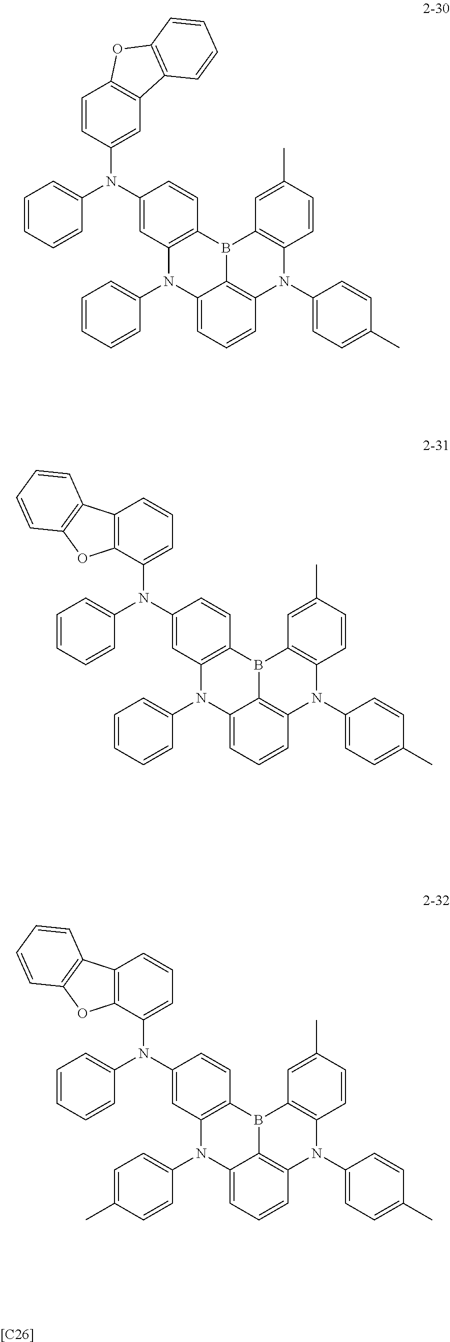

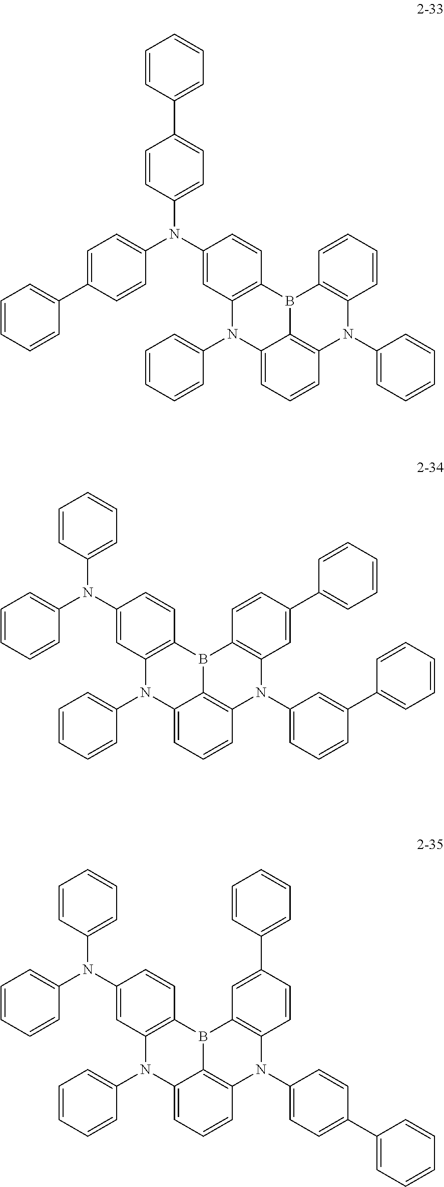

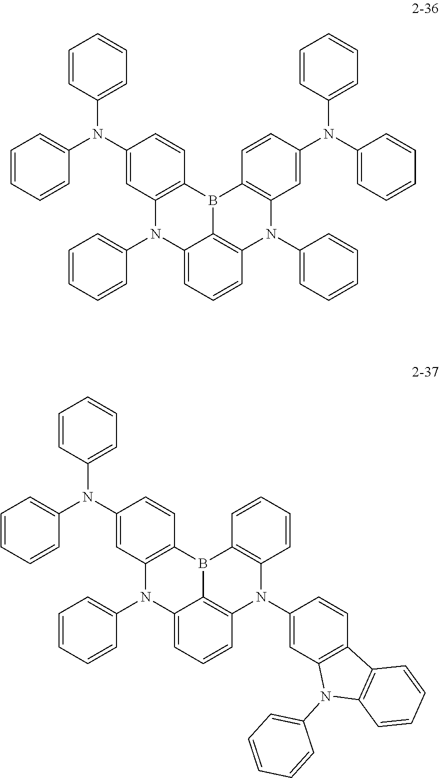

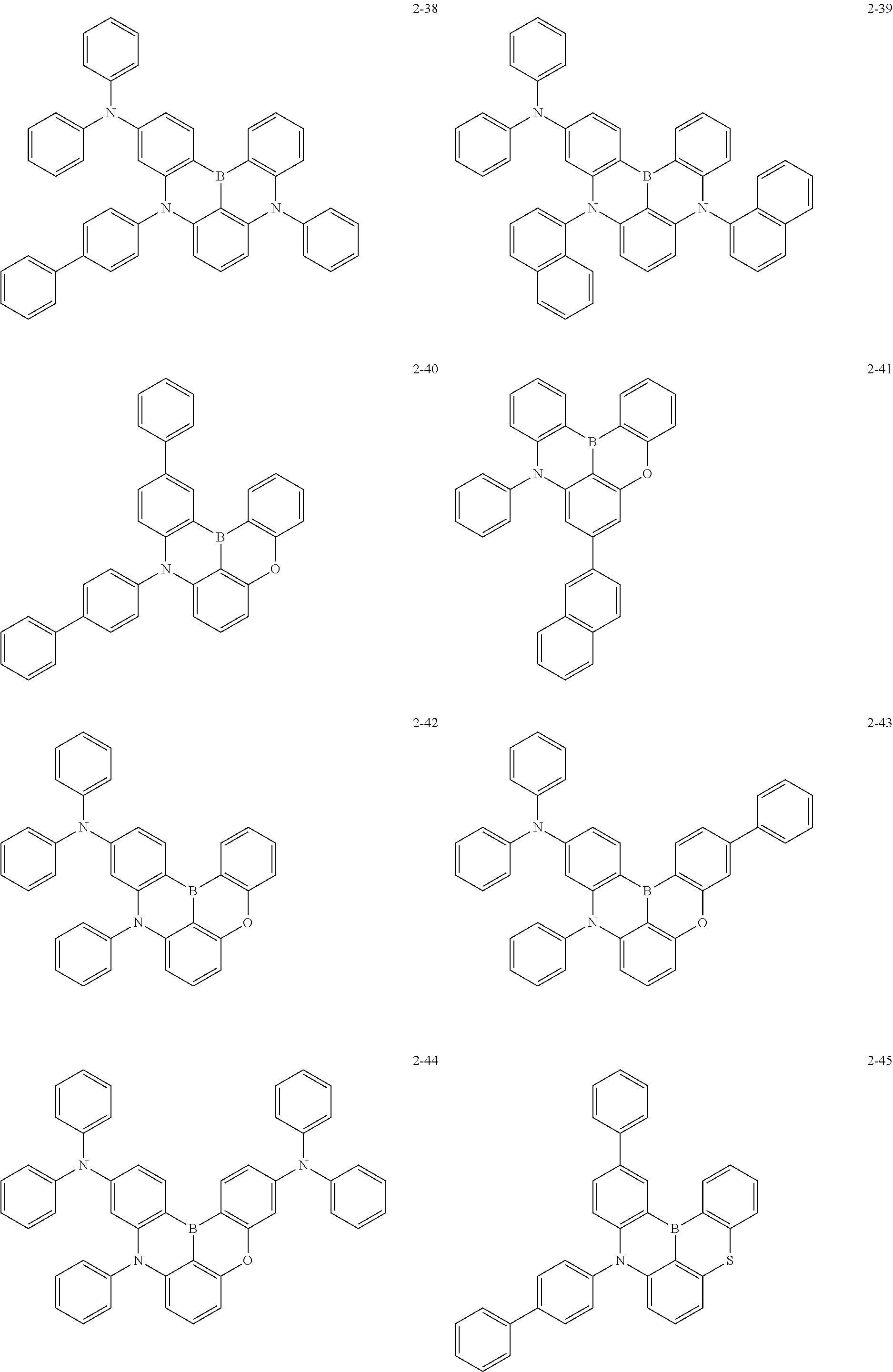

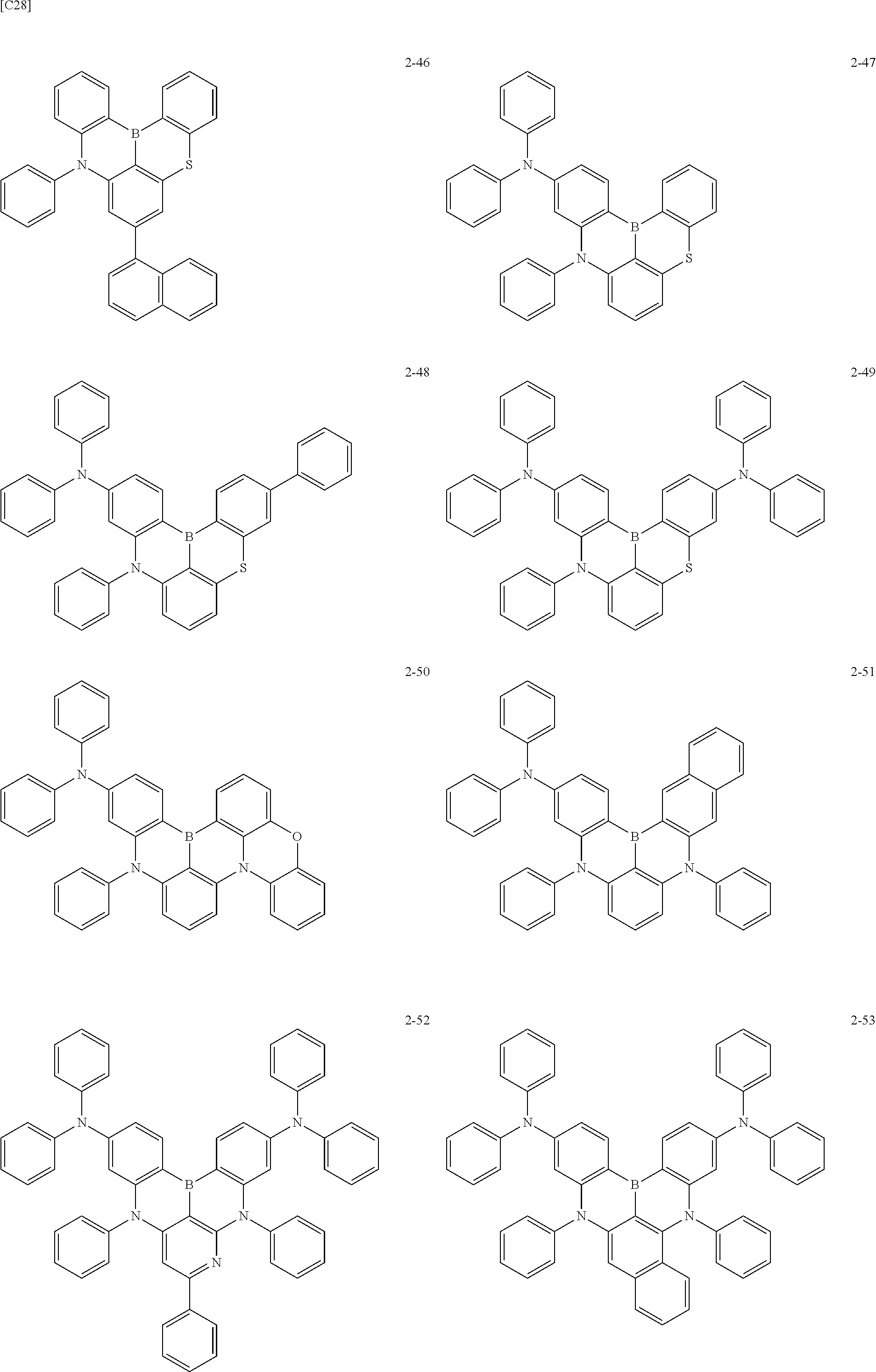

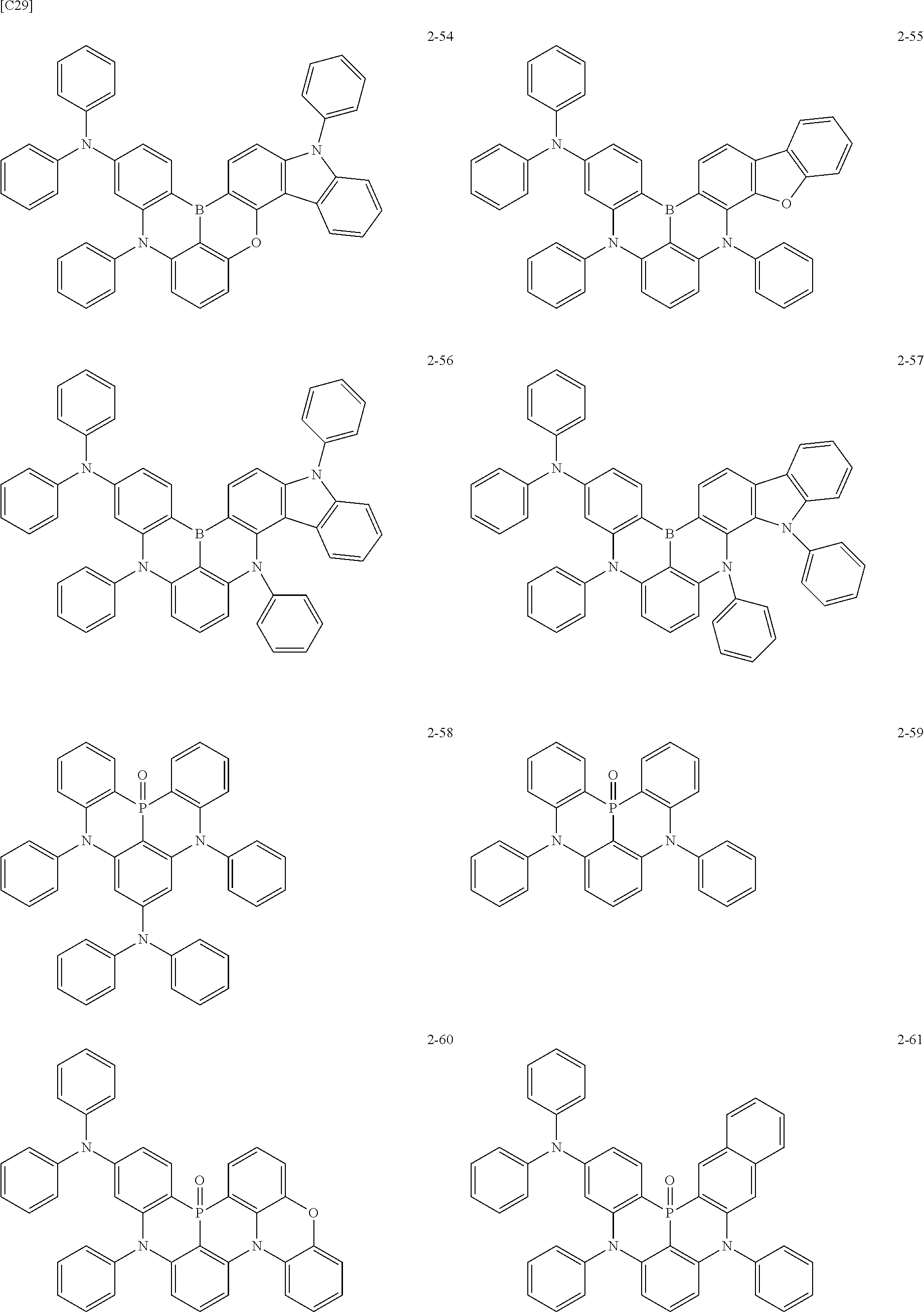

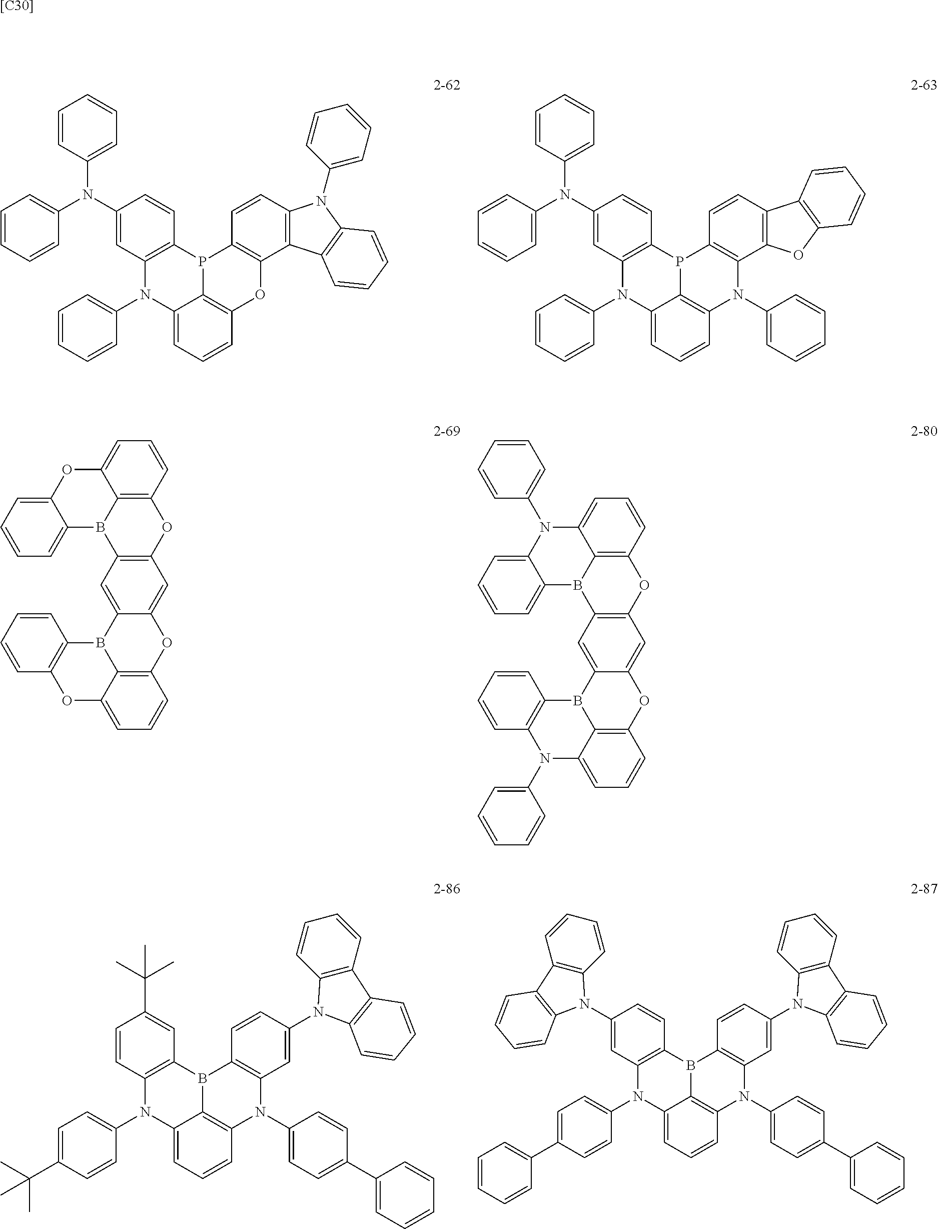

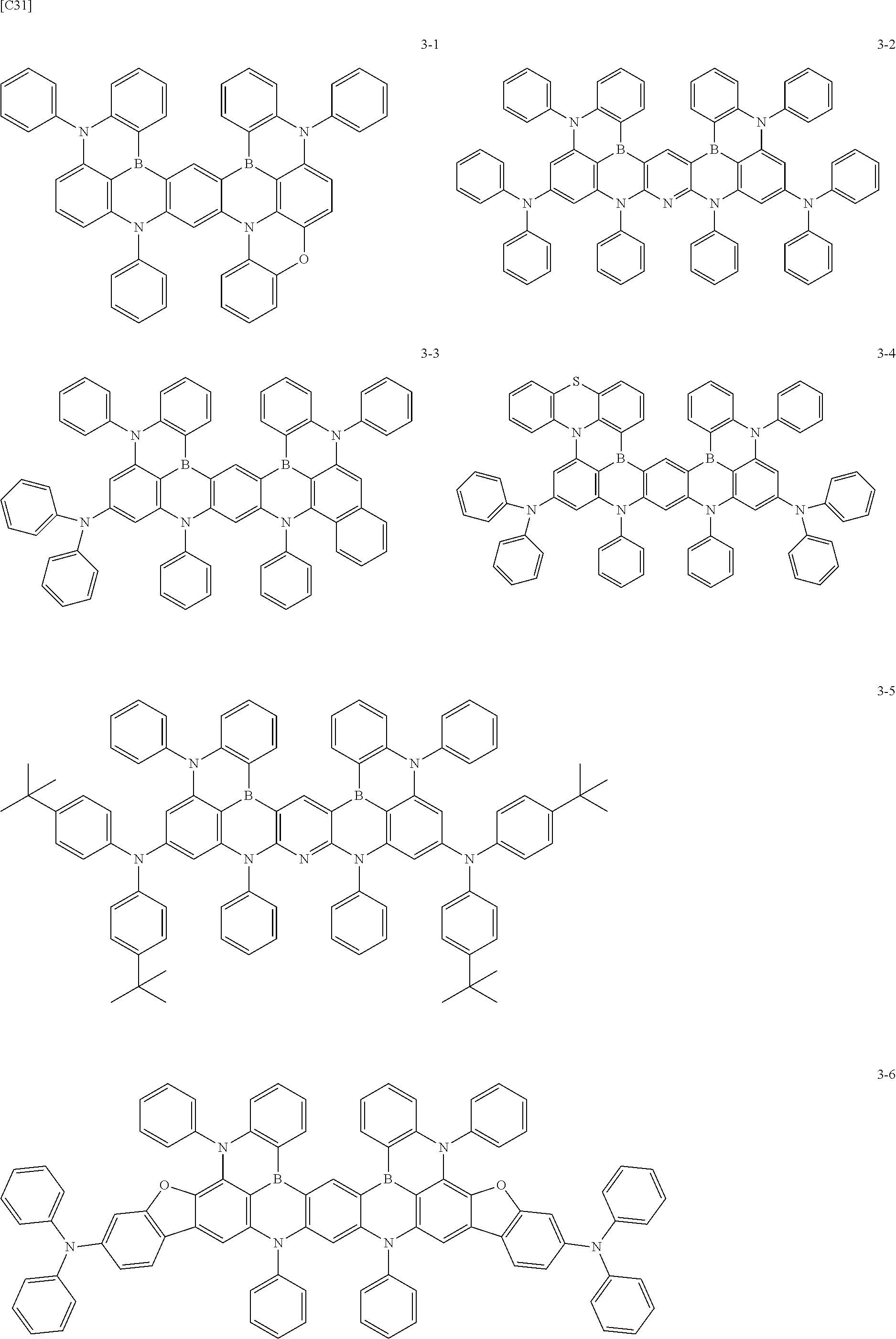

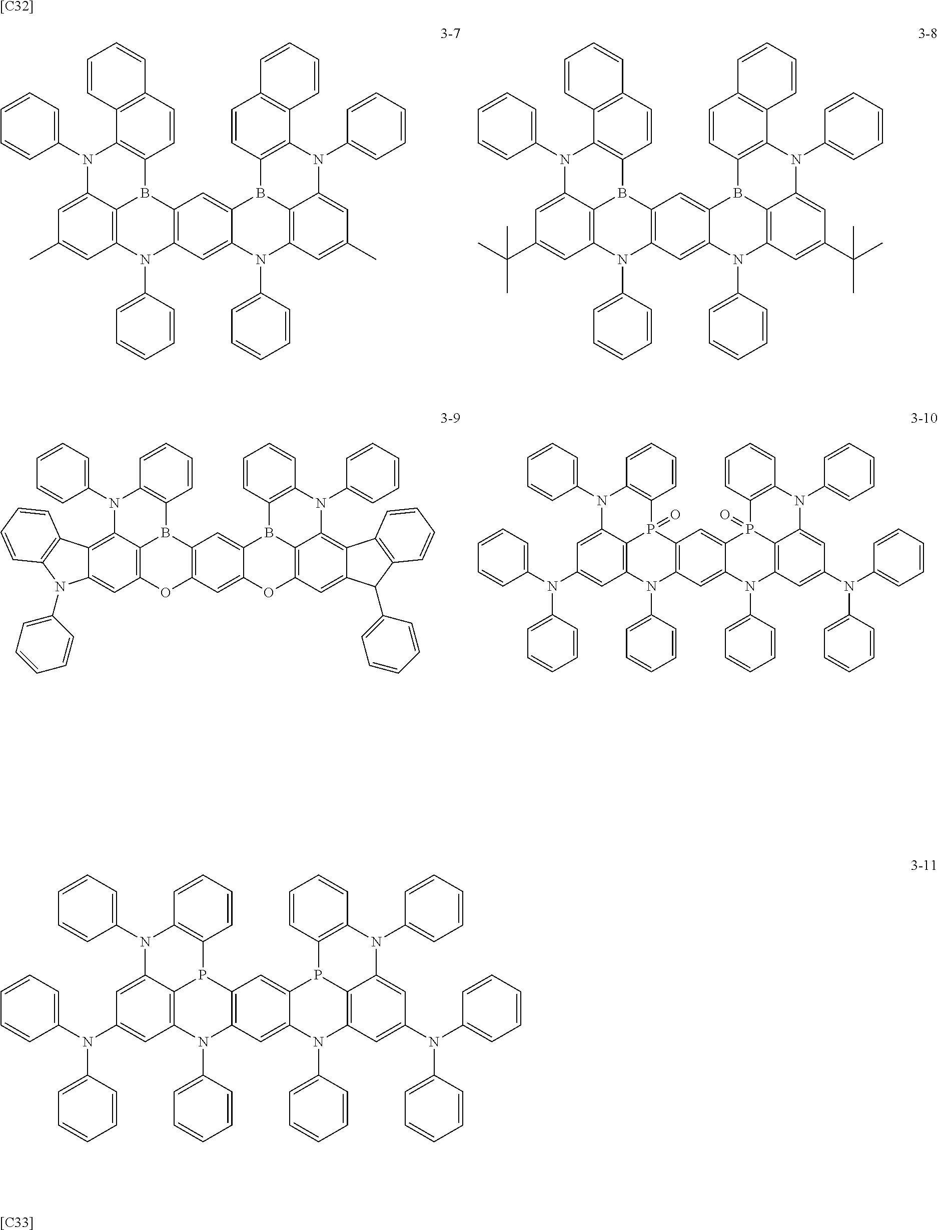

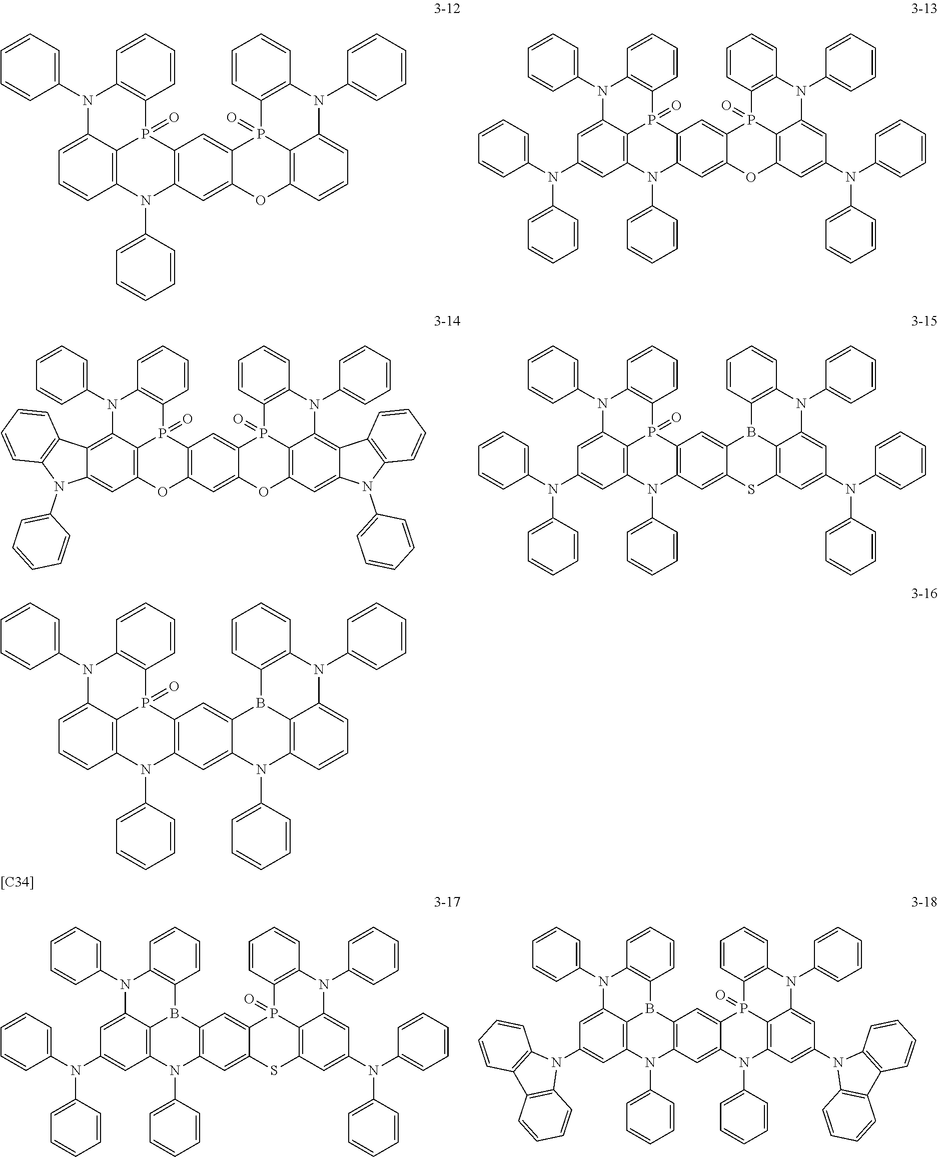

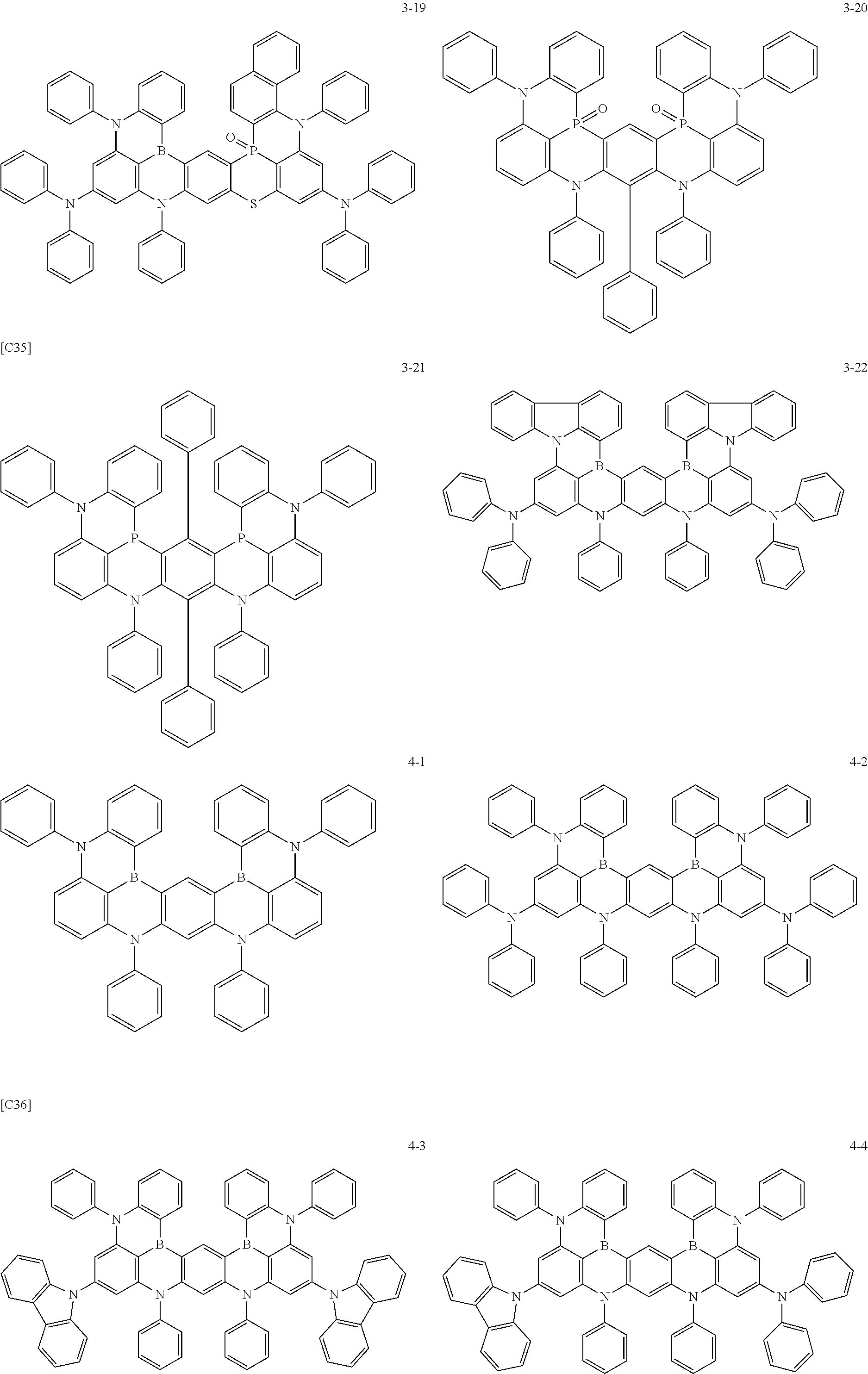

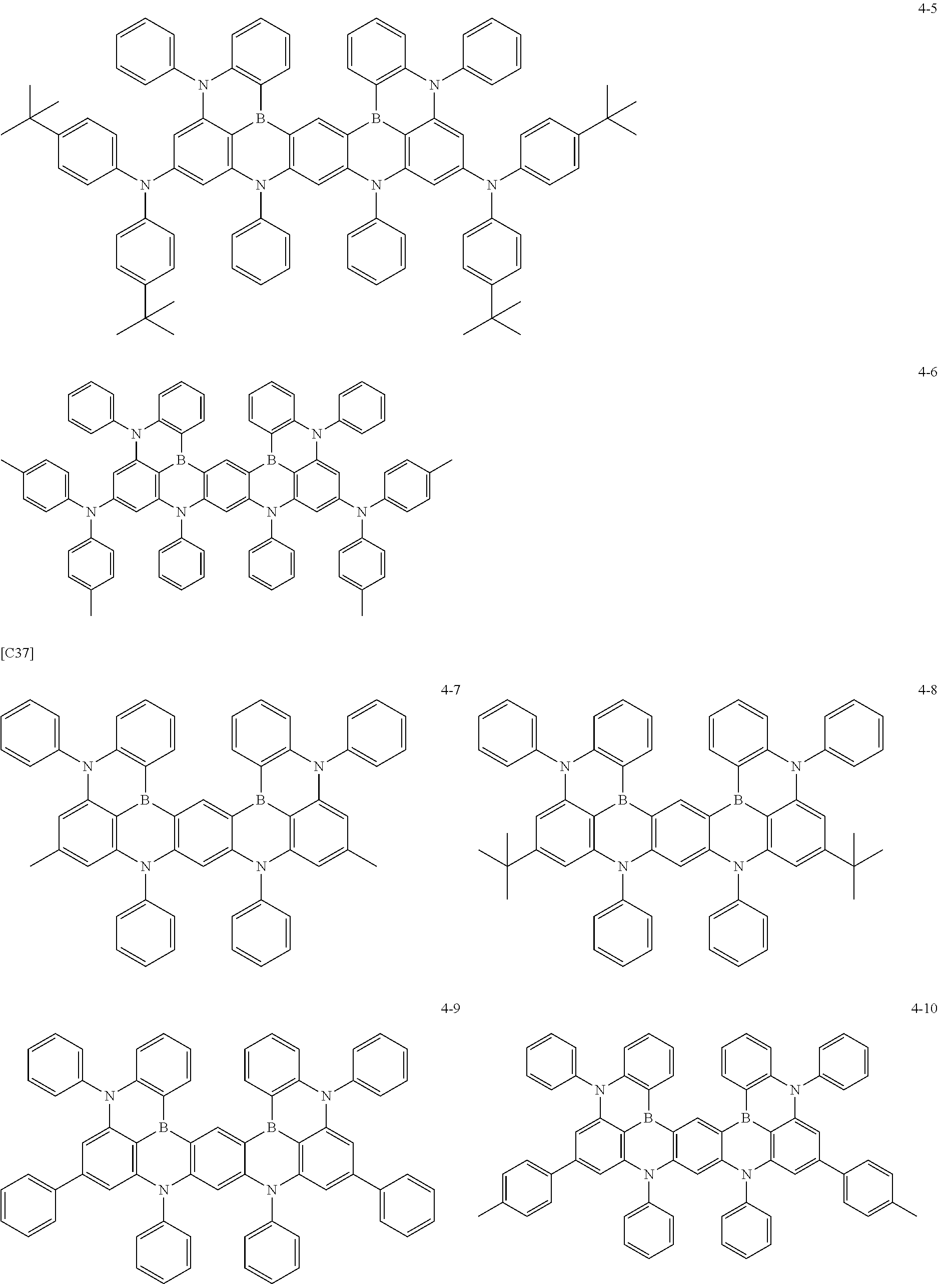

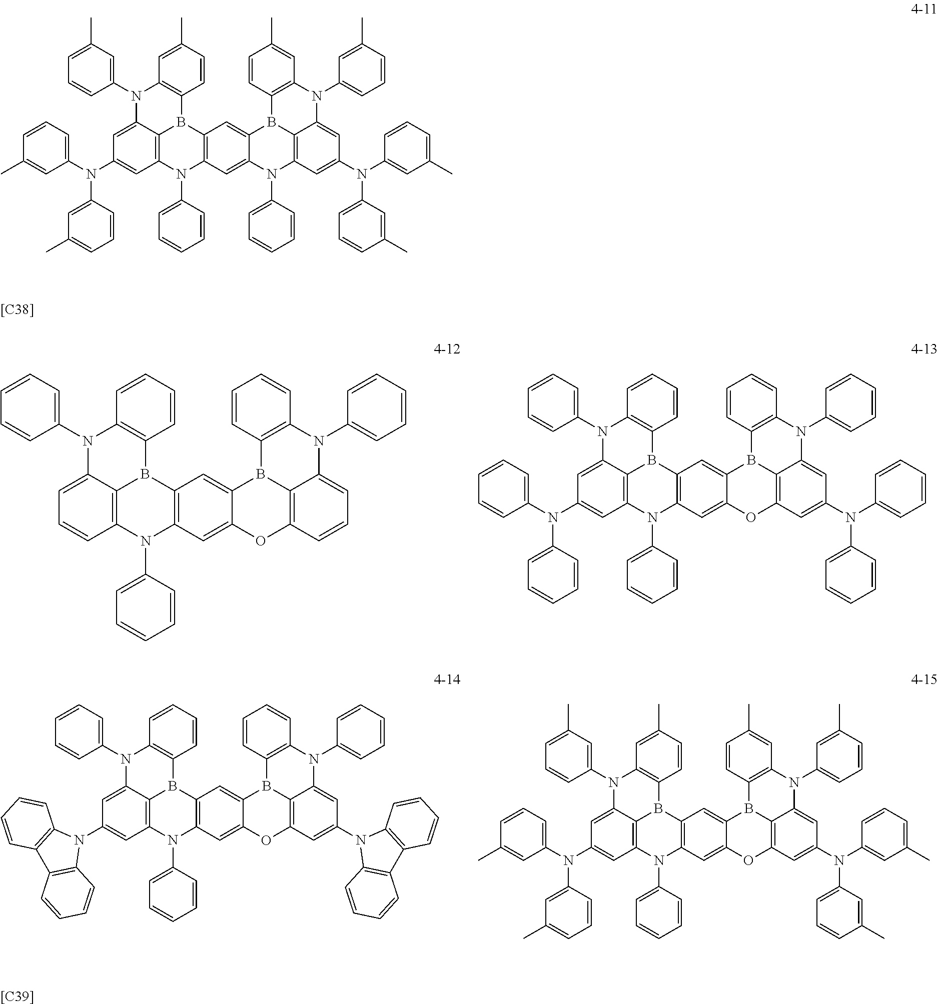

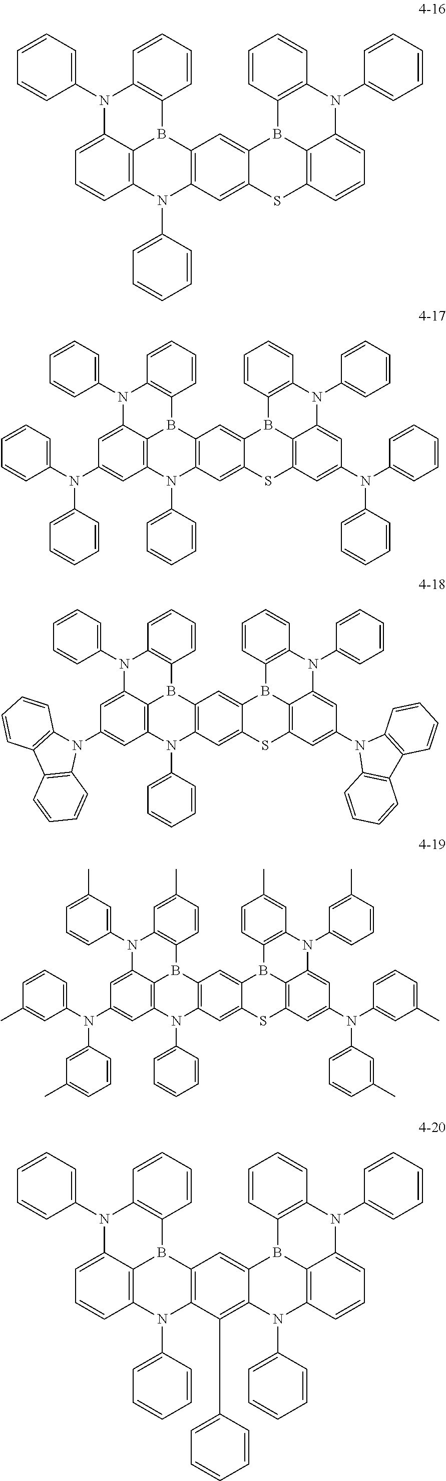

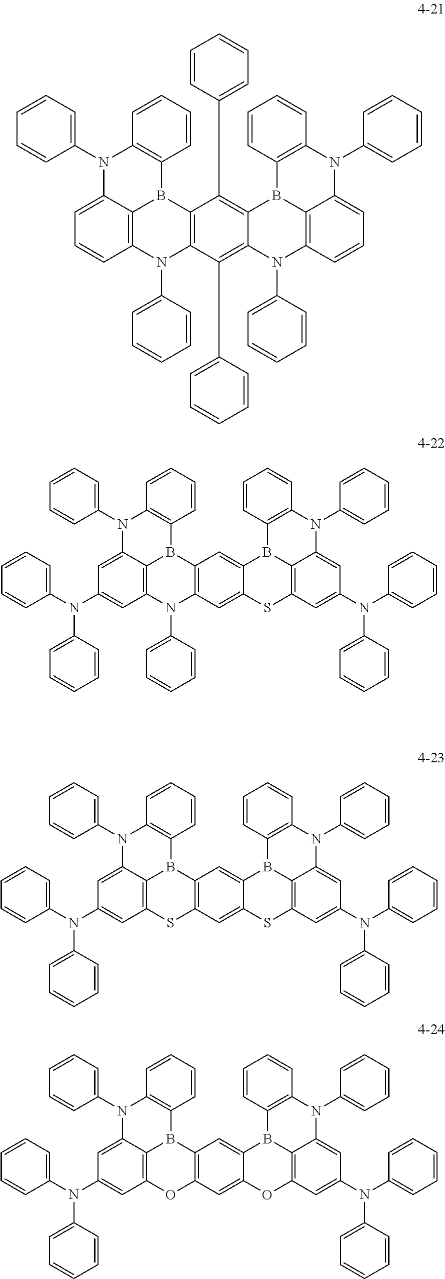

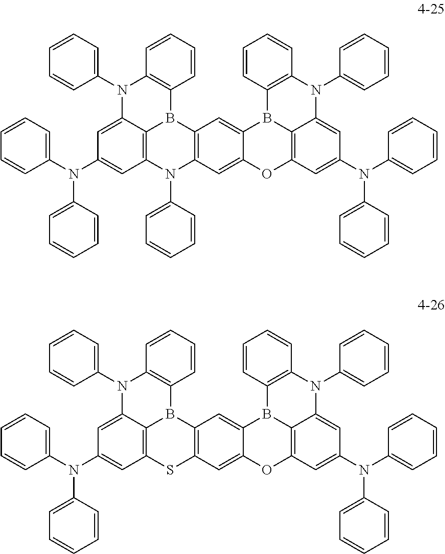

Specific examples of the compounds represented by the general formula (6) above and (6a) or (6b), which is a preferred aspect of the general formula (6) above, will be described below, but are not limited to these exemplary compounds.

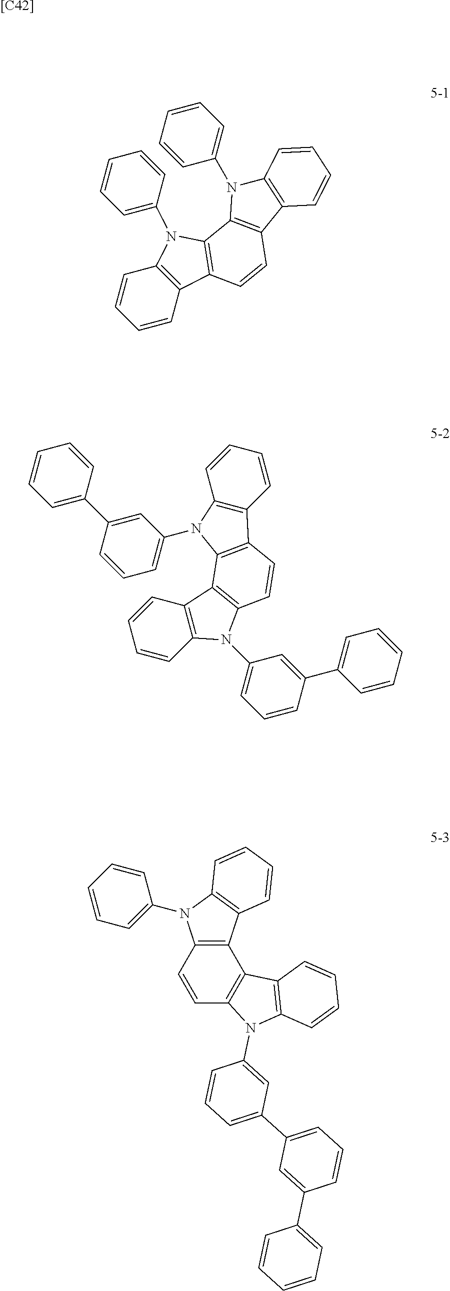

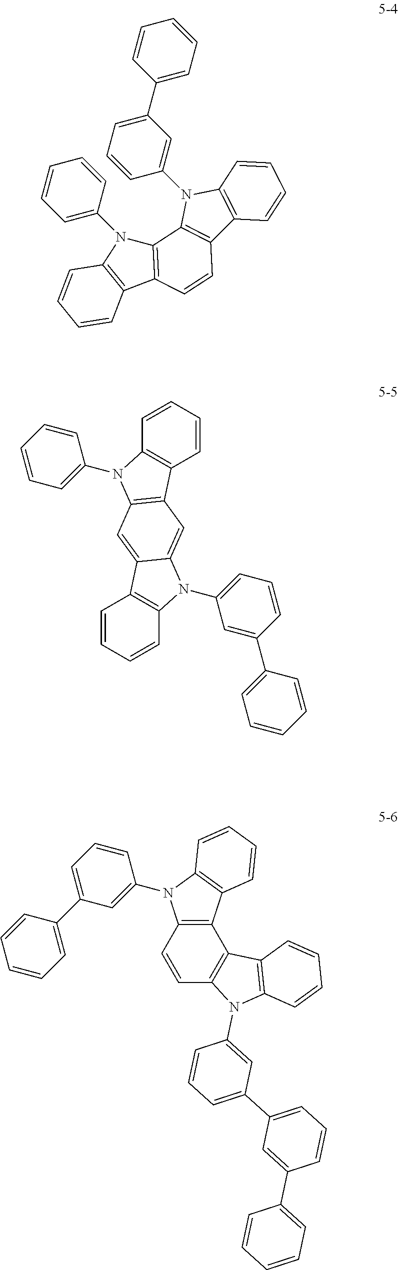

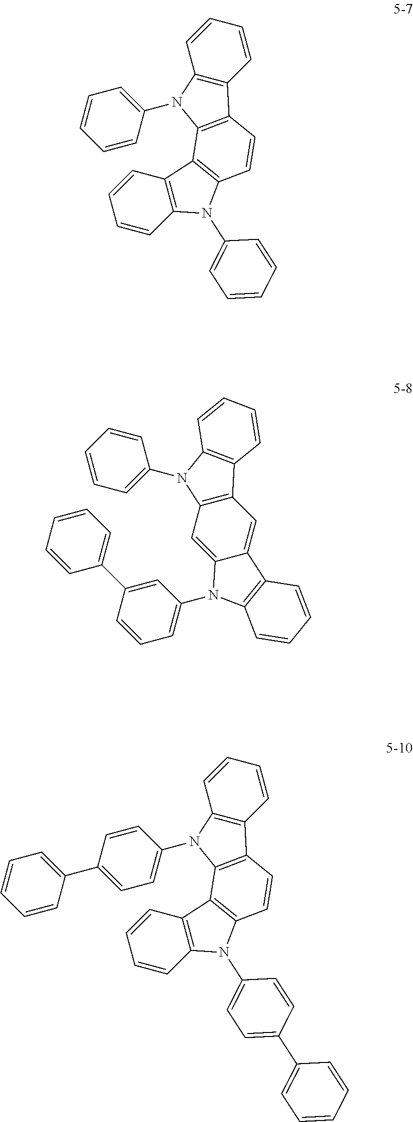

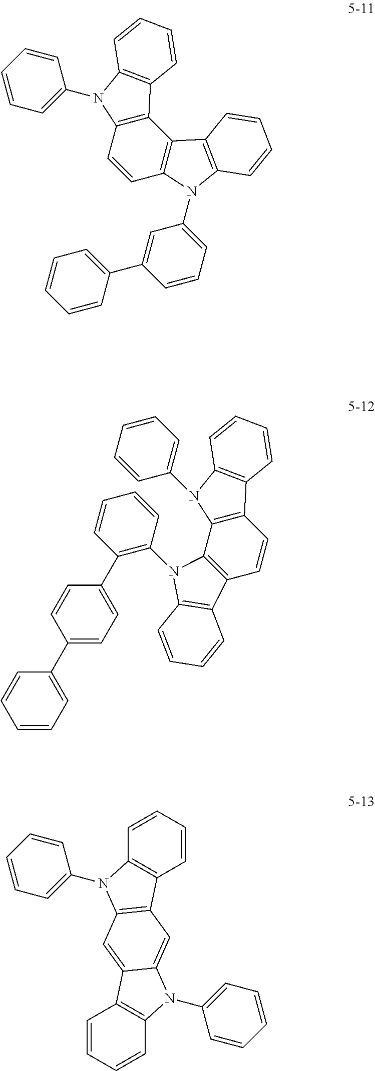

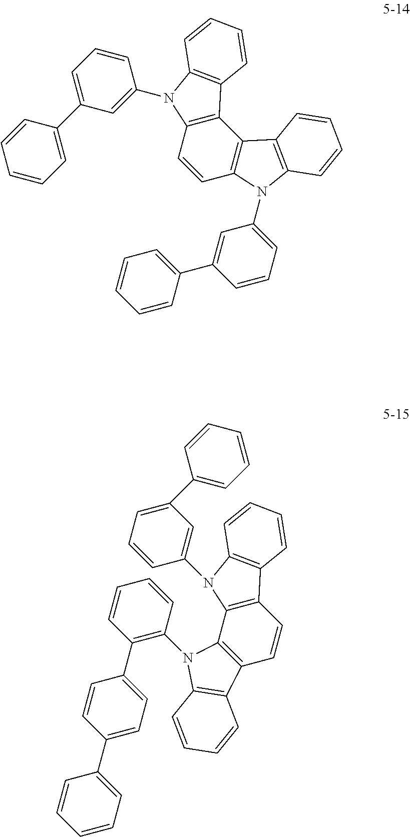

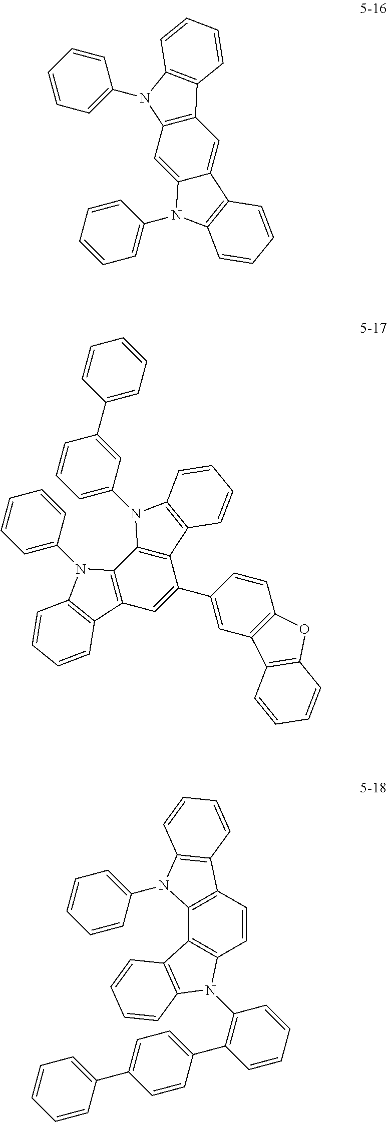

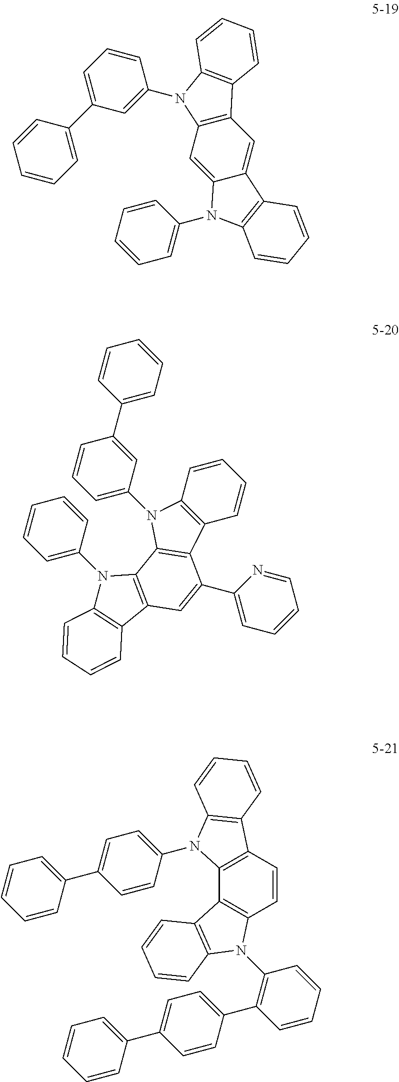

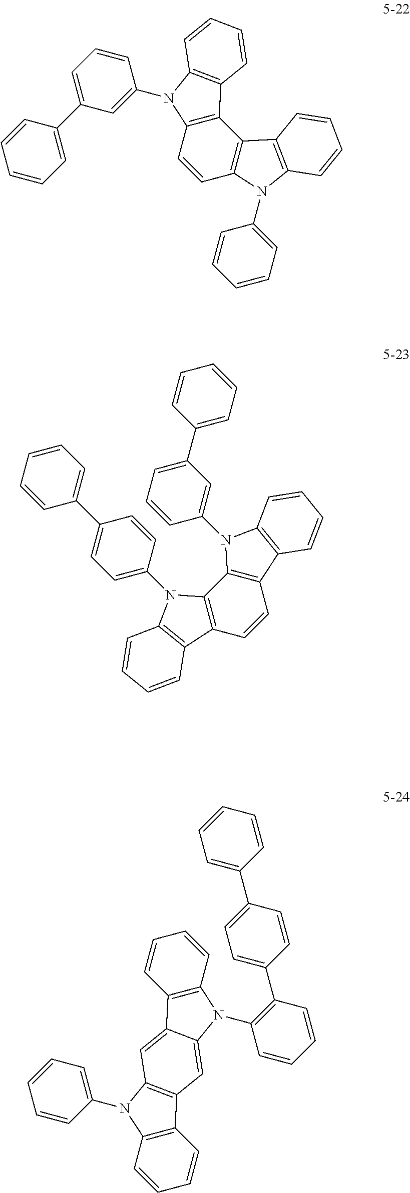

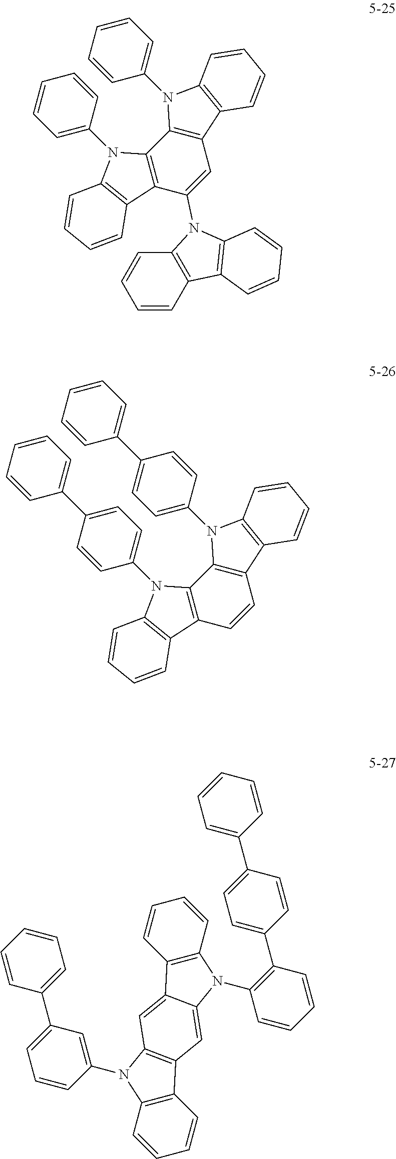

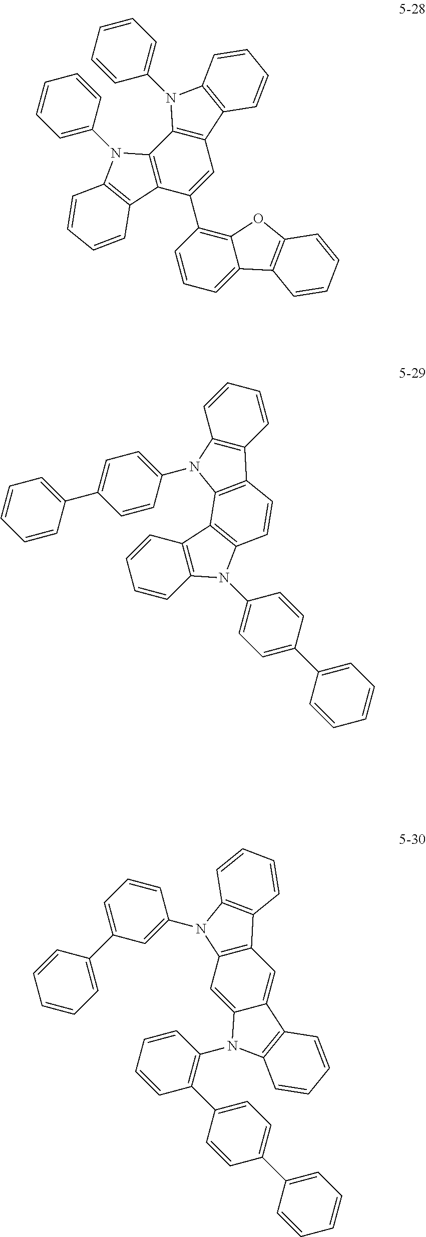

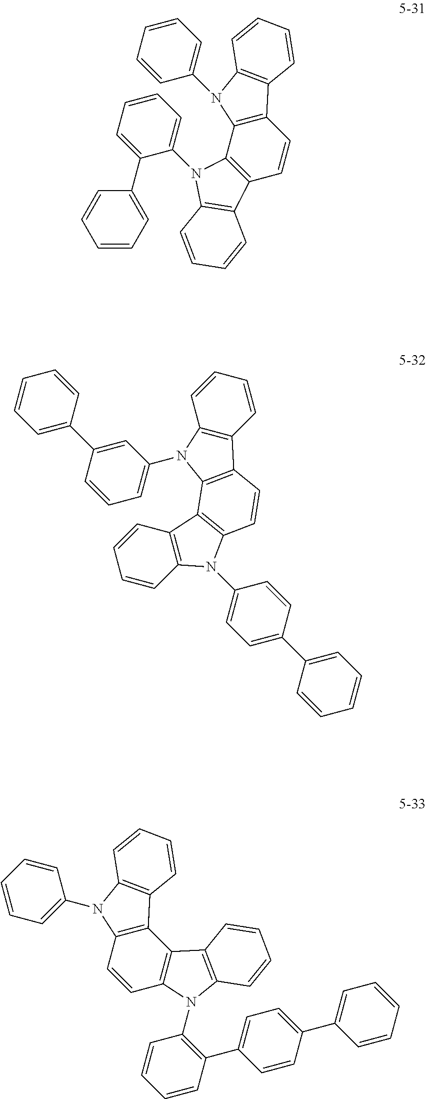

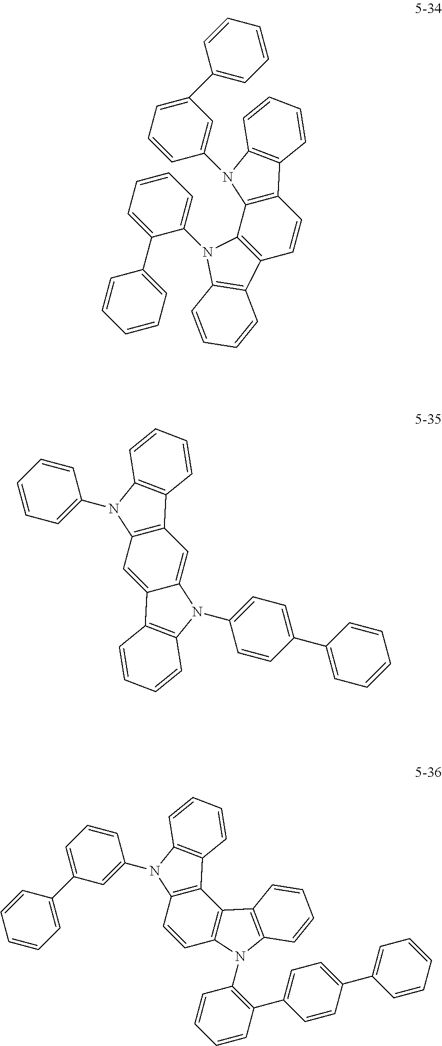

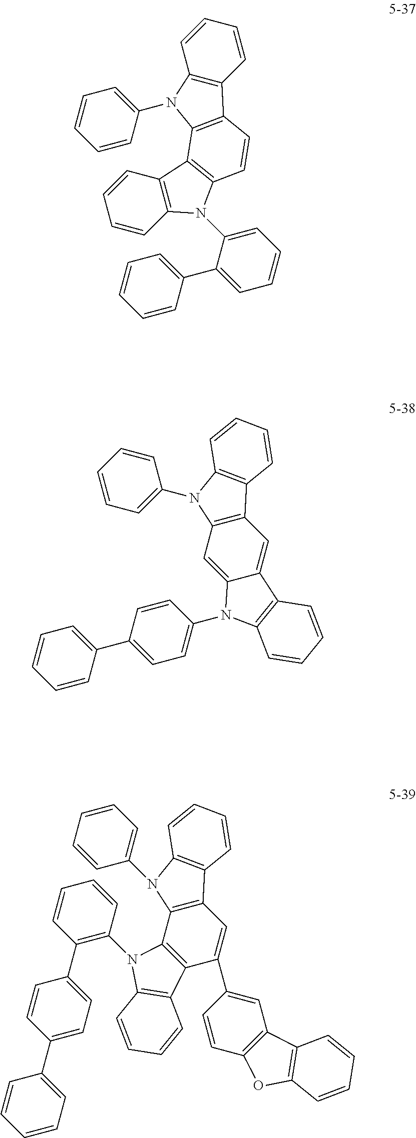

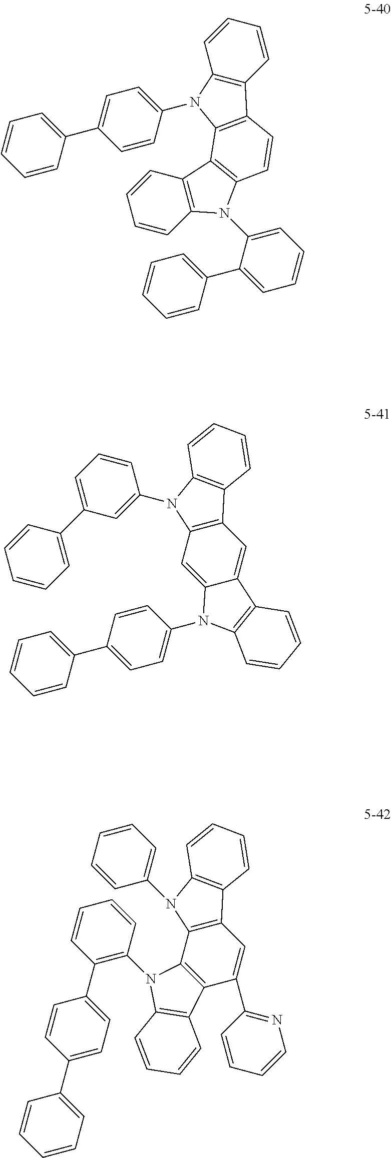

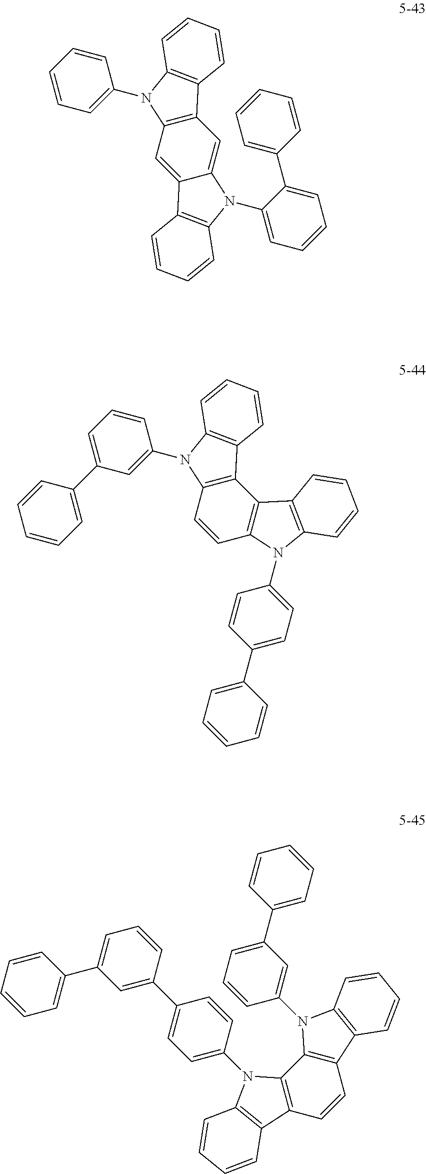

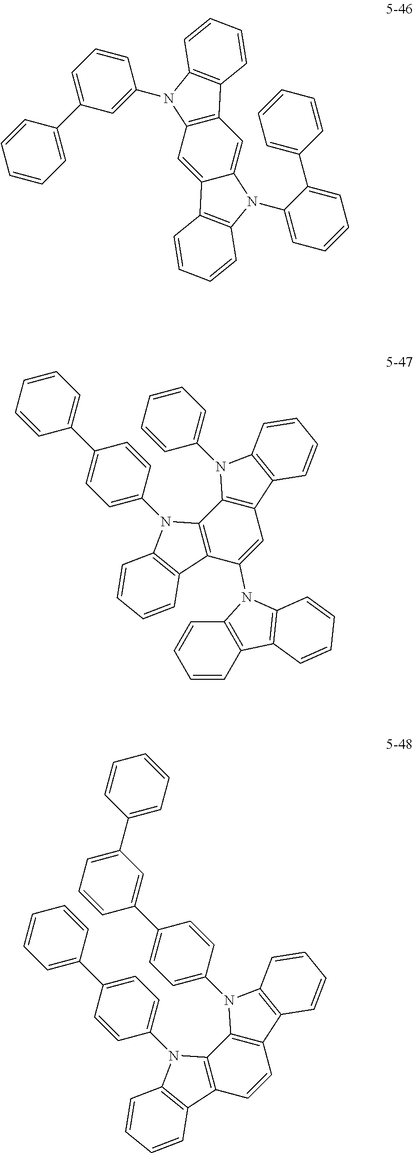

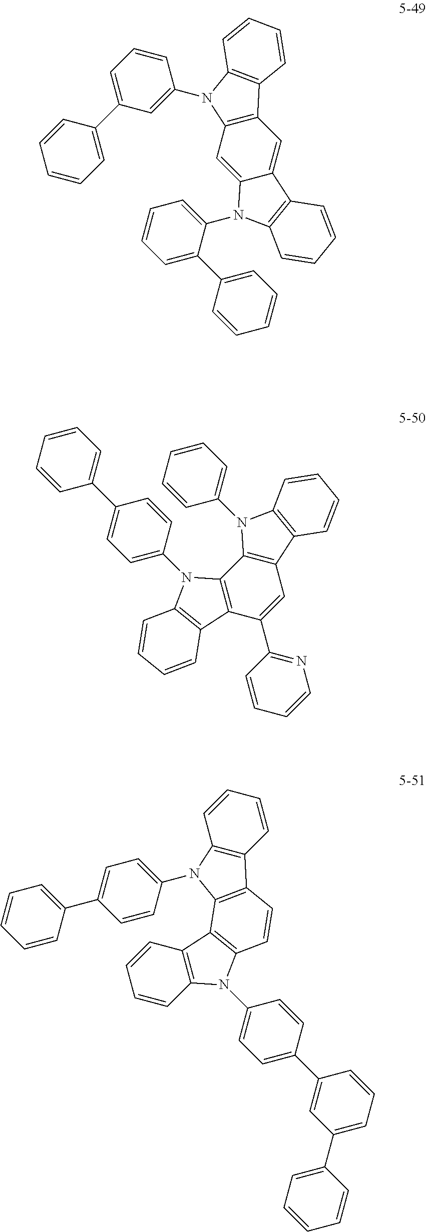

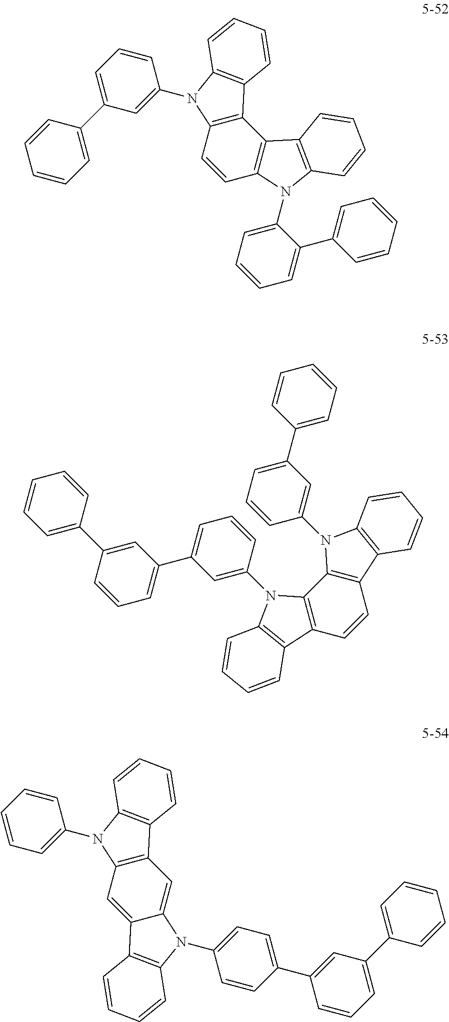

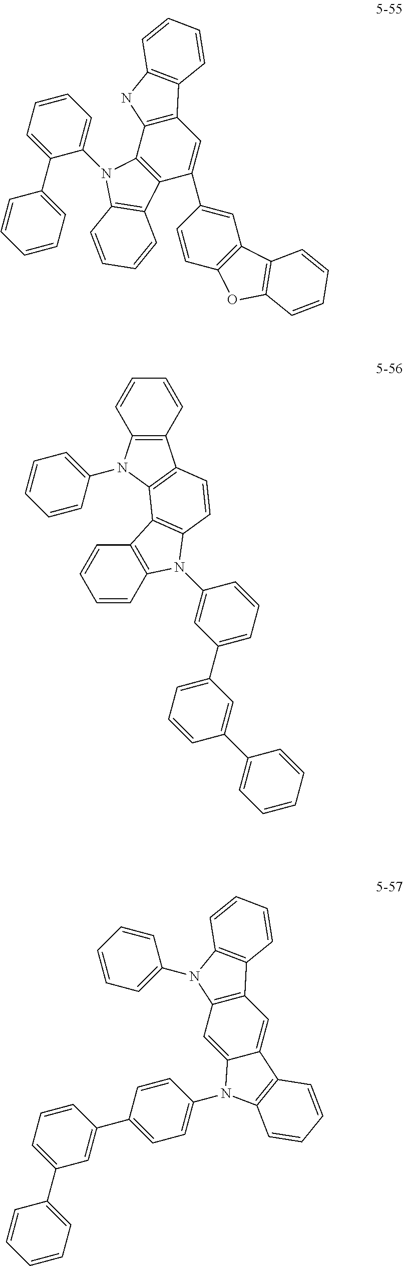

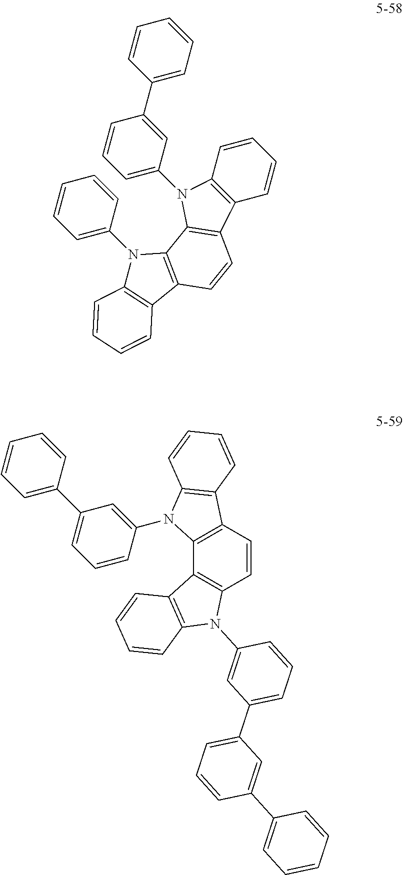

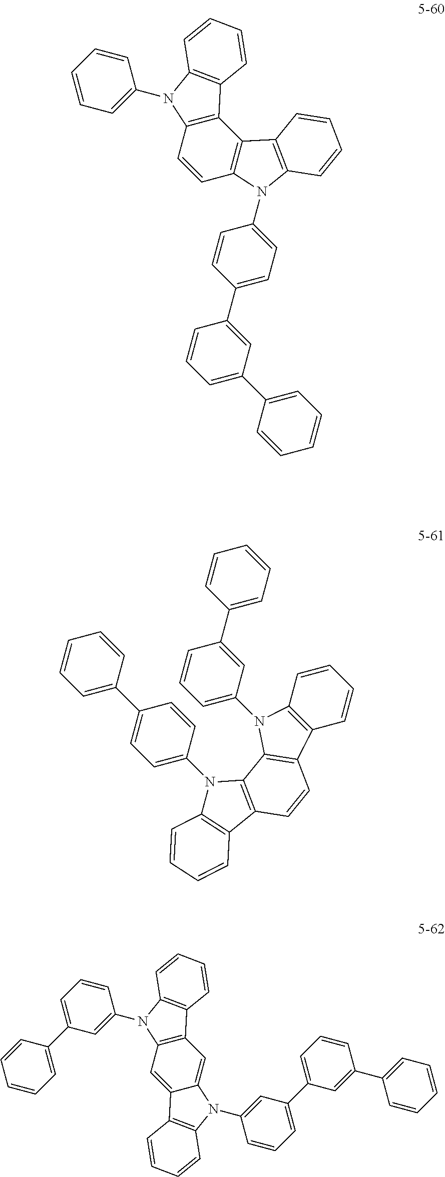

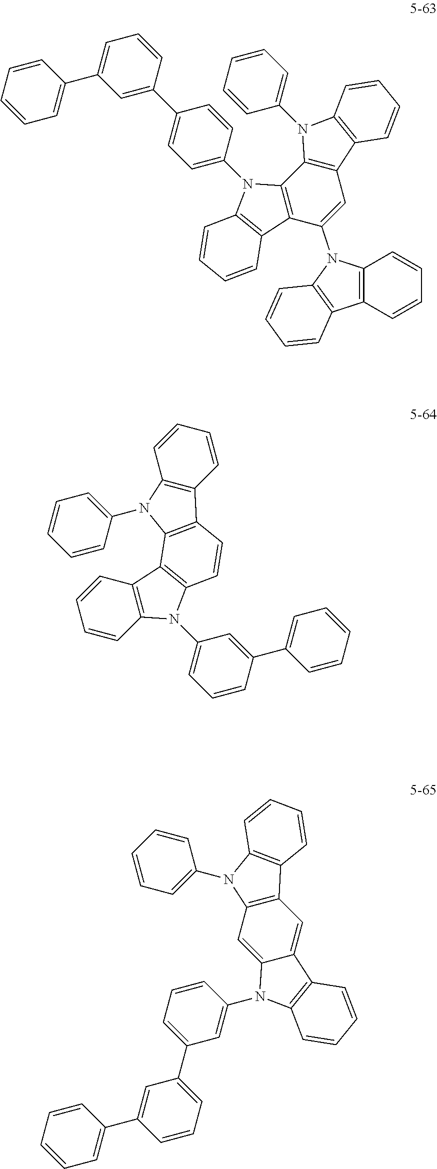

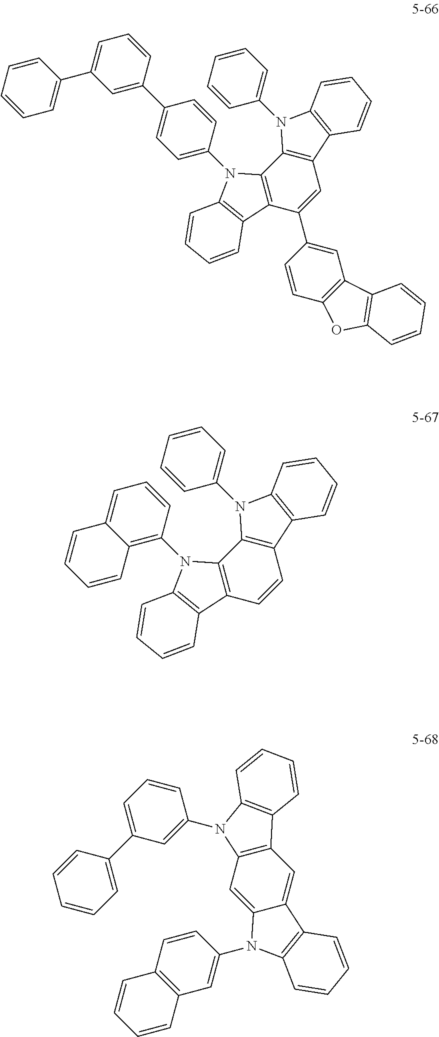

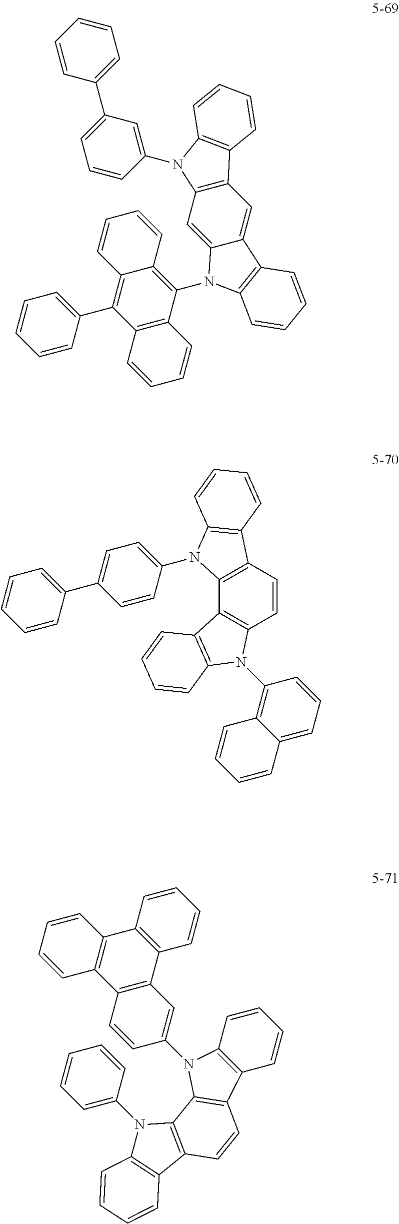

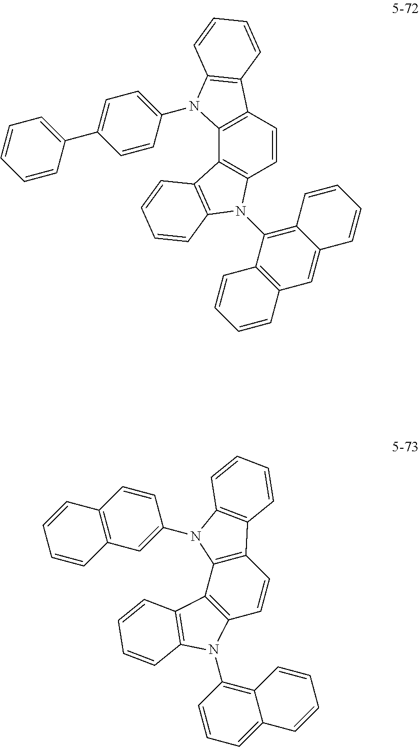

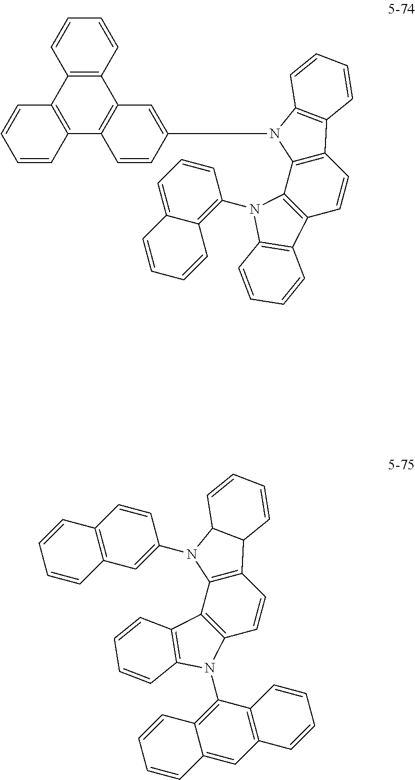

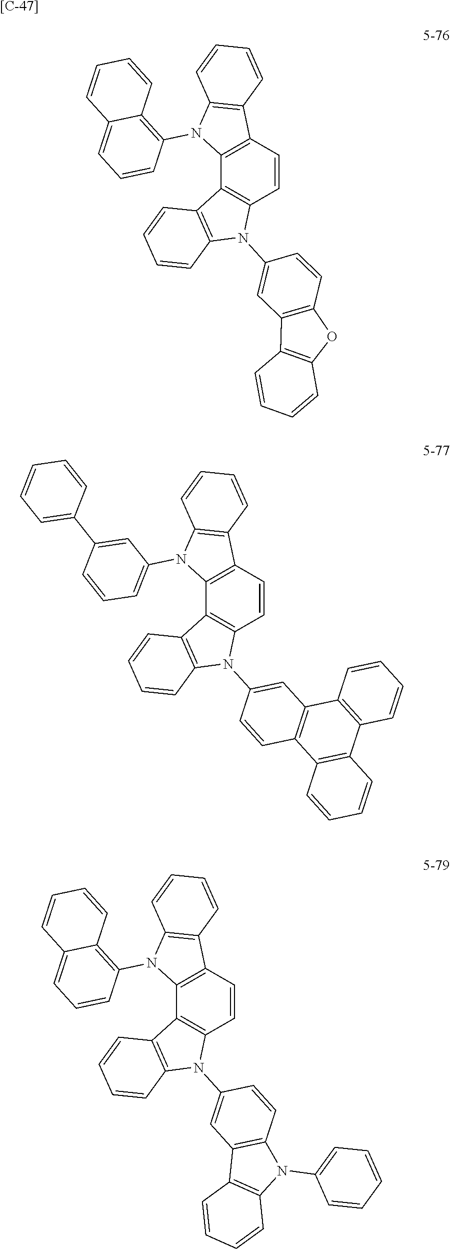

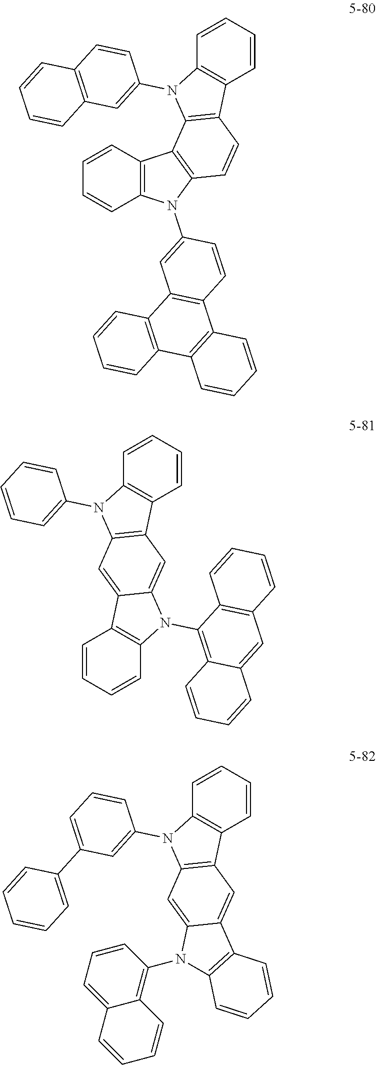

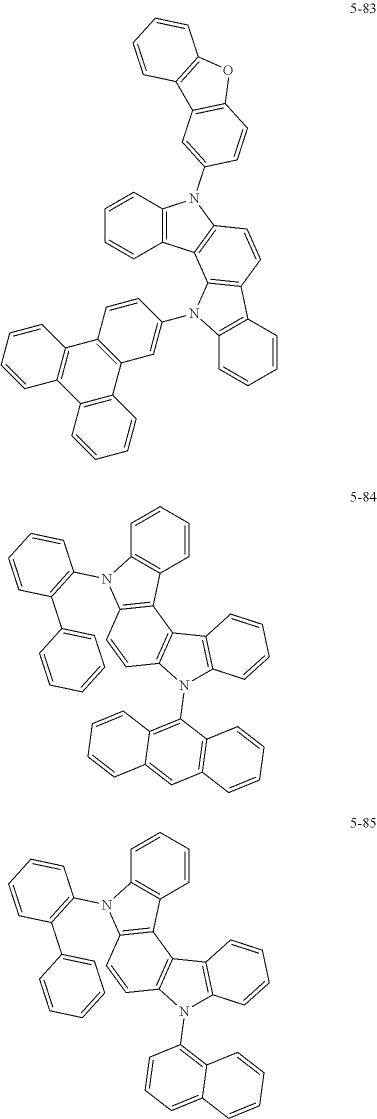

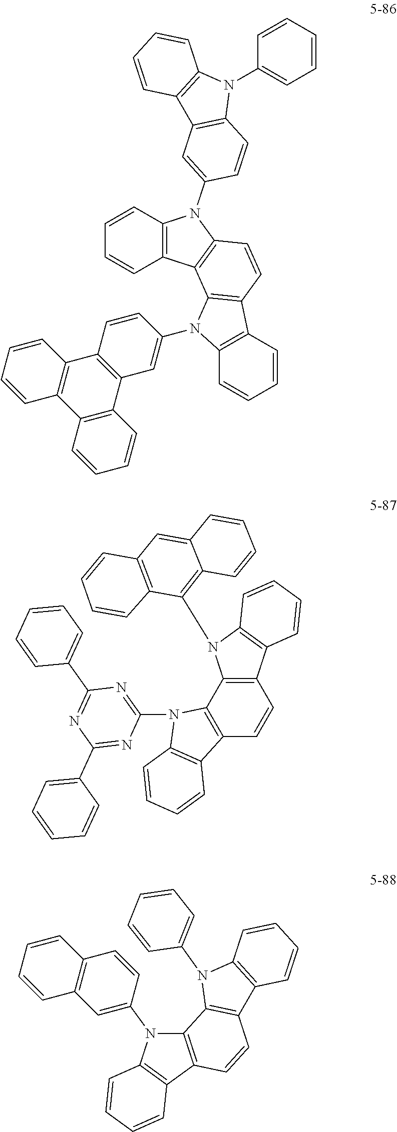

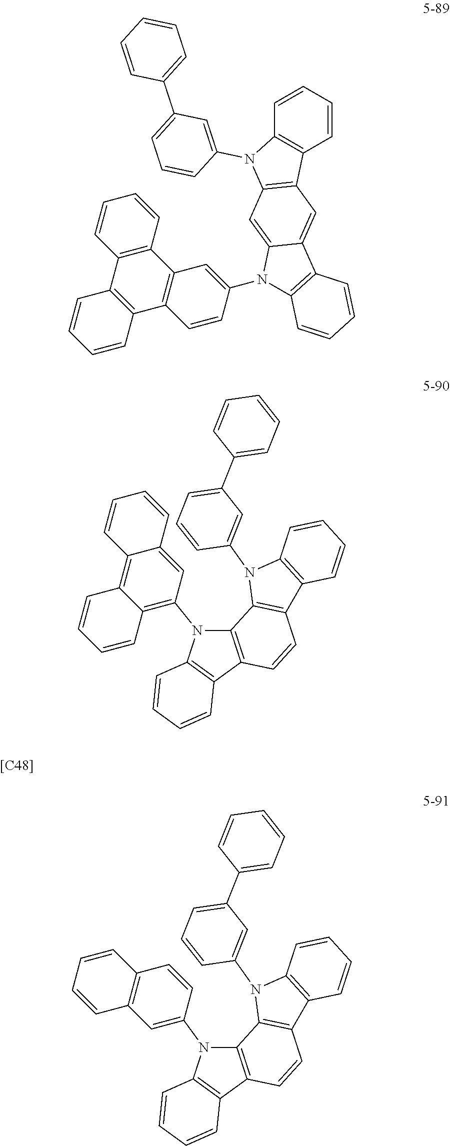

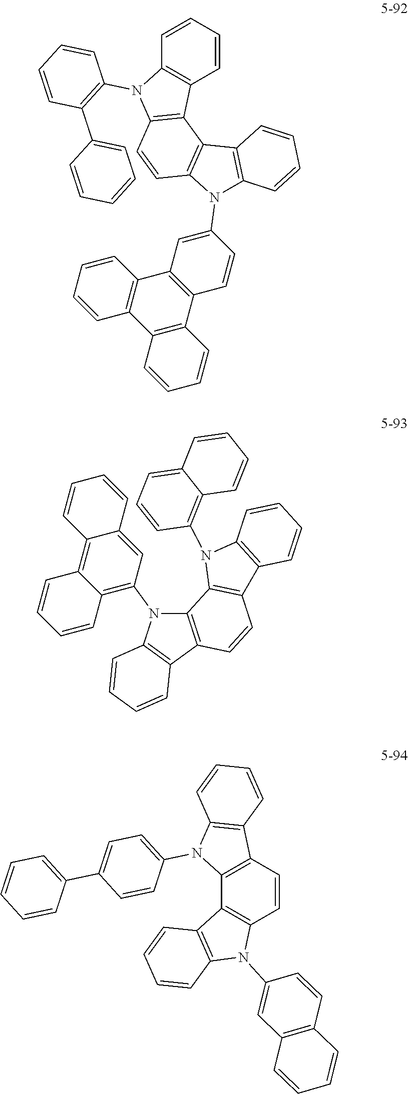

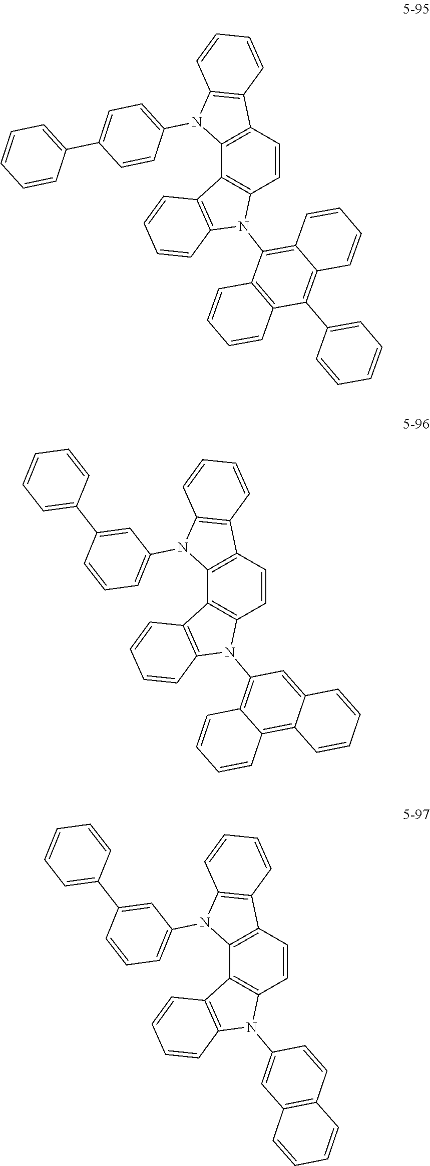

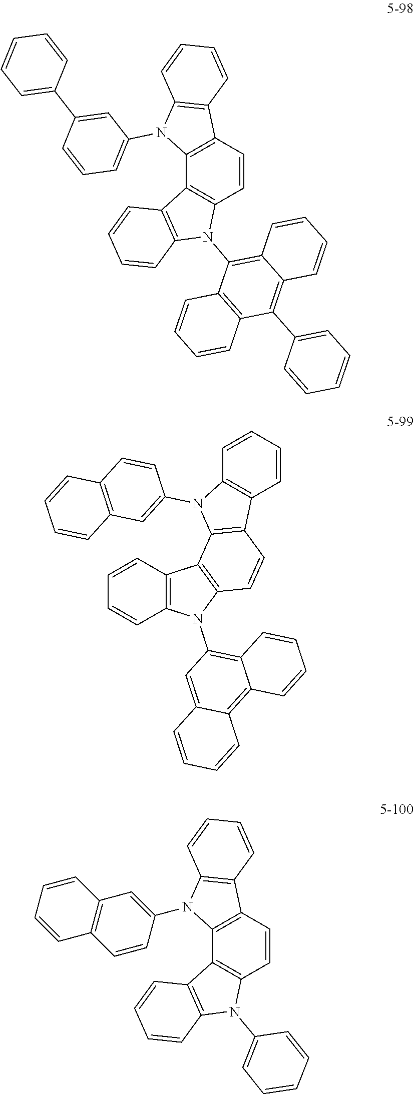

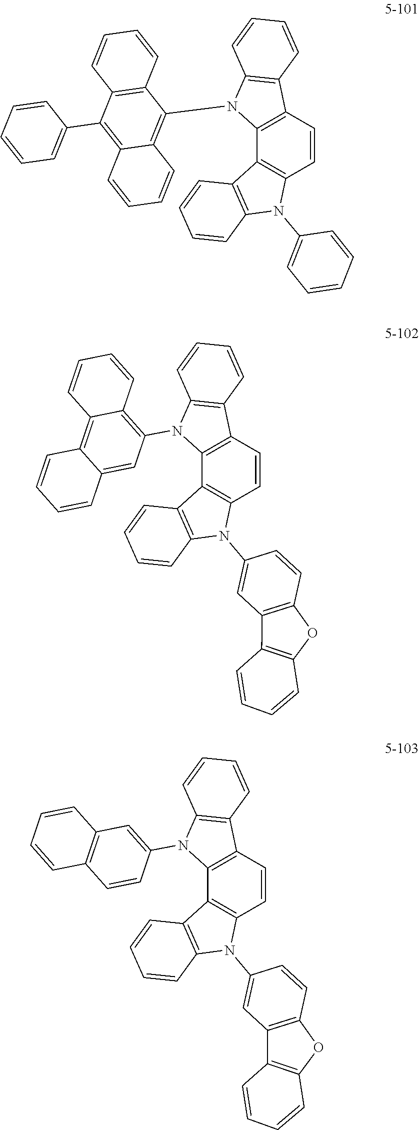

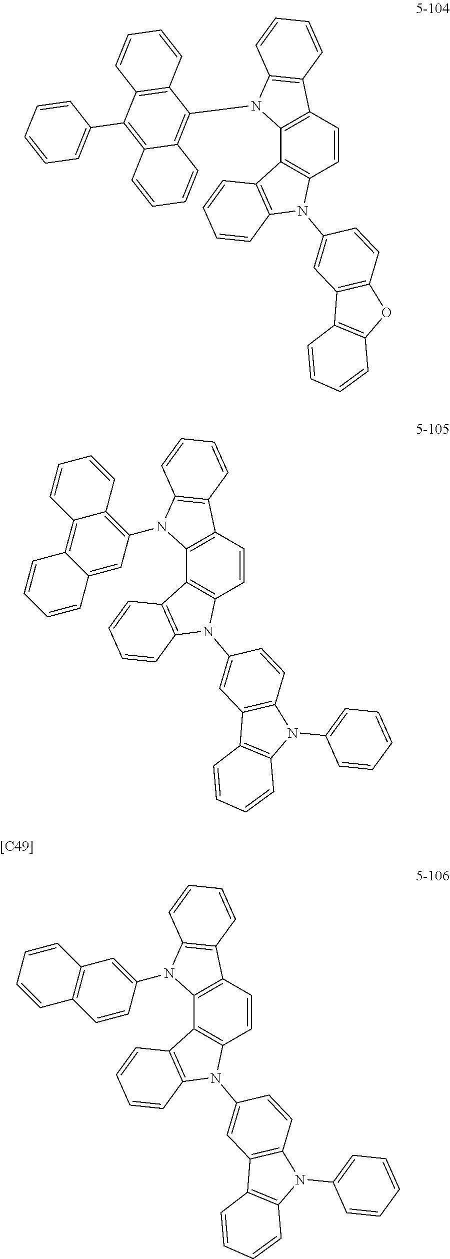

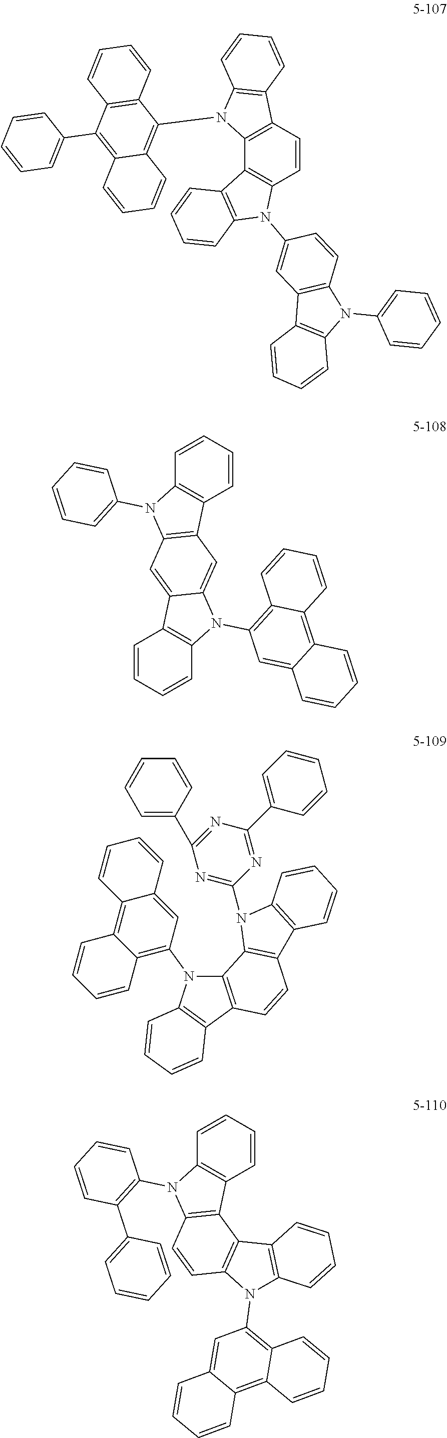

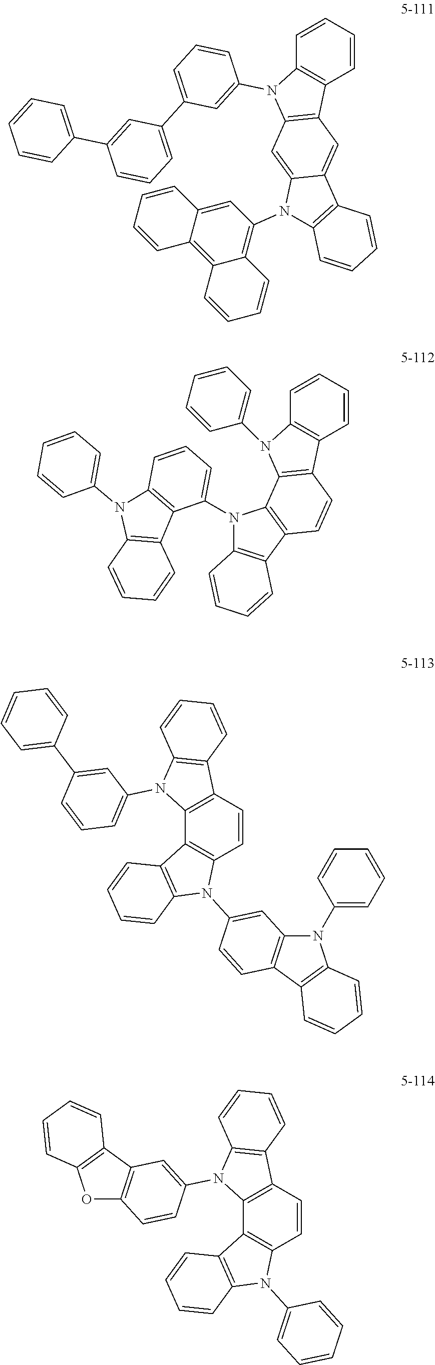

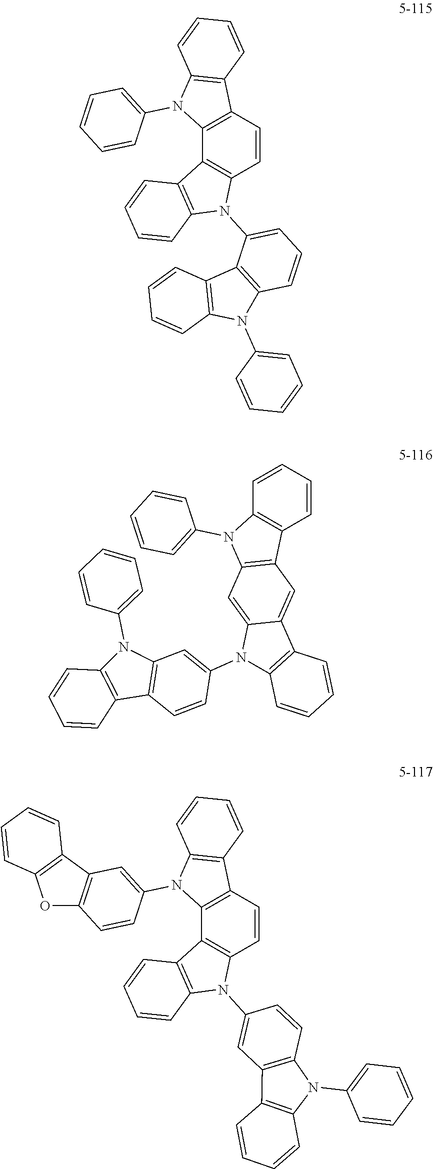

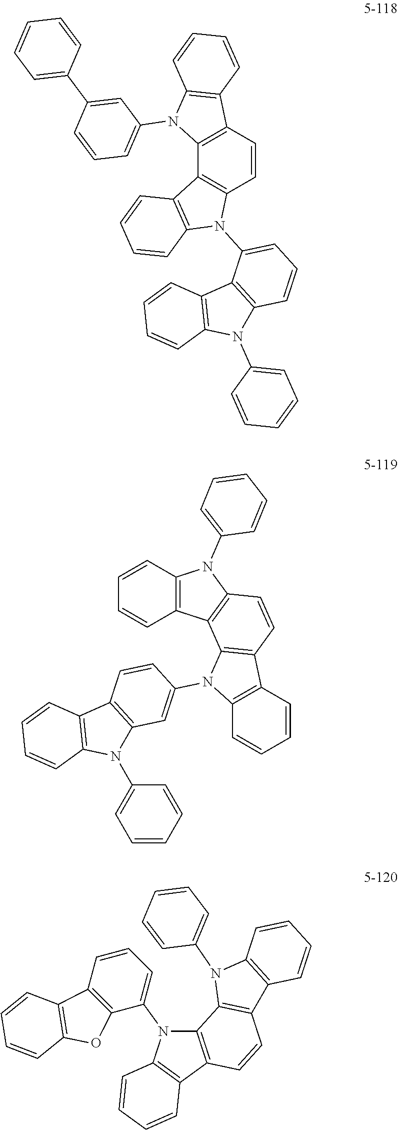

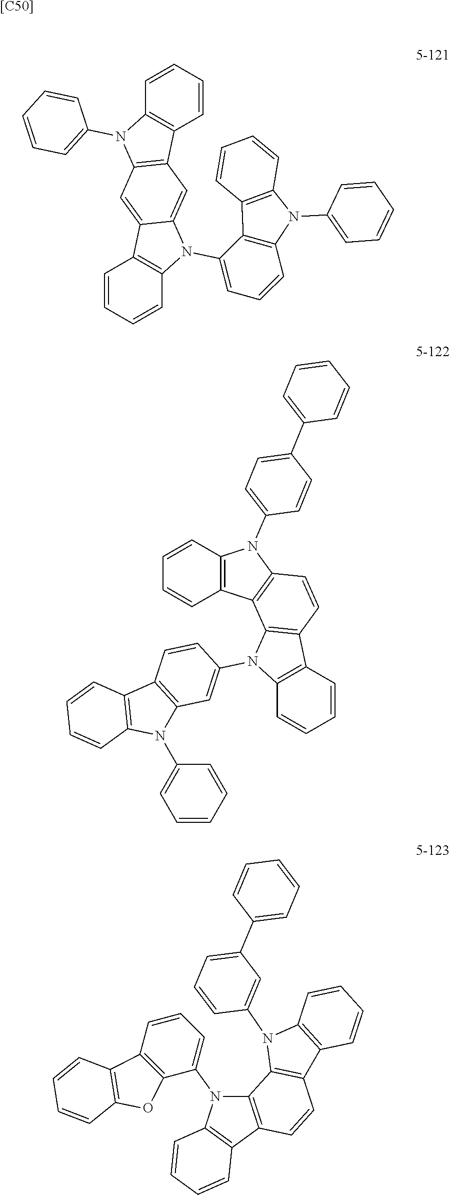

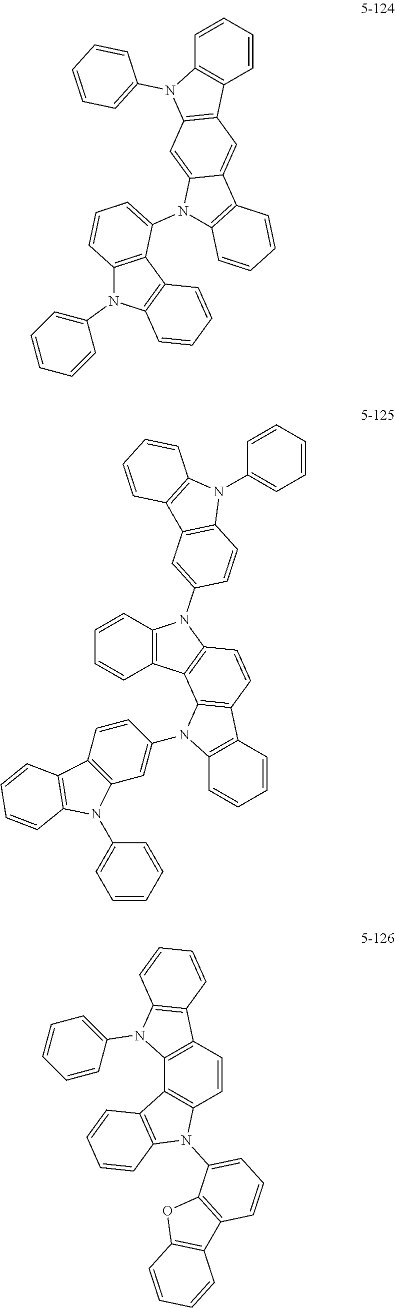

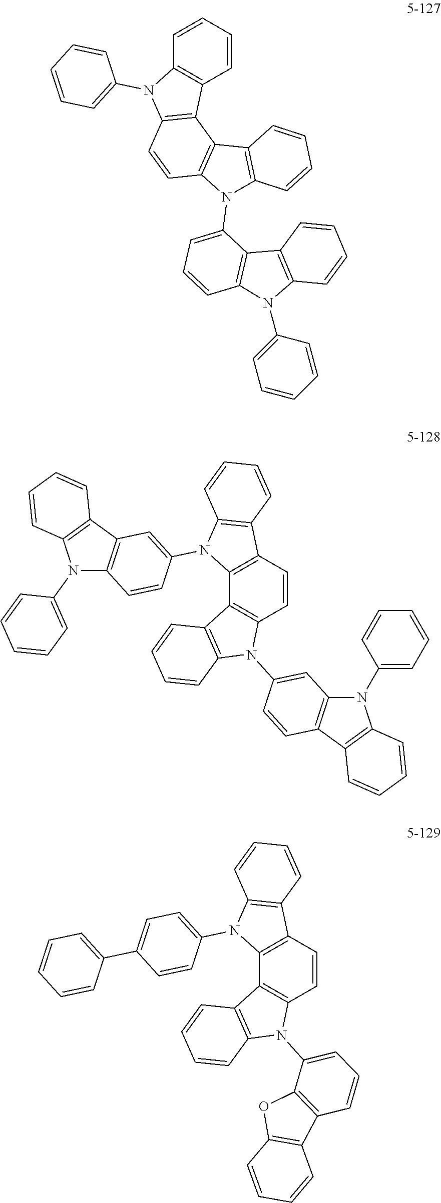

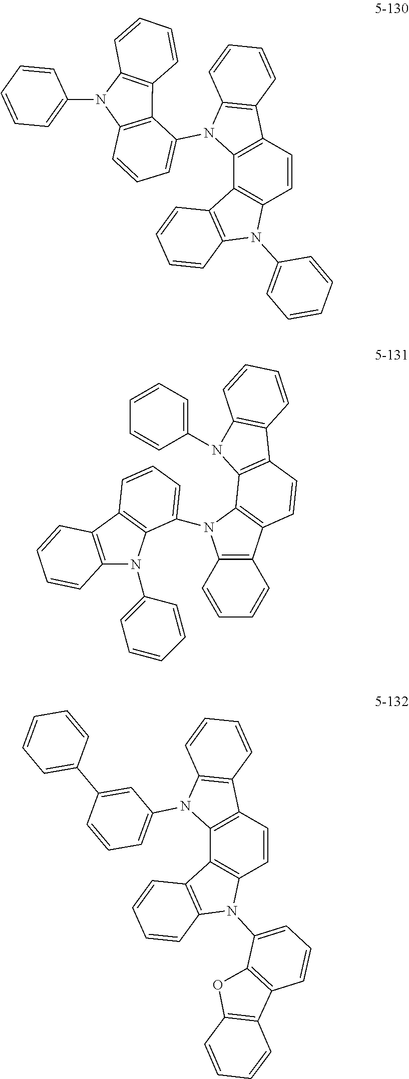

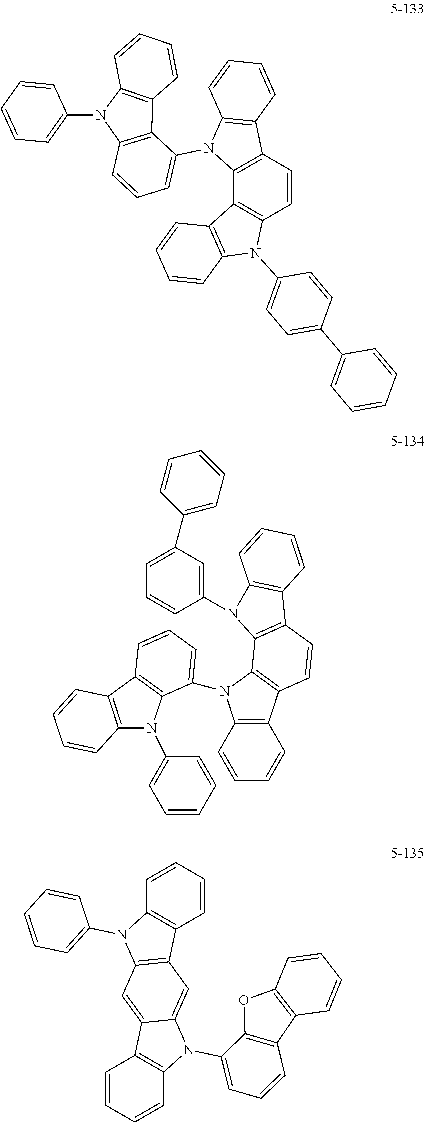

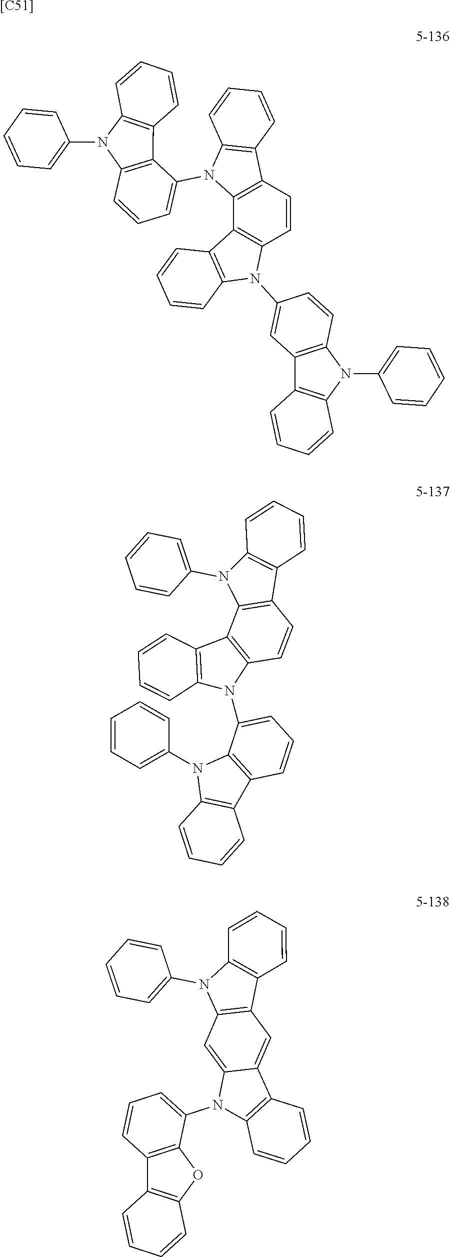

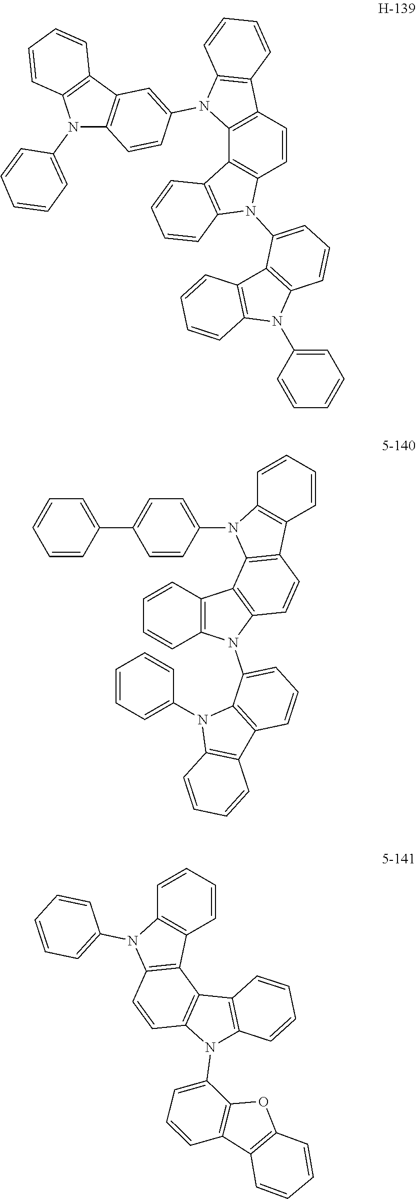

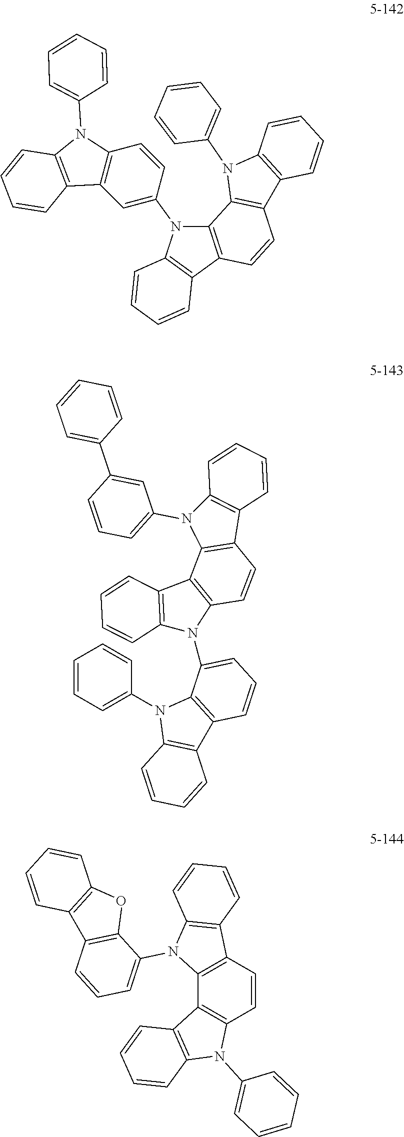

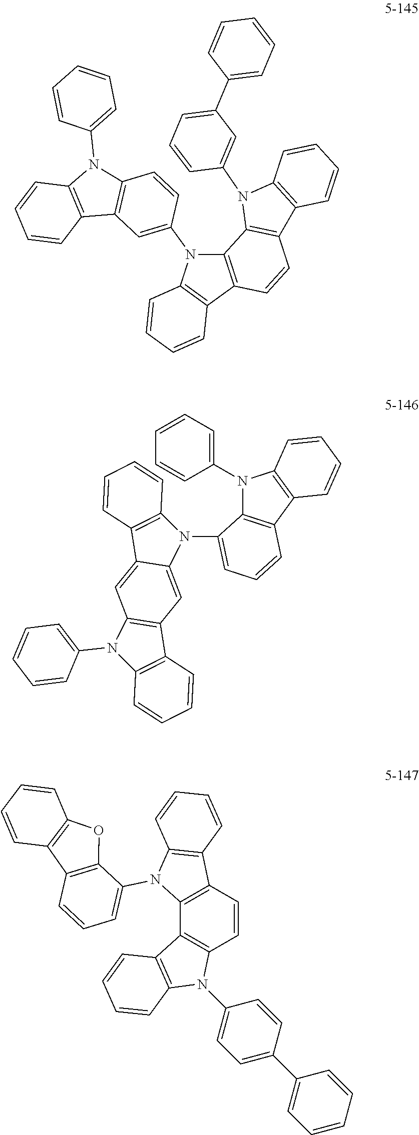

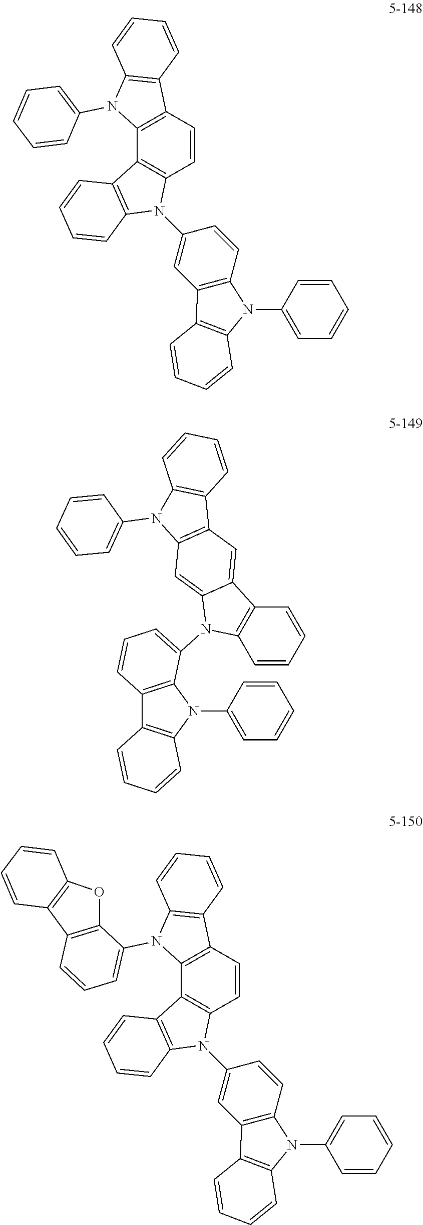

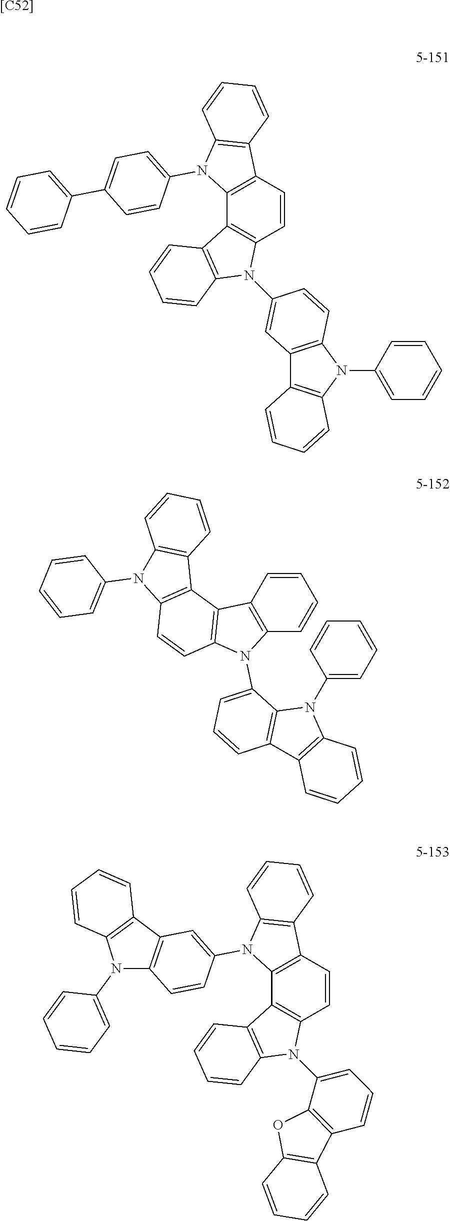

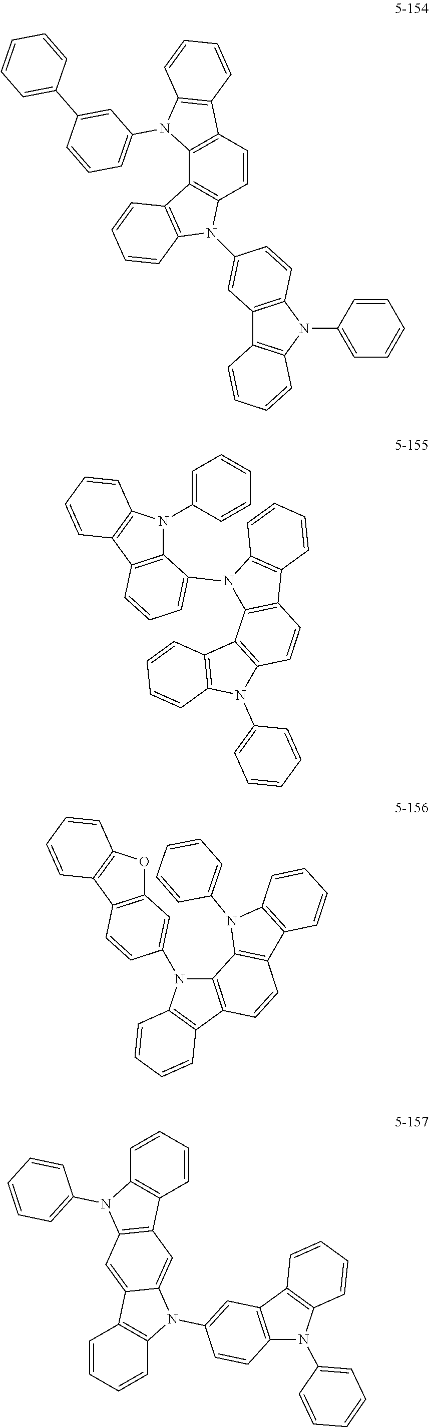

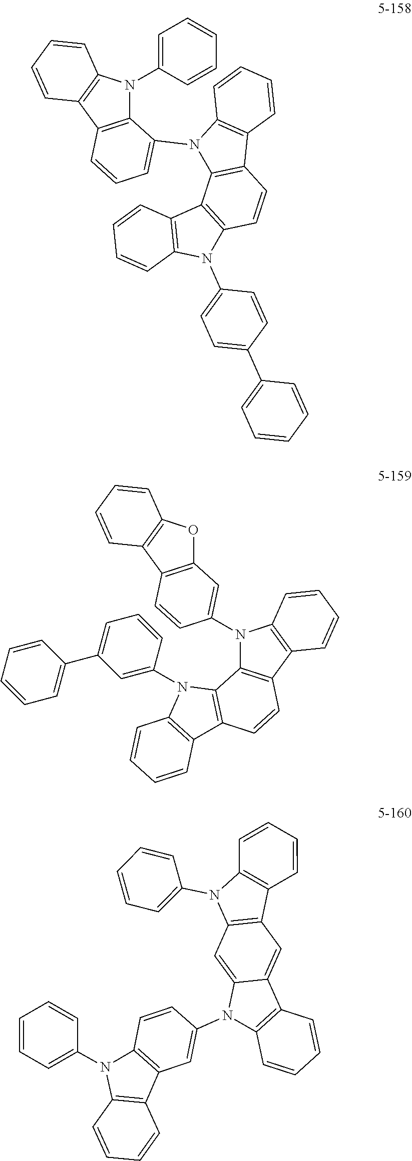

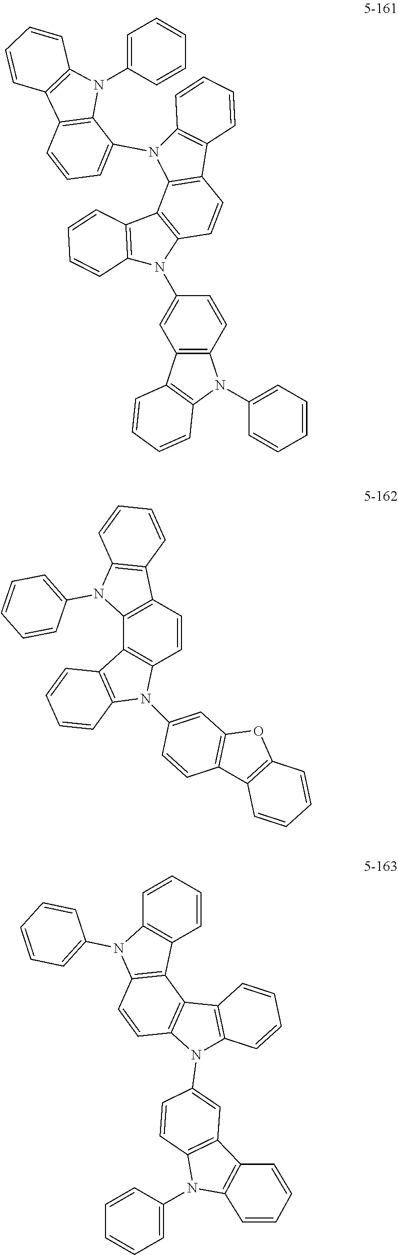

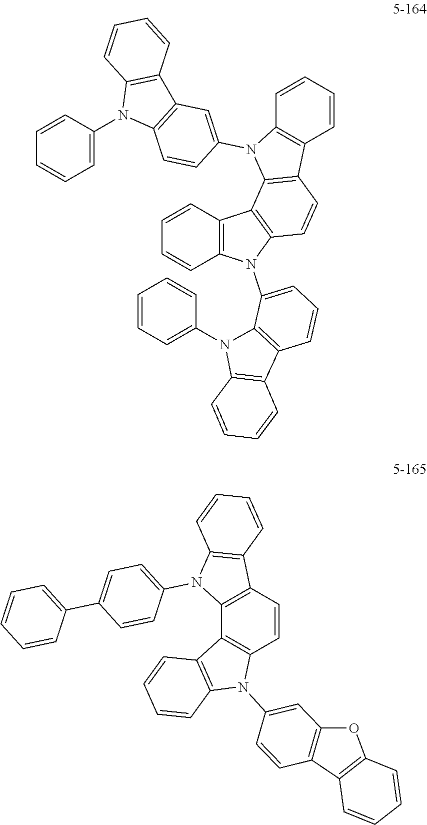

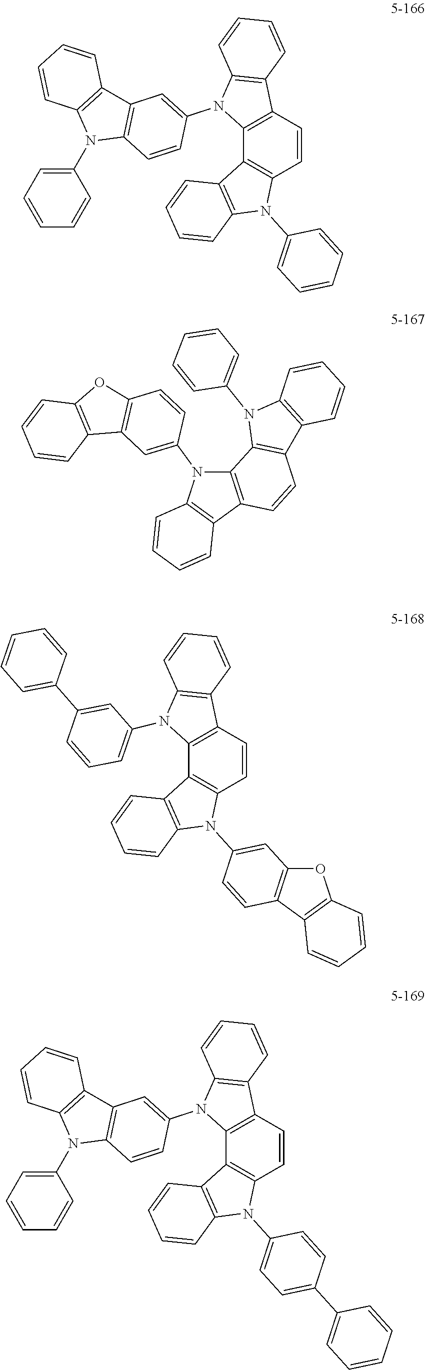

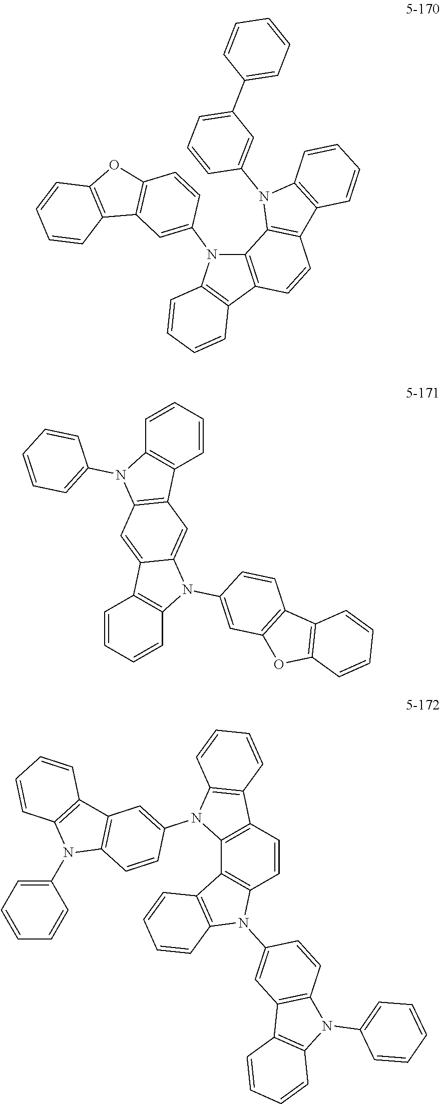

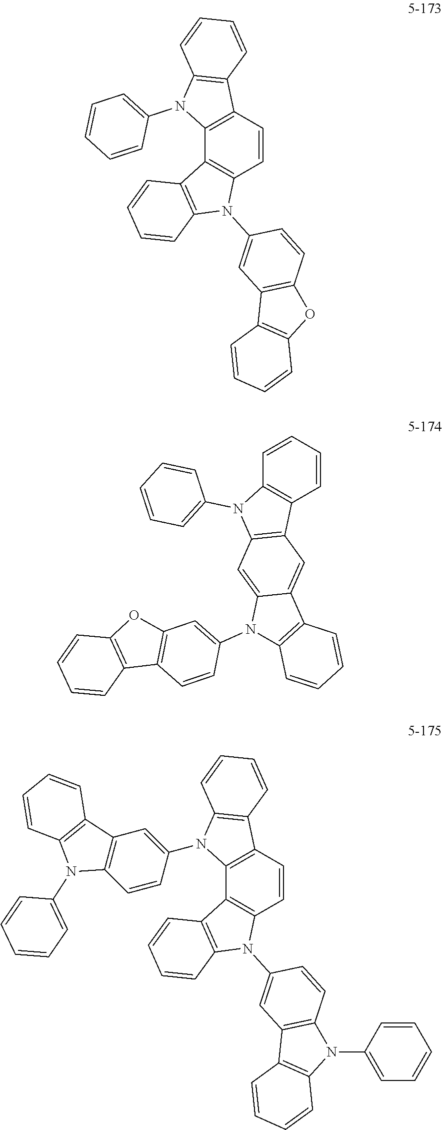

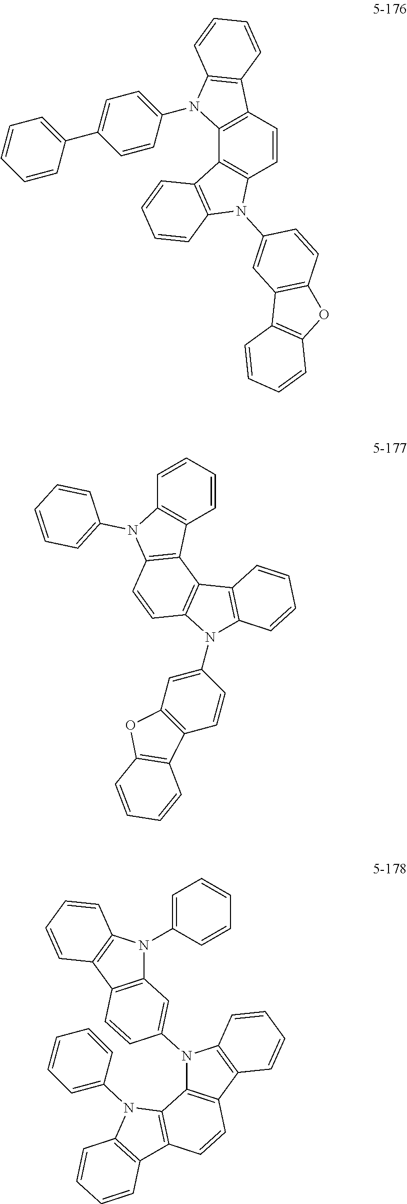

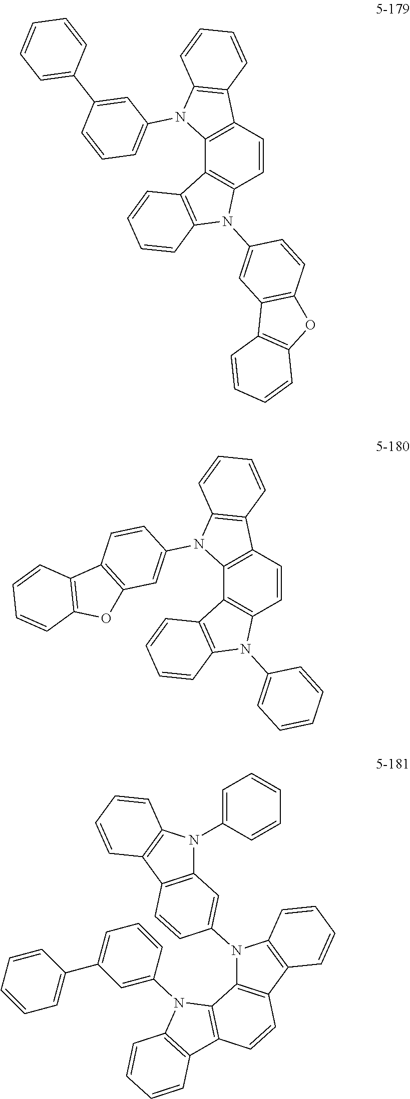

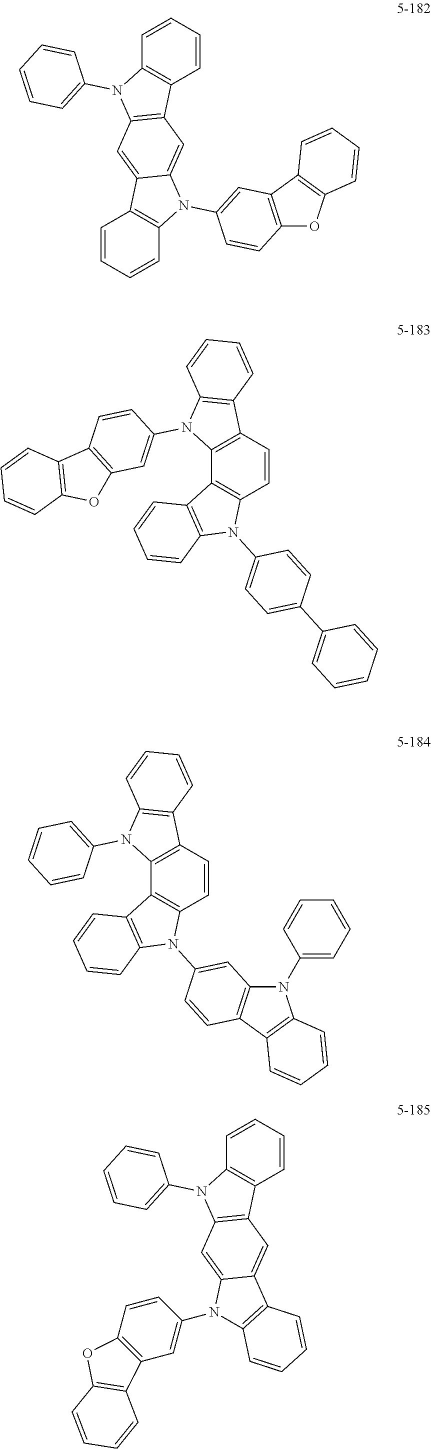

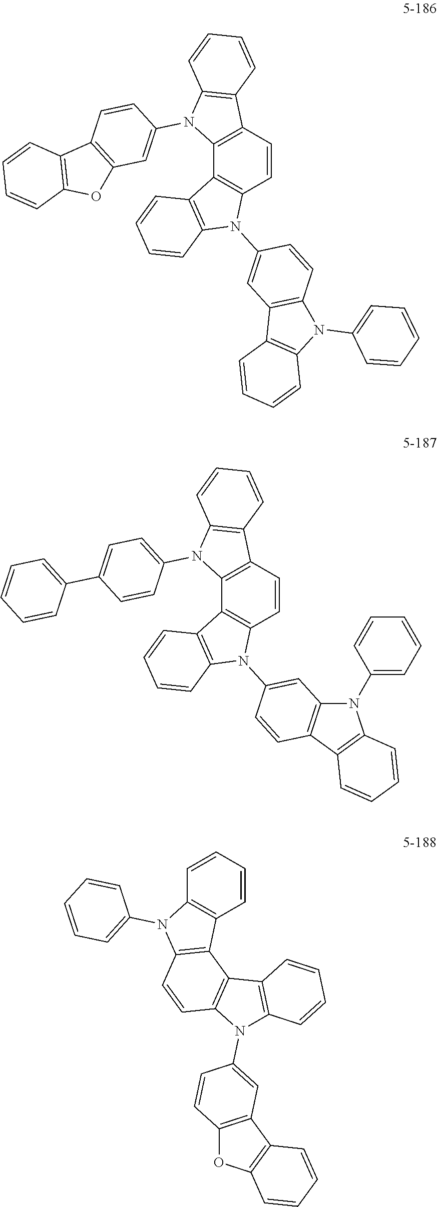

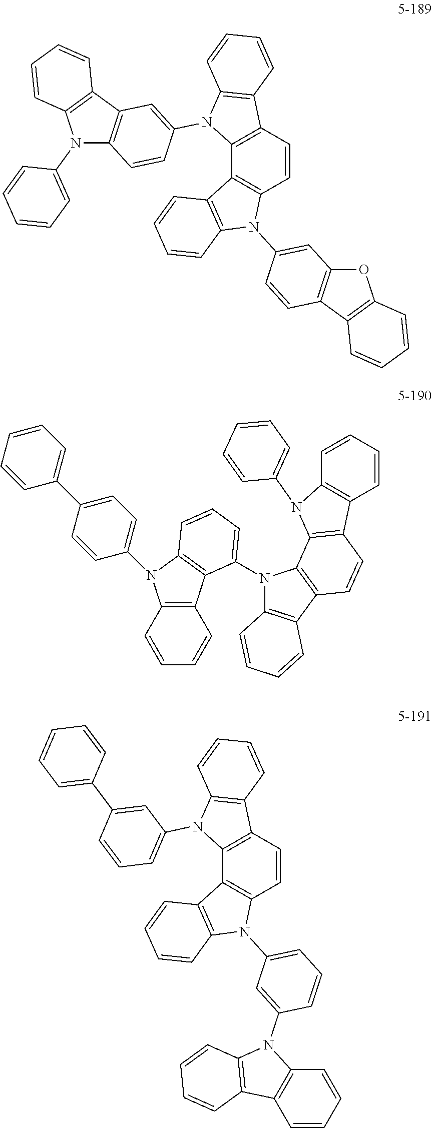

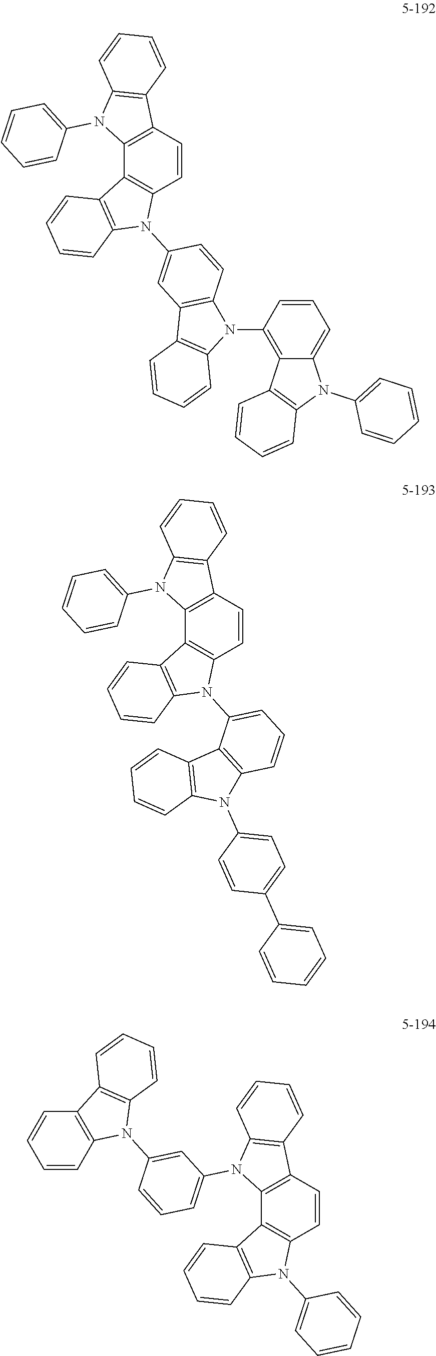

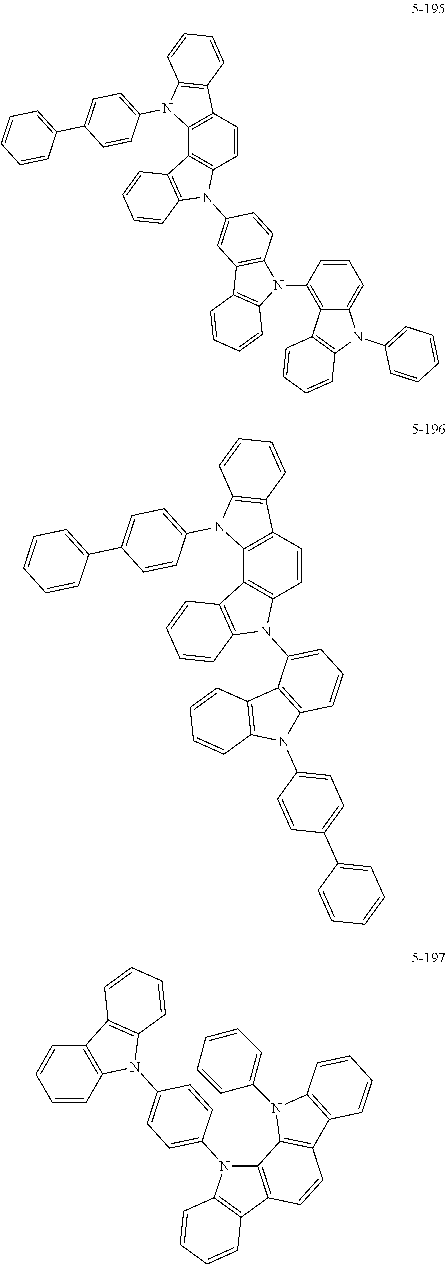

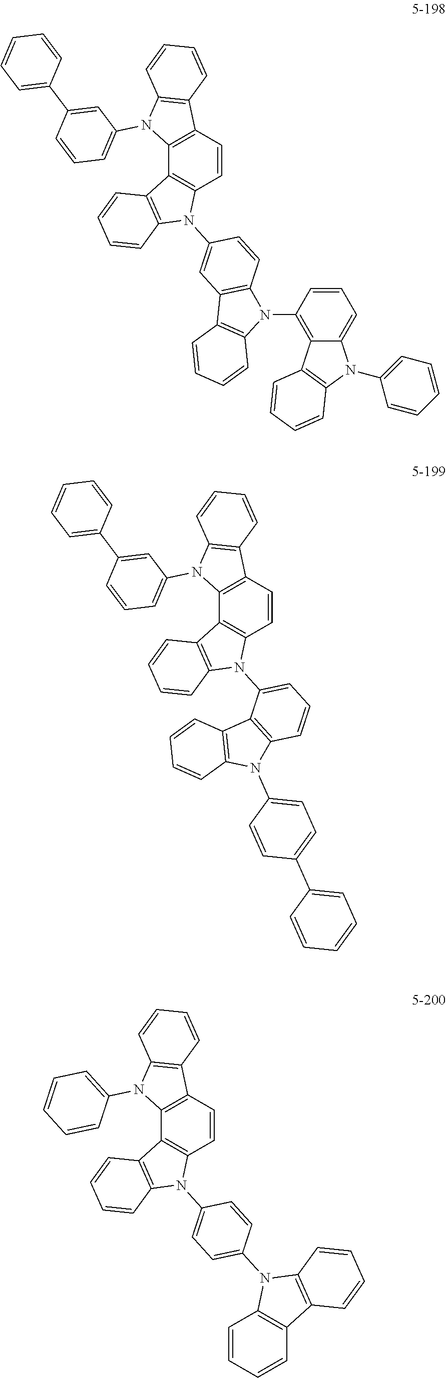

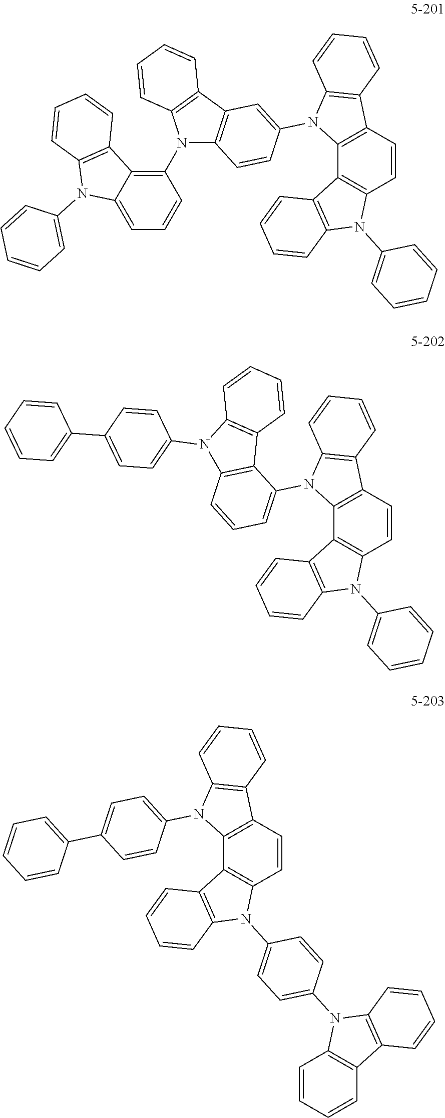

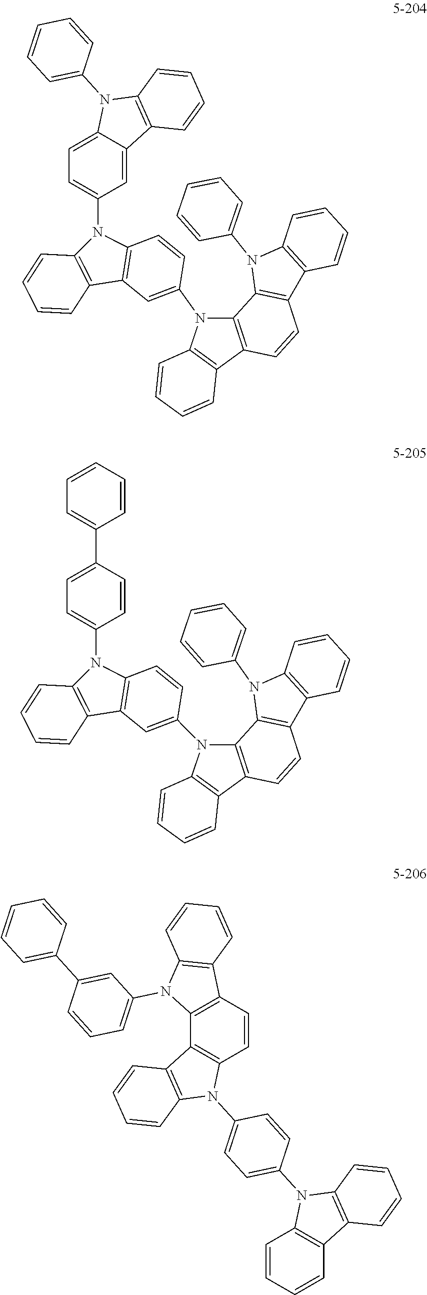

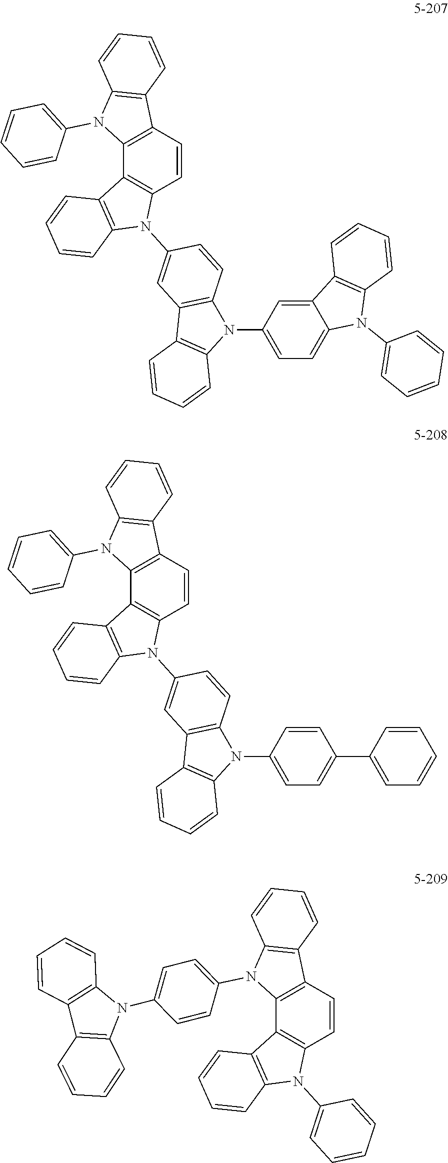

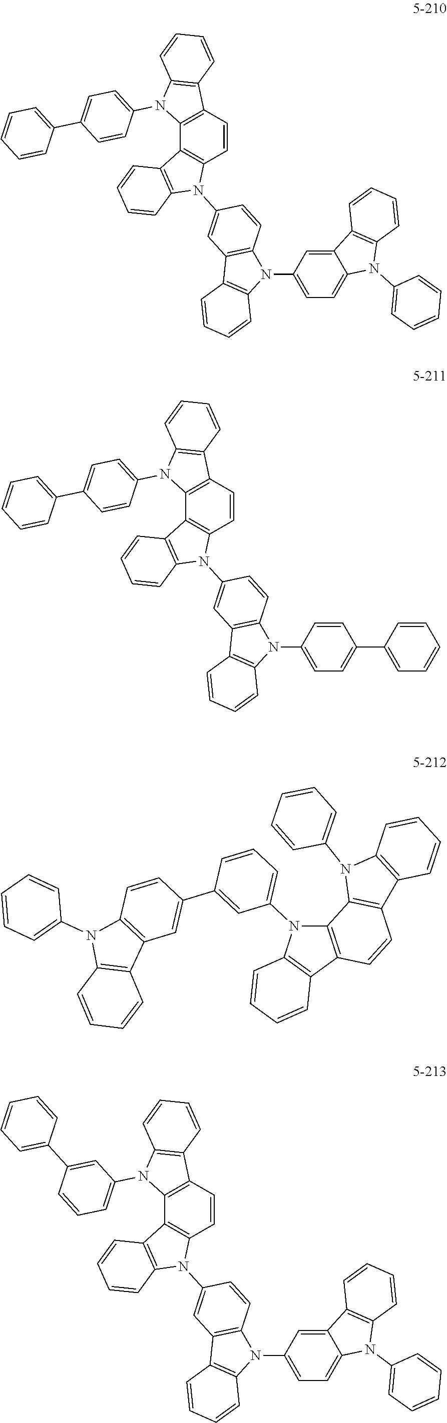

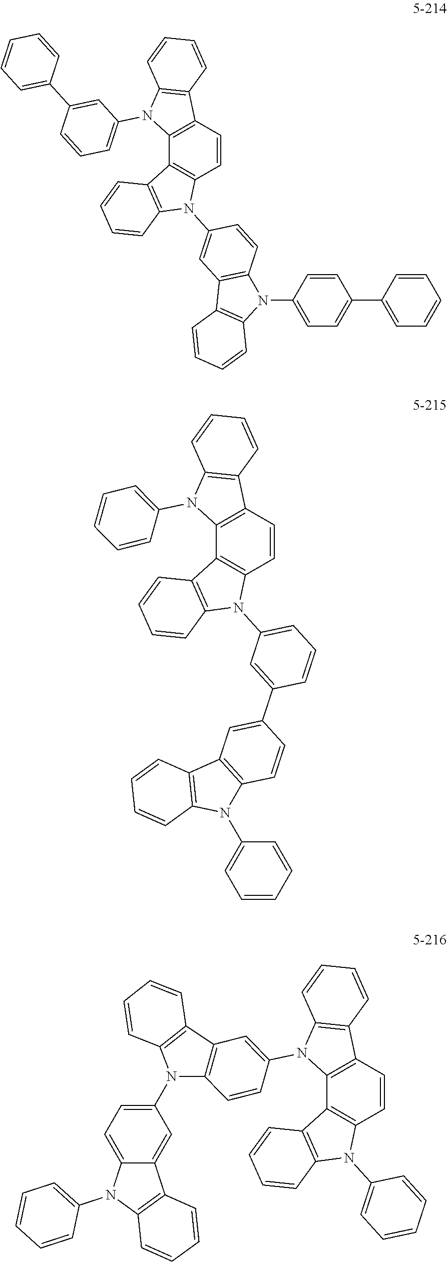

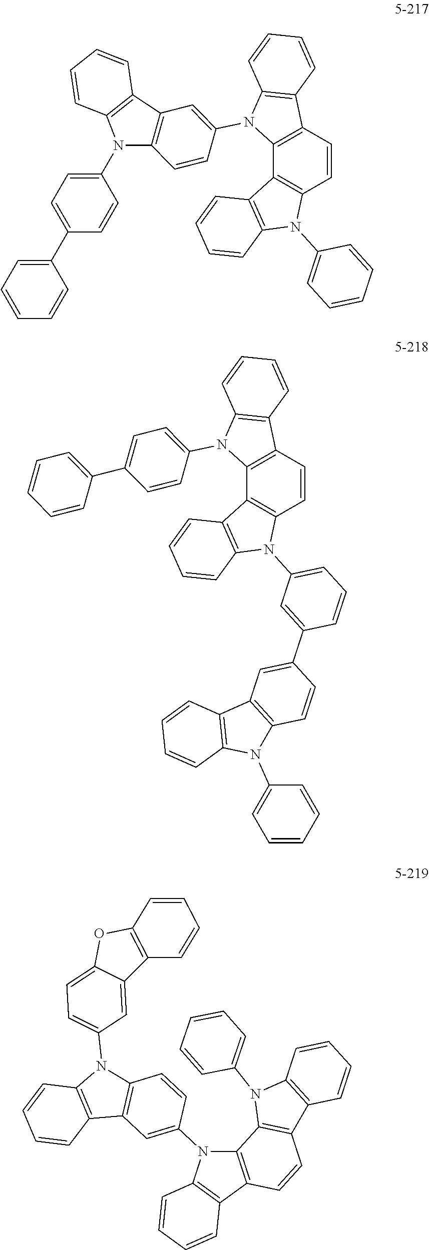

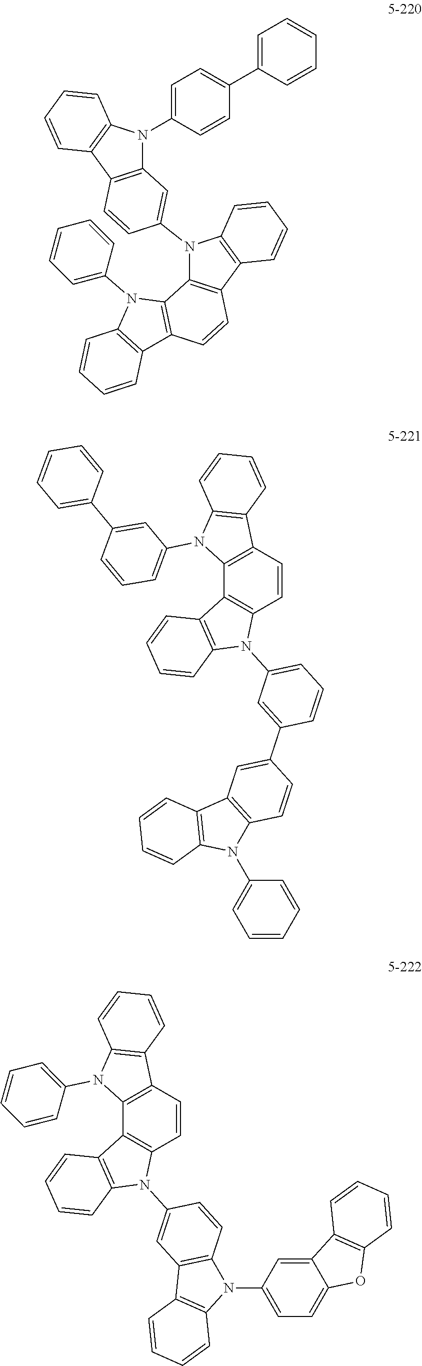

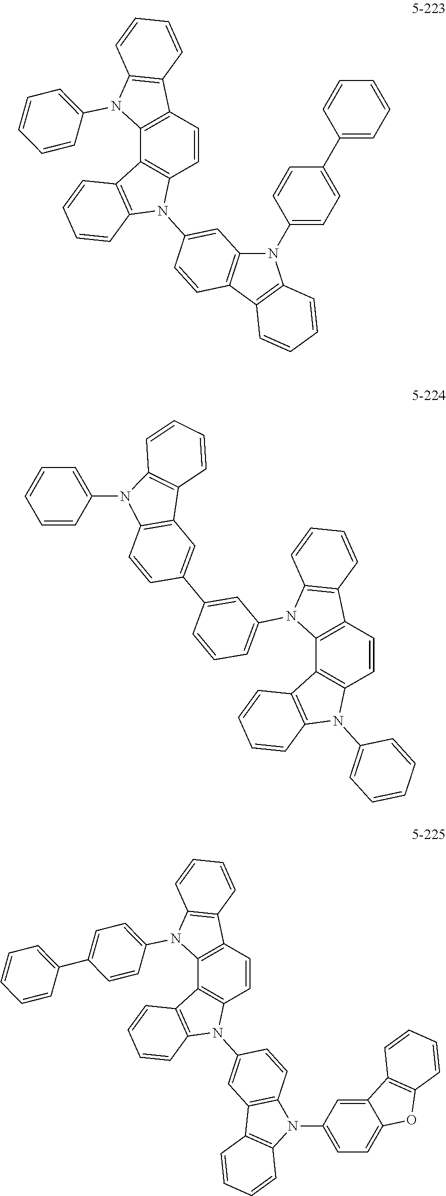

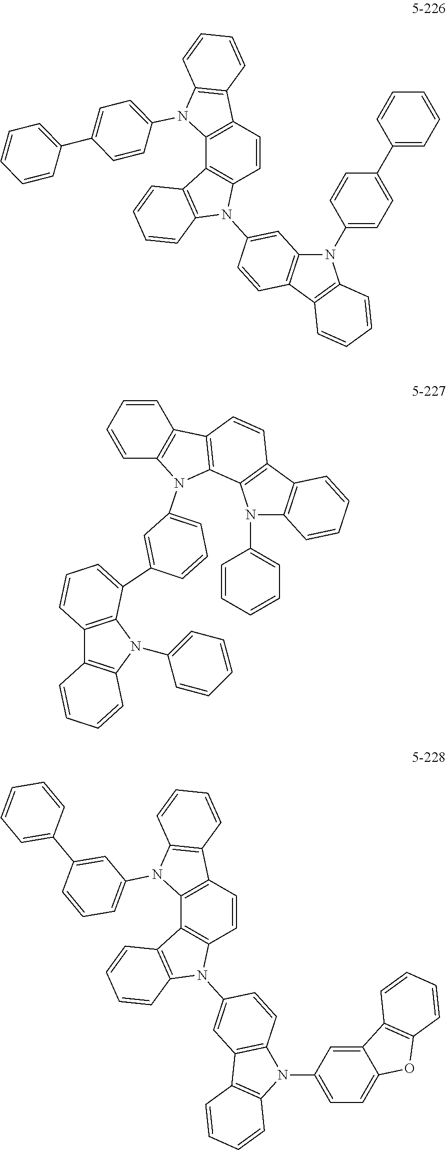

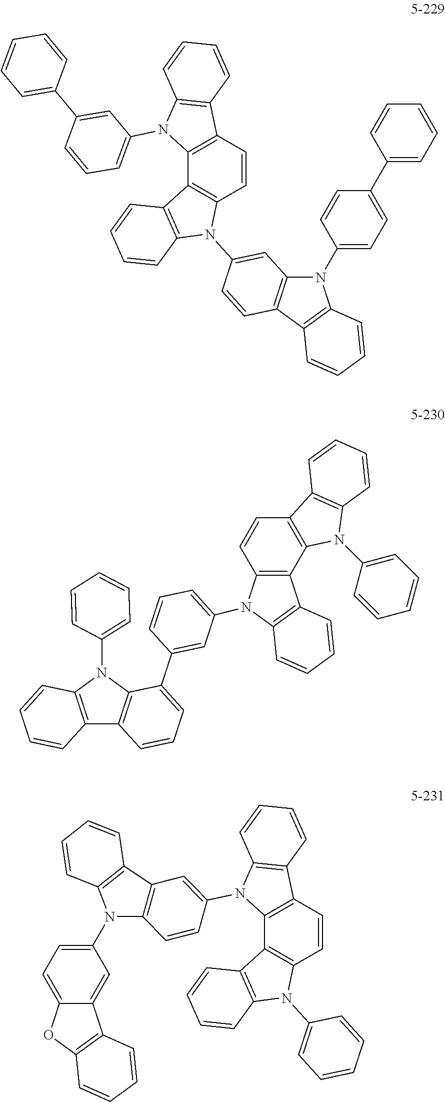

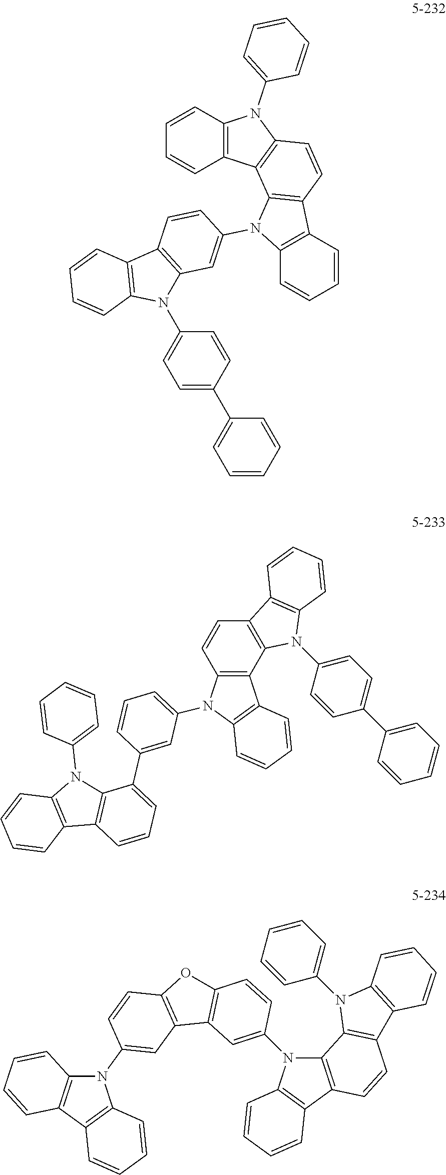

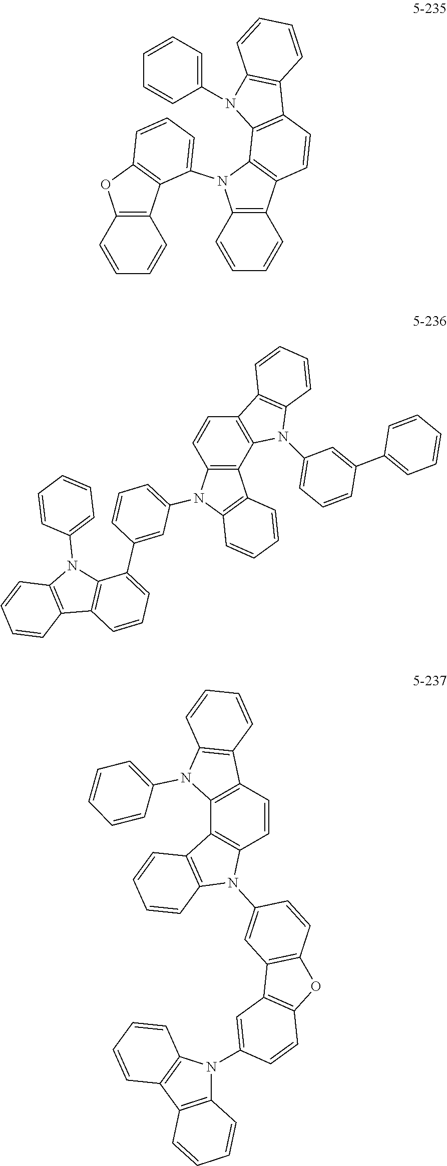

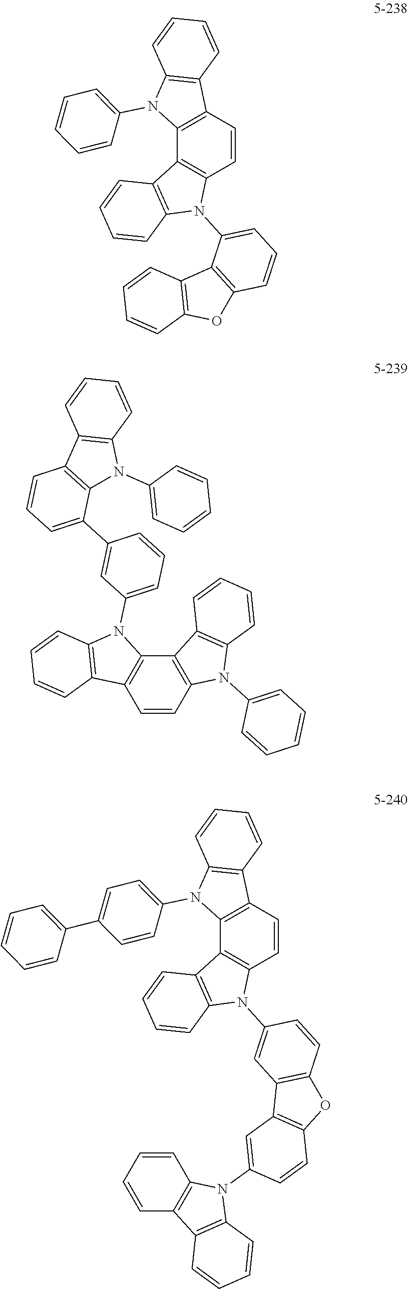

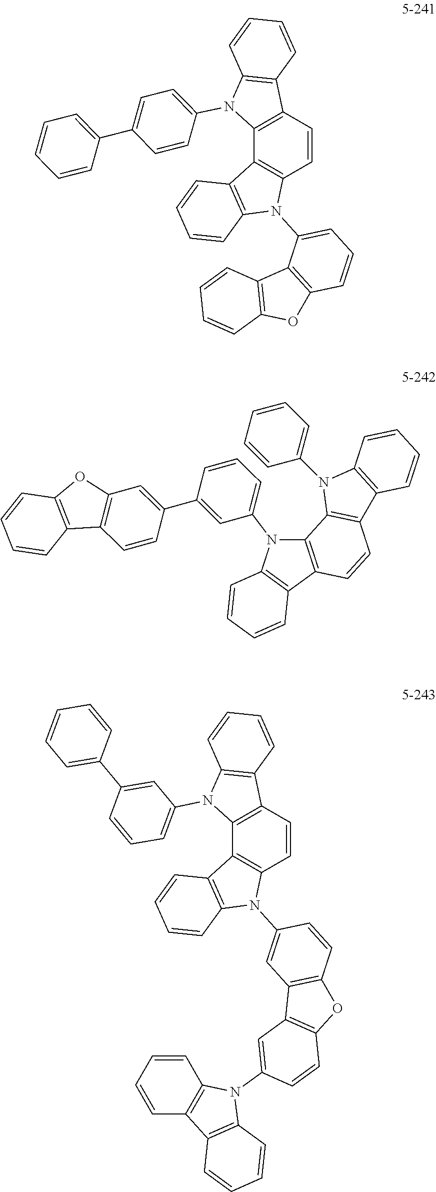

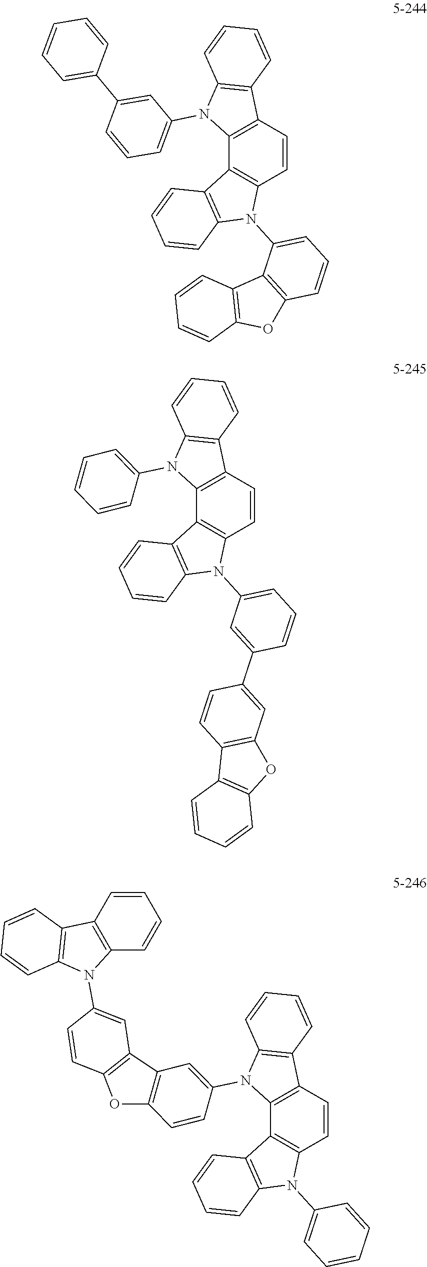

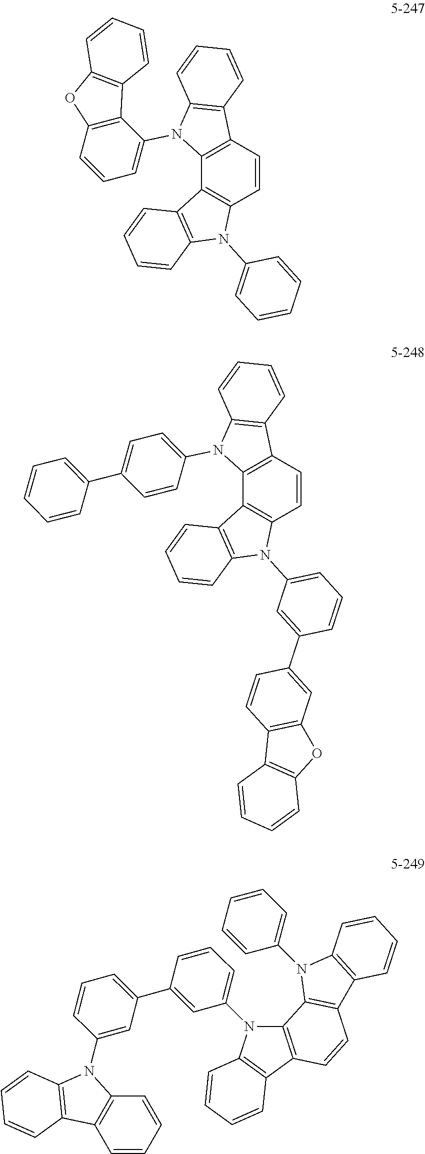

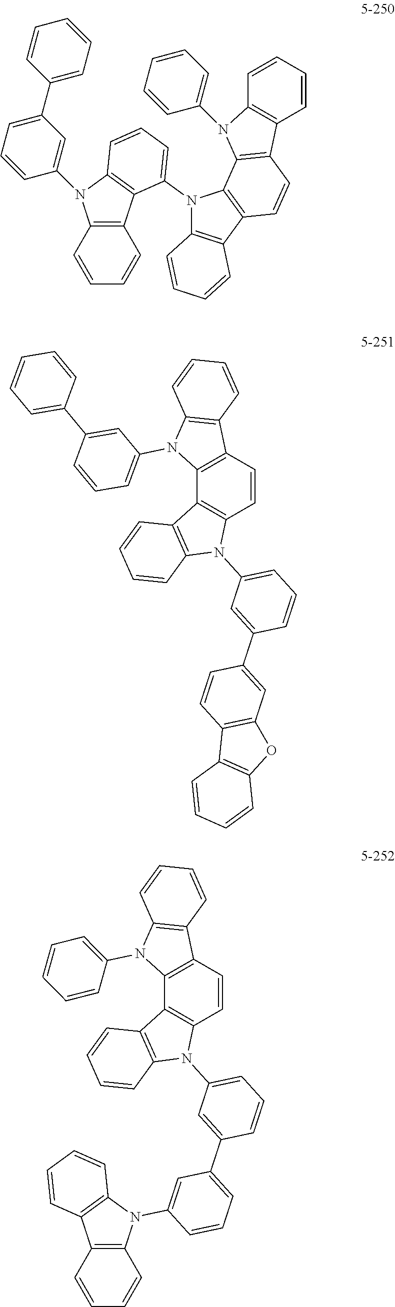

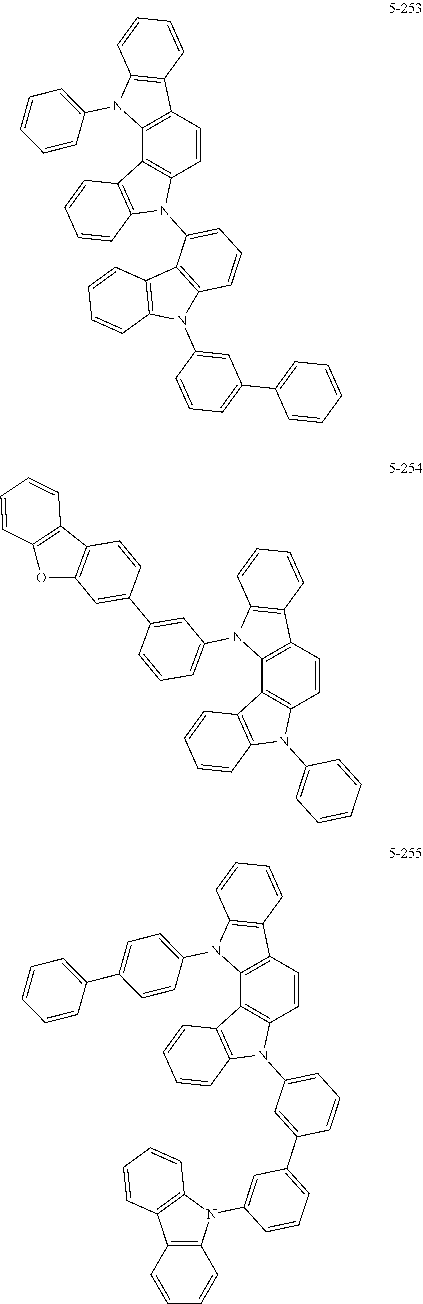

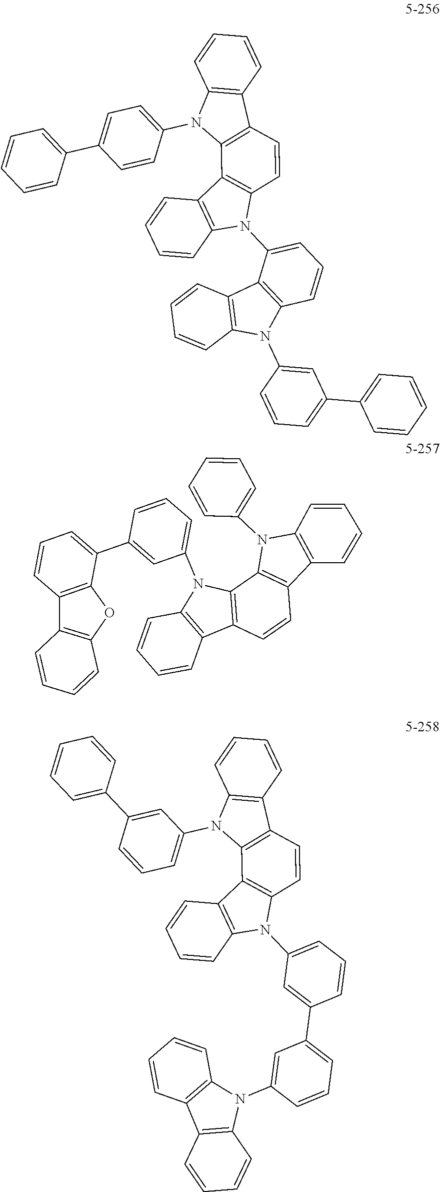

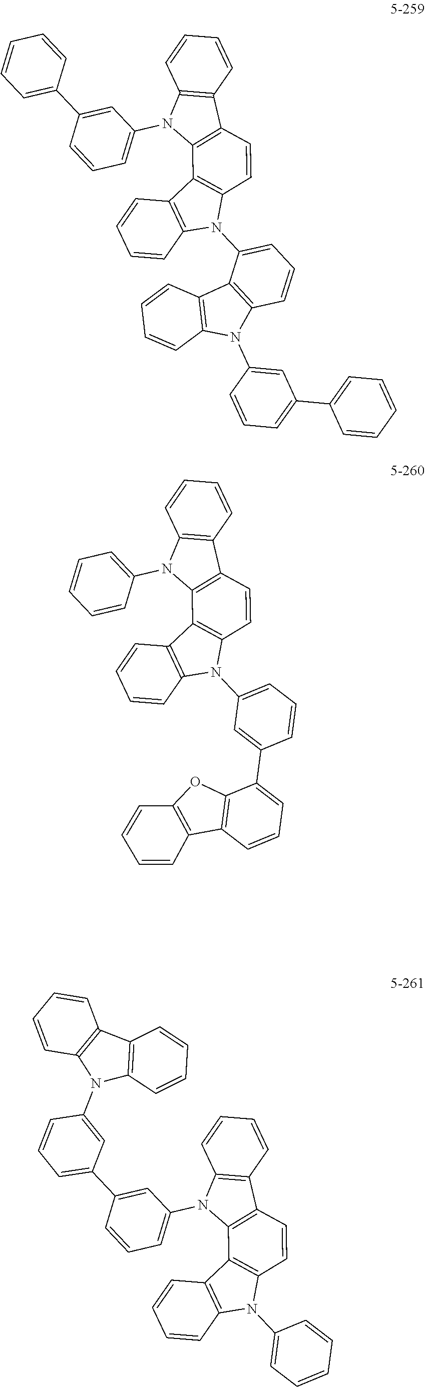

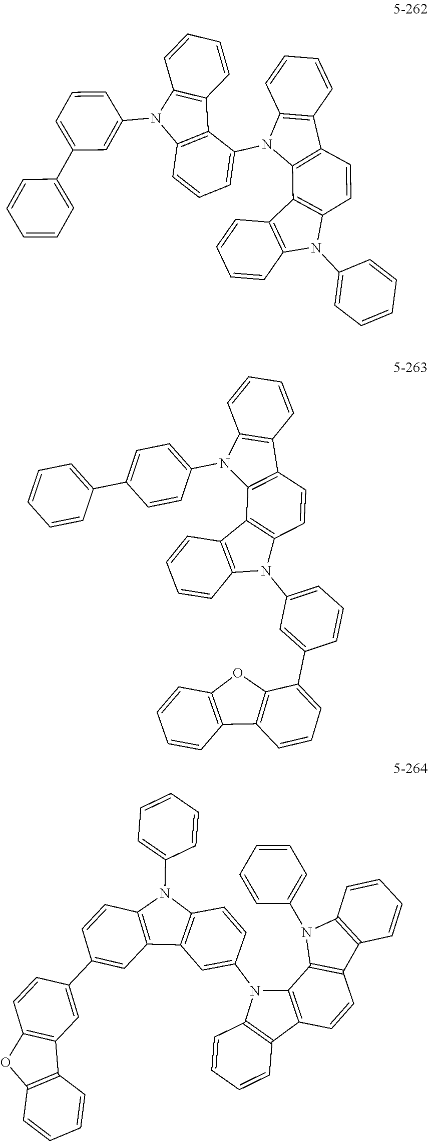

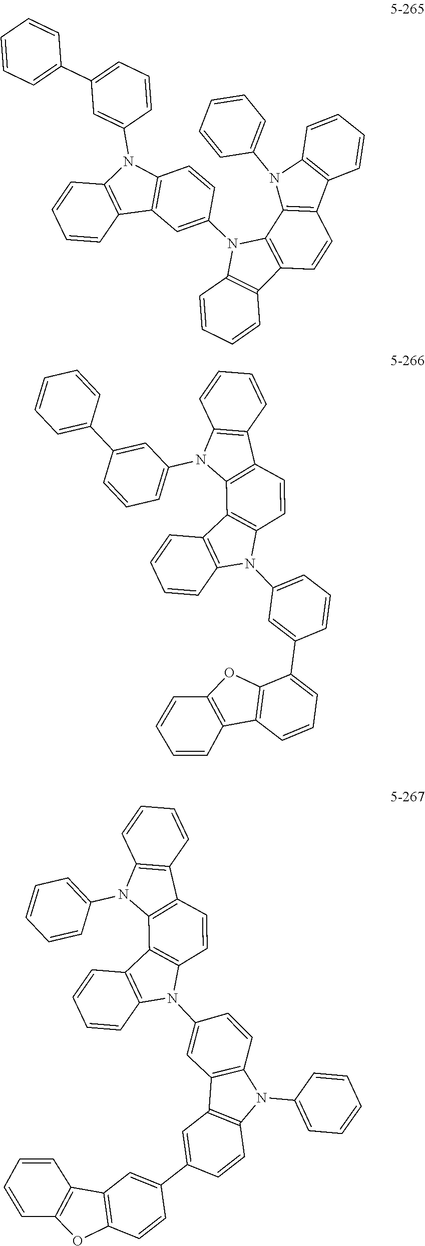

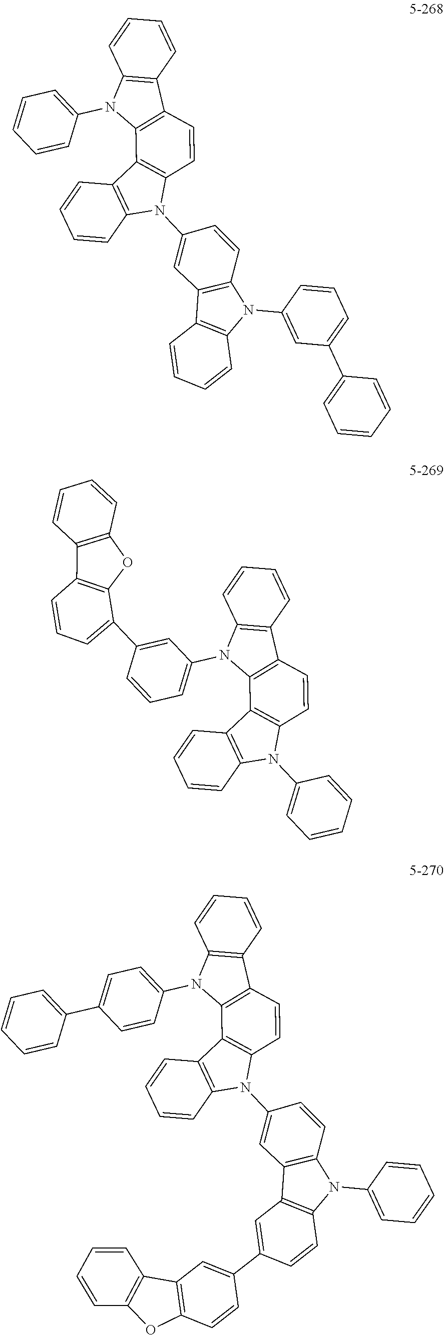

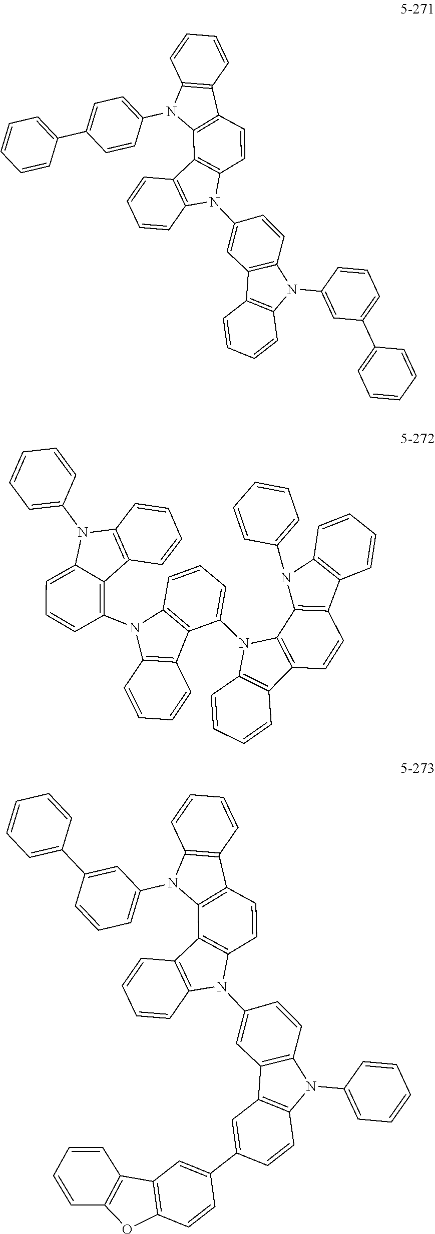

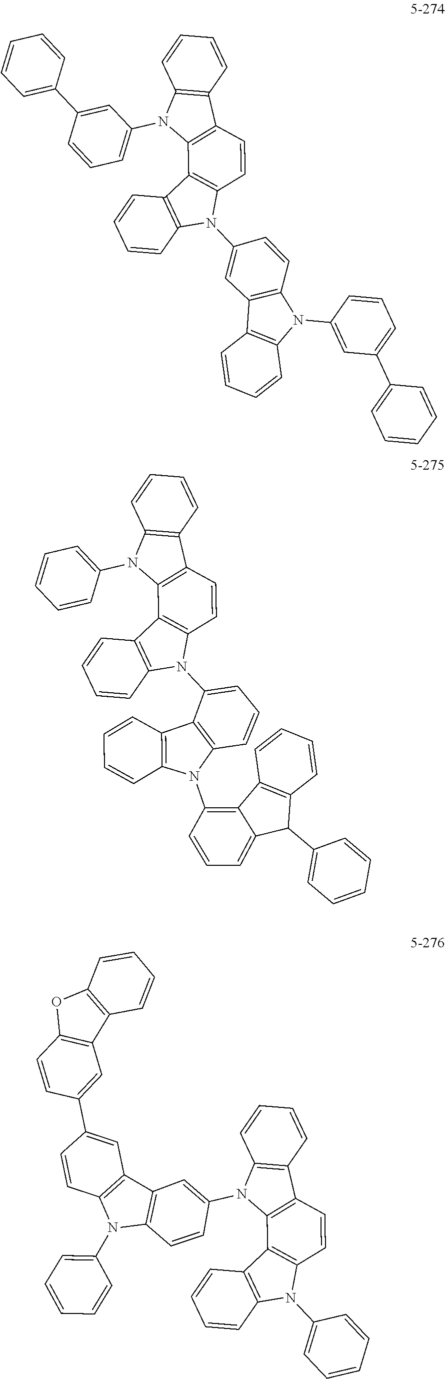

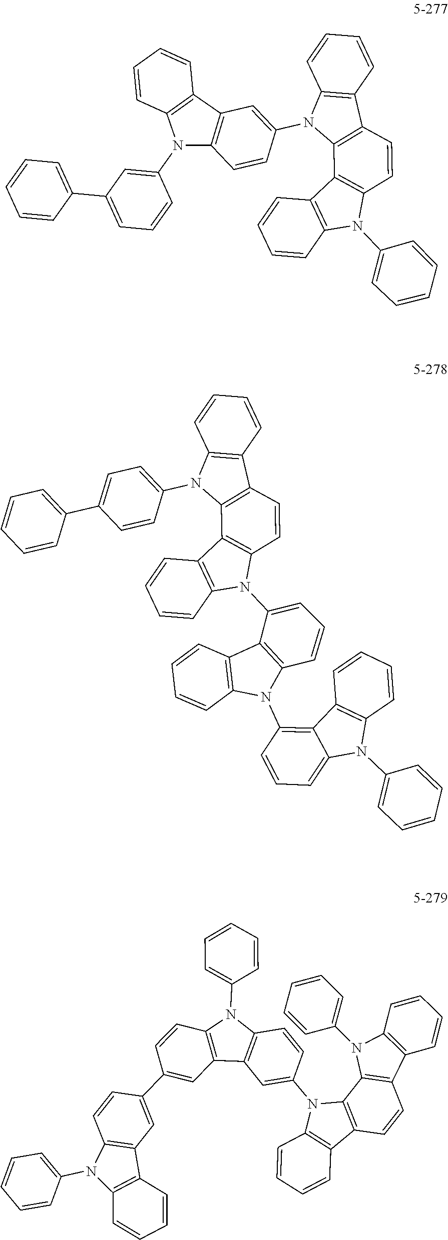

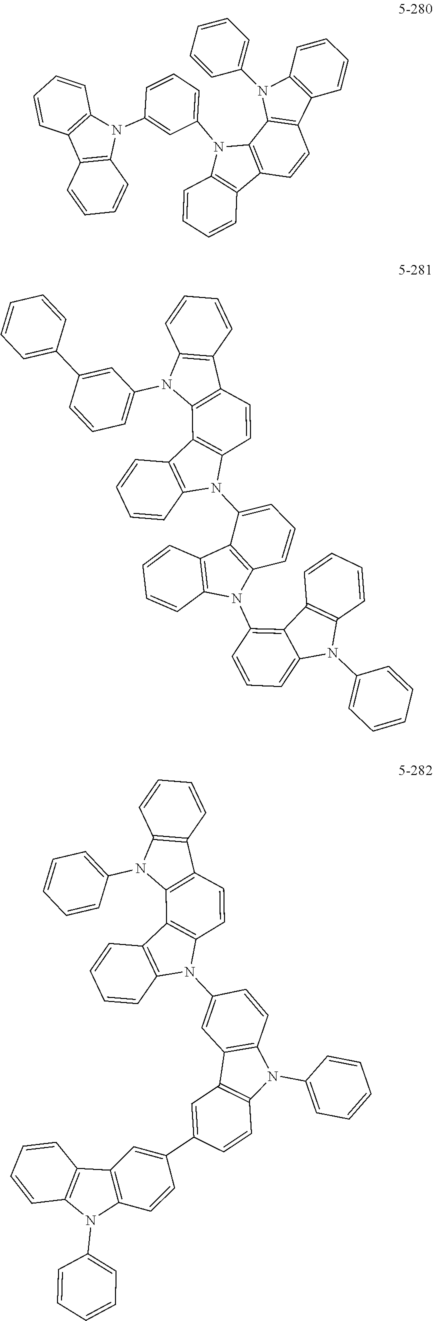

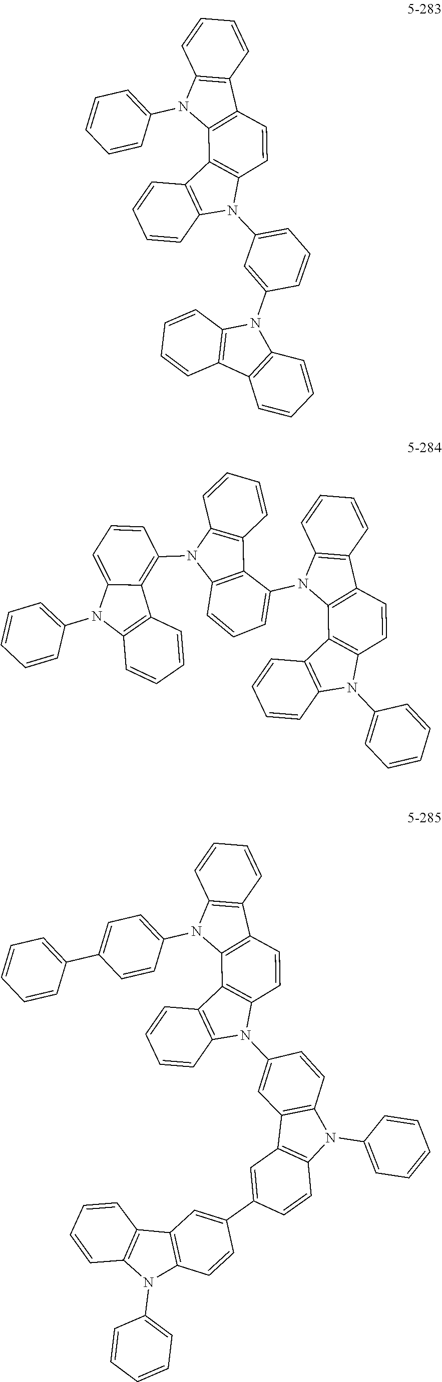

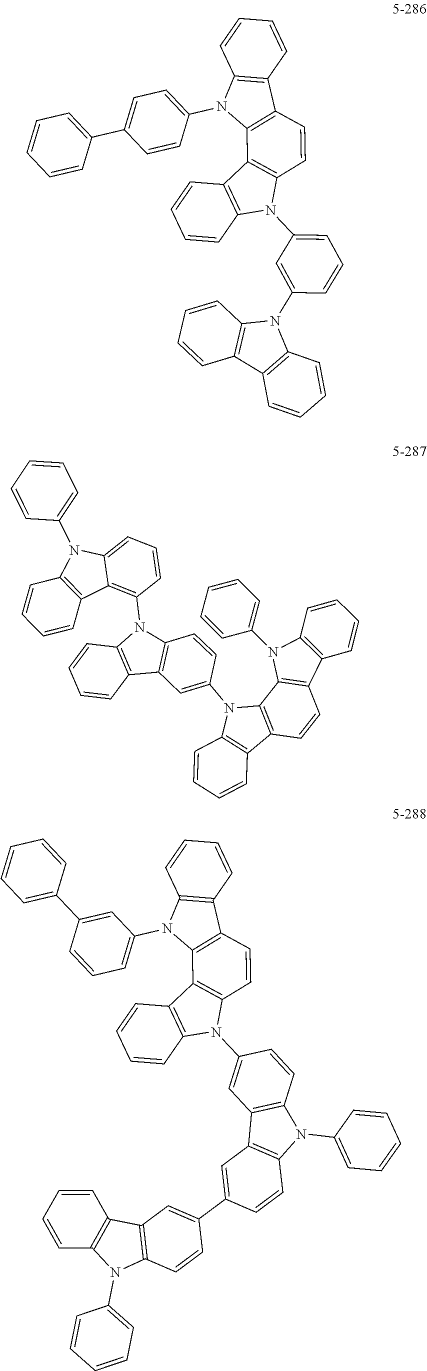

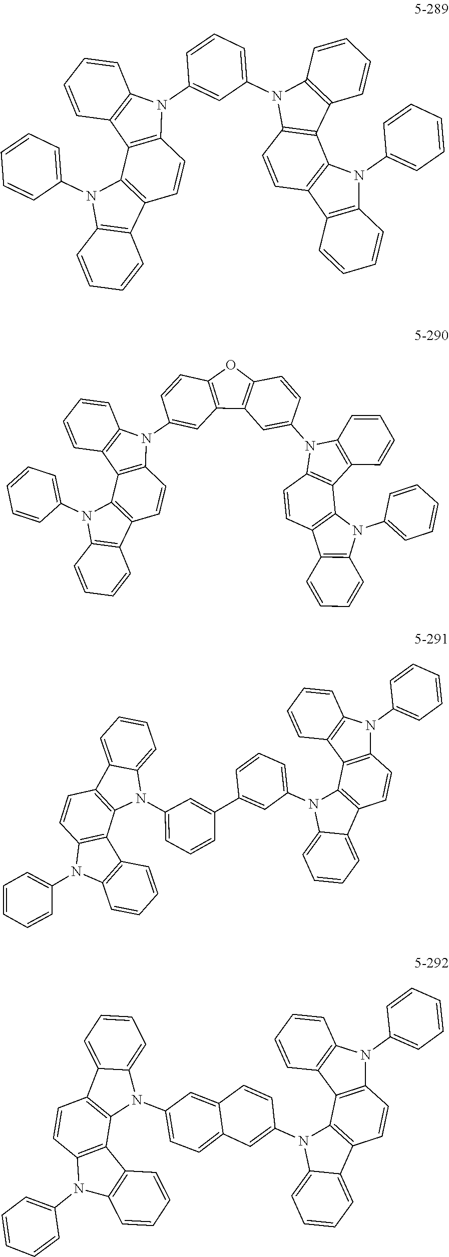

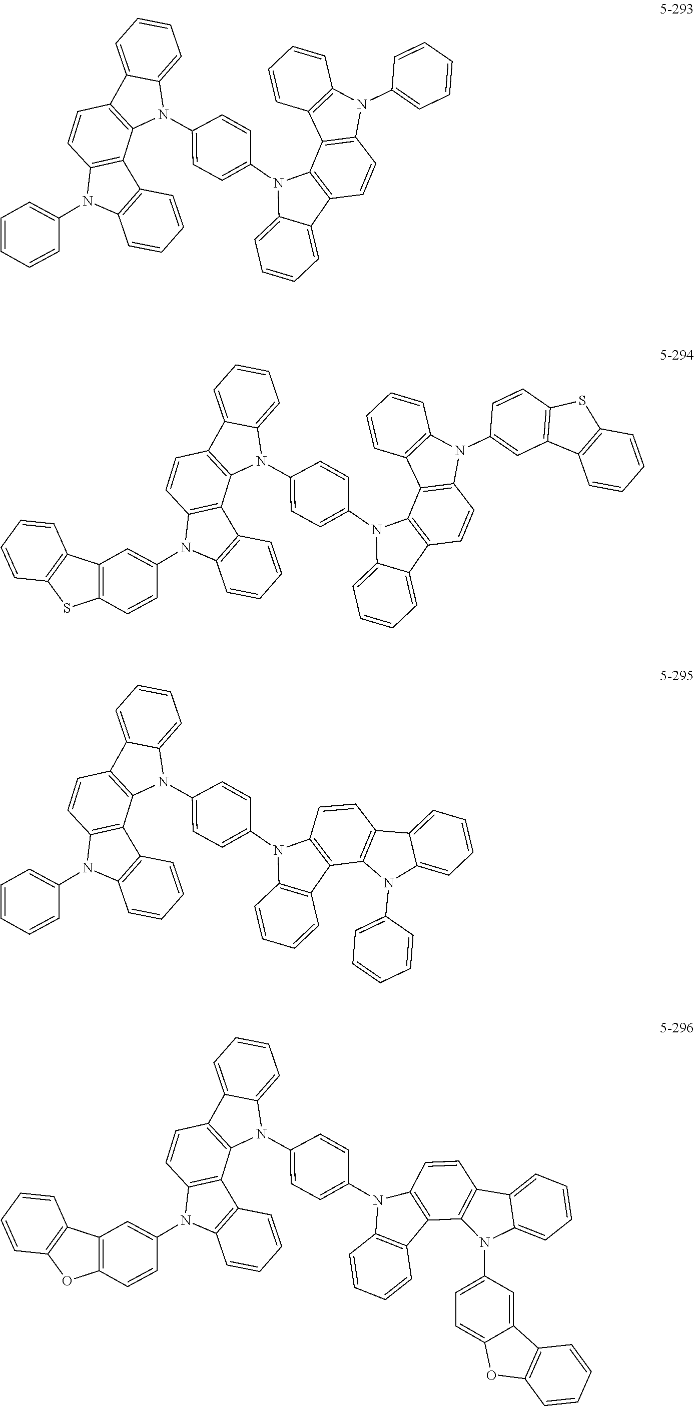

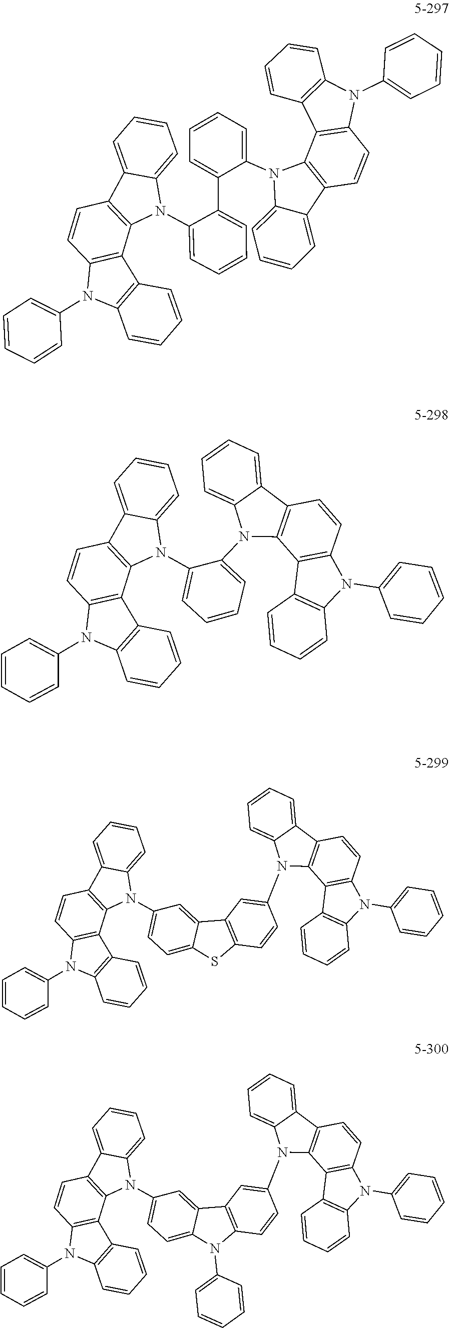

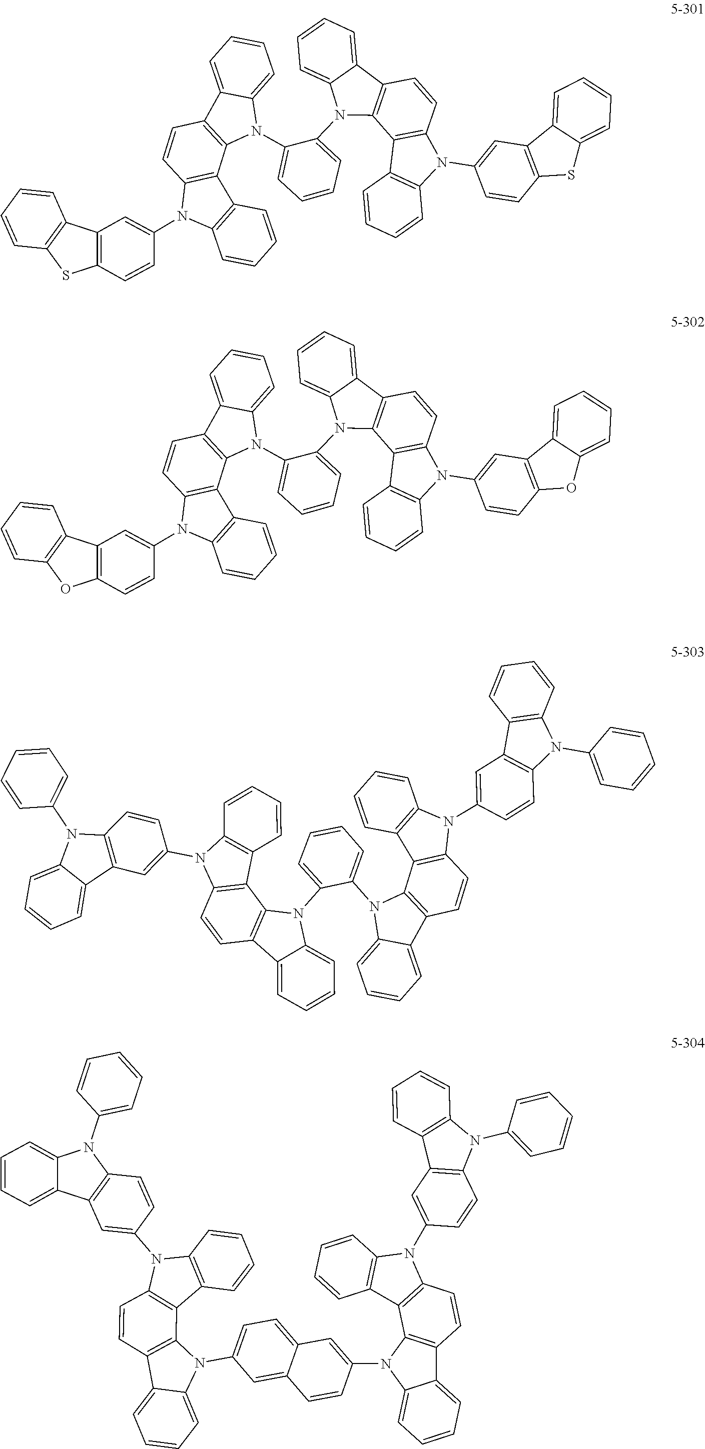

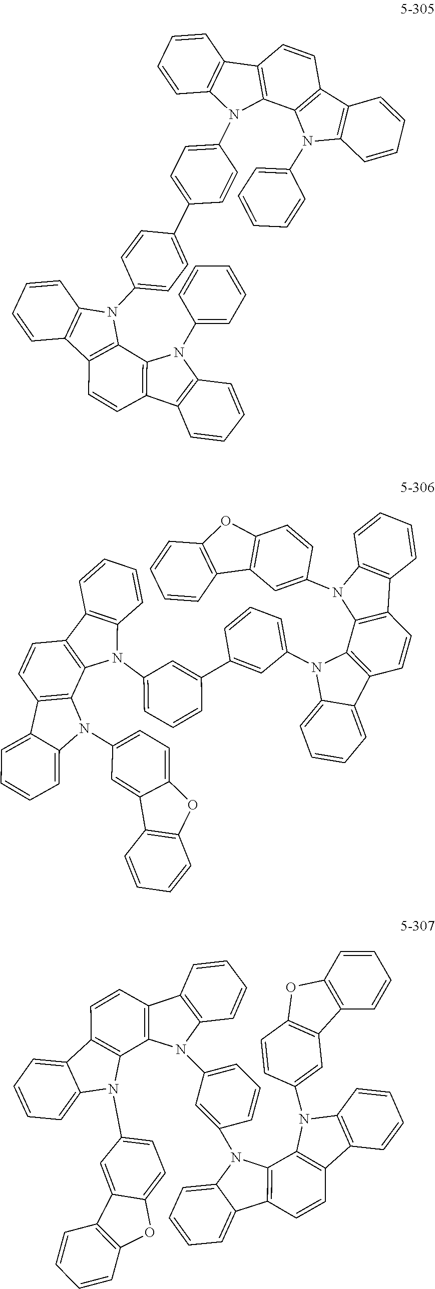

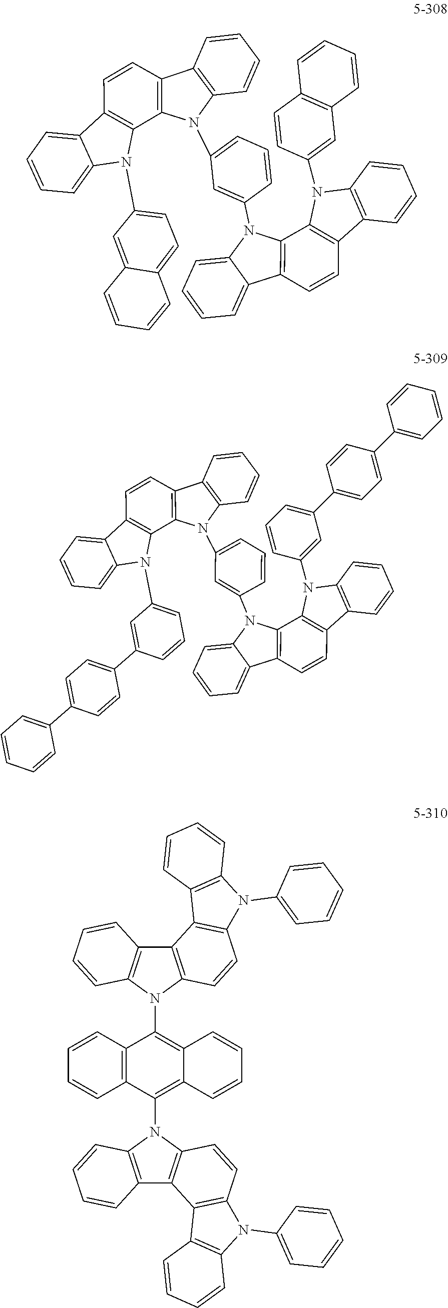

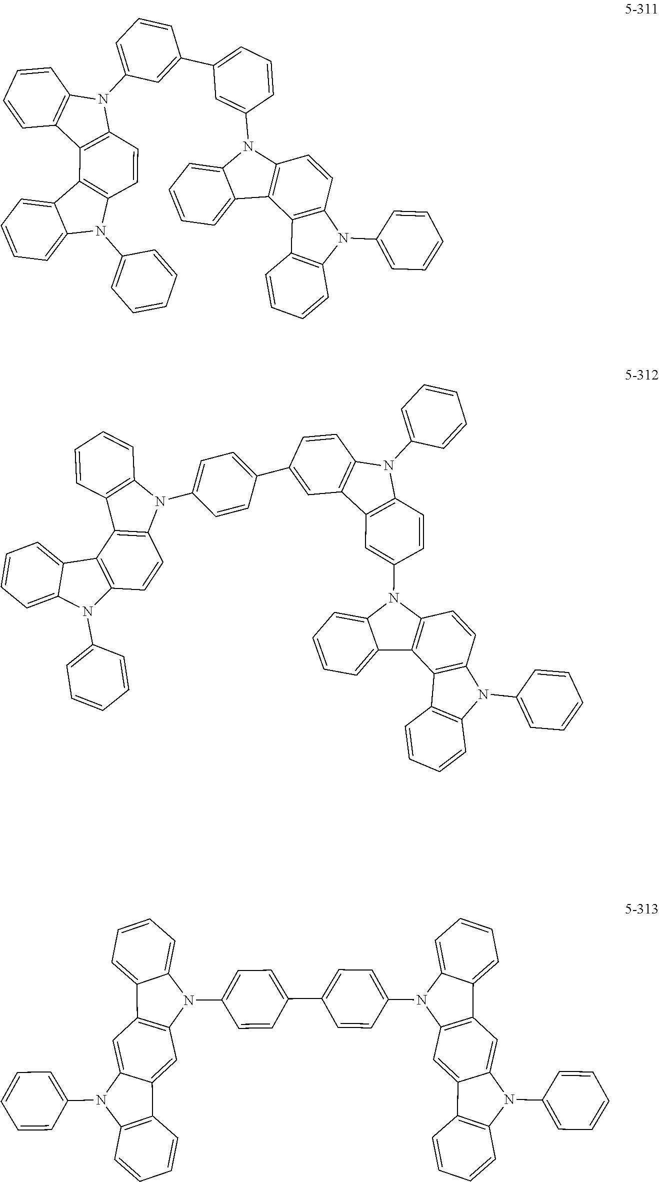

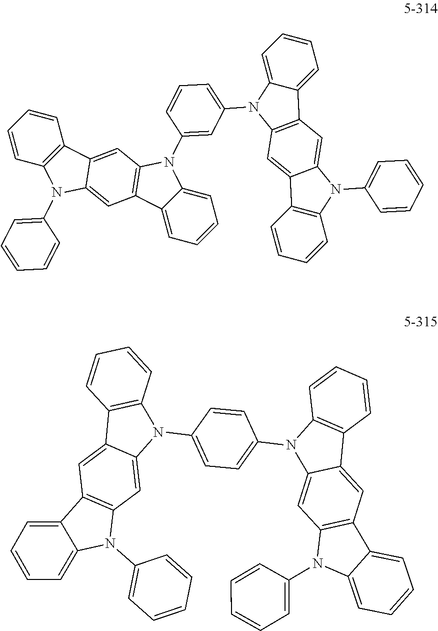

The polycyclic aromatic compounds represented by the general formulae (4a), (4b), (5a), and (5b) above to be used as light emitting dopants in the organic EL device of the present invention preferably have a ΔEST of 0.20 eV or less, which a difference between excited singlet energy (S1) and excited triplet energy (T1). It is more preferably 0.15 eV or less and still more preferably 0.10 eV or less. It is to be noted that the ΔEST (S1-T1) is a value calculated by measuring the light emission spectrum for S1 and the phosphorescence spectrum for T1.

S1 and T1 can also be calculated by theoretical calculation using molecular activation program Gaussian 16. A ΔEST(theo) [S1-T1(theo)] was calculated using values of excited singlet energy [S1(theo)] and excited triplet energy [T1(theo)] obtained by theoretical calculation. In this case, the ΔEST(theo) is preferably 0.60 eV or less and more preferably 0.50 eV or less. A small ΔEST(theo) obtained by the theoretical calculation facilitates reverse intersystem crossing to occur, and can efficiently utilize triplet excitons for light emission, making it possible to expect high light emission efficiency.

Next, a structure of the organic EL device of the present invention will be described with reference to drawings, but the structure of the organic EL device of the present invention is not limited thereto.

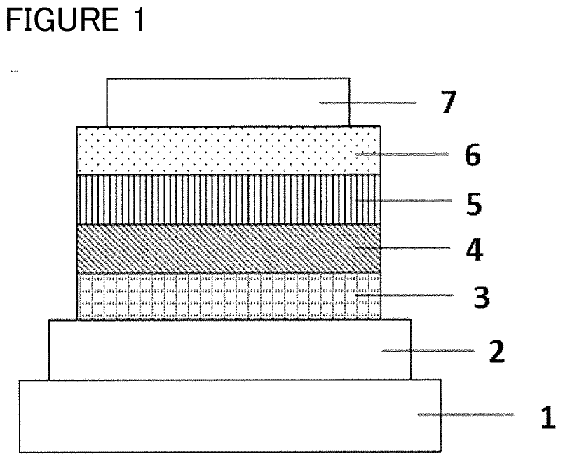

FIG. 1 shows a cross-sectional view of a structure example of a typical organic EL device used in the present invention. Reference numeral 1 denotes a substrate, reference numeral 2 denotes an anode, reference numeral 3 denotes a hole injection layer, reference numeral 4 denotes a hole transport layer, reference numeral 5 denotes a light emitting layer, reference numeral 6 denotes an electron transport layer, and reference numeral 7 denotes a cathode. The organic EL device of the present invention may have an exciton blocking layer adjacent to the light emitting layer, or may have an electron blocking layer between the light emitting layer and the hole injection layer. The exciton blocking layer may be inserted on either the anode side or the cathode side of the light emitting layer or may be inserted on both sides at the same time. The organic EL device of the present invention has the anode, the light emitting layer, and the cathode as essential layers, but preferably has a hole injection/transport layer and an electron injection/transport layer in addition to the essential layers, and further preferably has a hole blocking layer between the light emitting layer and the electron injection/transport layer. The hole injection/transport layer means either or both of the hole injection layer and the hole transport layer, and the electron injection/transport layer means either or both of the electron injection layer and electron transport layer.

It is also possible to have a structure that is the reverse of the structure shown in FIG. 1, that is, the cathode 7, the electron transport layer 6, the light emitting layer 5, the hole transport layer 4, the hole injection layer 3, and the anode 2 can be laminated on the substrate 1, in the order presented. Also, in this case, layers can be added or omitted, as necessary.

### —Substrate—

The organic EL device of the present invention is preferably supported on a substrate. The substrate is not particularly limited and may be a substrate conventionally used for organic EL devices, and for example, a substrate made of glass, transparent plastic, or quartz can be used.

### —Anode—

As the anode material in the organic EL device, a material made of a metal, alloy, or conductive compound having a high work function (4 eV or more), or a mixture thereof is preferably used. Specific examples of such an electrode material include metals such as Au, and conductive transparent materials such as CuI, indium tin oxide (ITO), SnO₂, and ZnO. An amorphous material capable of producing a transparent conductive film such as IDIXO (In₂O₃—ZnO) may also be used. As the anode, these electrode materials may be formed into a thin film by a method such as vapor deposition or sputtering, and then a pattern of a desired form may be formed by photolithography. Alternatively, when a highly precise pattern is not required (about 100 μm or more), a pattern may be formed through a mask of a desired form at the time of vapor deposition or sputtering of the above electrode materials. Alternatively, when a coatable material such as an organic conductive compound is used, a wet film forming method such as a printing method and a coating method can also be used. When light is extracted from the anode, the transmittance is desirably more than 10%, and the sheet resistance as the anode is preferably several hundred Ω/square or less. The film thickness is selected within a range of usually 10 to 1,000 nm, and preferably 10 to 200 nm, although it depends on the material.

### —Cathode—

On the other hand, a material made of a metal (referred to as an electron injection metal), alloy, or conductive compound having a low work function (4 eV or less) or a mixture thereof is used as the cathode material. Specific examples of such an electrode material include sodium, a sodium-potassium alloy, magnesium, lithium, a magnesium/copper mixture, a magnesium/silver mixture, a magnesium/aluminum mixture, a magnesium/indium mixture, an aluminum/aluminum oxide (Al₂O₃) mixture, indium, a lithium/aluminum mixture, and a rare earth metal. Among them, in terms of electron injection properties and durability against oxidation and the like, a mixture of an electron injection metal with a second metal that has a higher work function value than the electron injection metal and is stable, for example, a magnesium/silver mixture, a magnesium/aluminum mixture, a magnesium/indium mixture, an aluminum/aluminum oxide (Al₂O₃) mixture, a lithium/aluminum mixture, or aluminum is suitable. The cathode can be produced by forming a thin film from these cathode materials by a method such as vapor deposition and sputtering. The sheet resistance as the cathode is preferably several hundred Ω/square or less, and the film thickness is selected within a range of usually 10 nm to 5 μm, and preferably 50 to 200 nm. To transmit the light emitted, either one of the anode and the cathode of the organic EL device is favorably transparent or translucent because light emission brightness is improved.

The above metal is formed to have a film thickness of 1 to 20 nm on the cathode, and then a conductive transparent material mentioned in the description of the anode is formed on the metal, so that a transparent or translucent cathode can be produced. By applying this process, a device in which both anode and cathode have transmittance can be produced.

### —Light Emitting Layer—

The light emitting layer is a layer that emits light after holes and electrons respectively injected from the anode and the cathode are recombined to form excitons, and the light emitting layer includes the light emitting dopant and the hosts. The mixing ratio of the light emitting dopant and the host is preferably adjusted and used so that the ratio of the light emitting dopant is 0.10 to 10% and that of the host is 99.9 to 90%, and it is more preferable that the ratio of the light emitting dopant is 1.0 to 5.0% and that of the host is 99 to 95%, and it is still more preferable that the ratio of the light emitting dopant is 1.0 to 3.0% and that of the host is 99 to 97%. In the present description, % denotes % by mass unless otherwise noted.

The compound of the present invention represented by the general formula (1) above can be used as the host in the light emitting layer.

When the compound of the present invention represented by the general formula (1) above is included as the first host material, it is preferable to use the compound represented by the general formula (6) above as the second host. It is also preferable that the compound represented by the general formula (1) above is an electron transporting host, and the compound represented by the general formula (6) above is a hole transporting host. Here, the mixing ratio of the first host and the second host is preferably adjusted and used so that the ratio of the first host is 10 to 90% and that of the second host is 90 to 10%. More preferably, the amount of the first host is 30 to 70% and the amount of the second host is 70 to 30%. Still more preferably, the amount of the first host is 30 to 50% and the amount of the second host is 70 to 50%.

In the light emitting layer, the host of the present invention represented by the general formula (1) above or general formula (6) above may be used singly, or two or more different compounds thereof may be used. One or more known hosts may be used in combination, and the amount thereof used may be 50% or less and preferably 25% or less based on the total amount of the host materials.

The other known host that can be used is a compound having the ability to transport hole, the ability to transport electron, and a high glass transition temperature, and preferably has a higher T1 than the T1 of the light emitting dopant. Specifically, the host has a higher T1 than the T1 of the light emitting dopant preferably by 0.010 eV or more, more preferably by 0.030 eV or more, and still more preferably by 0.10 eV or more. A TADF-active compound may also be used as the host material, and this compound preferably has a ΔEST of 0.20 eV or less.

The other known hosts are known in a large number of patent literatures and the like, and hence may be selected from them. Specific examples of the host include, but are not particularly limited to, various metal complexes typified by metal complexes of indole derivatives, carbazole derivatives, indolocarbazole derivatives, triazole derivatives, oxazole derivatives, oxadiazole derivatives, imidazole derivatives, phenylenediamine derivatives, arylamine derivatives, styrylanthracene derivatives, fluorenone derivatives, stilbene derivatives, triphenylene derivatives, carborane derivatives, porphyrin derivatives, phthalocyanine derivatives, and 8-quinolinol derivatives, and metal phthalocyanine, and metal complexes of benzoxazole and benzothiazole derivatives; and polymer compounds such as poly(N-vinyl carbazole)derivatives, aniline-based copolymers, thiophene oligomers, polythiophene derivatives, polyphenylene derivatives, polyphenylene vinylene derivatives, and polyfluorene derivatives.

When a plurality of hosts is used, each host is deposited from different deposition sources, or a plurality of hosts is premixed before vapor deposition to form a premix, whereby a plurality of hosts can be simultaneously deposited from one deposition source.

As the method of premixing, a method by which hosts can be mixed as uniformly as possible is desirable, and examples thereof include, but are not limited to, milling, a method of heating and melting hosts under reduced pressure or under an inert gas atmosphere such as nitrogen, and sublimation. The premixed mixture may be in the form of powder, a stick, or a granule.

When the compound of the present invention represented by the general formula (1) is used as a host, the energy level of the highest occupied molecular orbital (HOMO) obtained by structure optimization calculation using density functional calculation B3LYP/6-31G(D) is preferably −4.7 eV or less and more preferably in the range of −5.9 eV to −4.7 eV.

Also the energy level of the lowest unoccupied molecular orbital (LUMO) obtained by the above structure optimization calculation is preferably −2.5 eV or more and more preferably in the range of −1.8 eV to −1.2 eV.

When the compound of the present invention represented by the general formula (1) is used as the host, the difference (absolute value) between the HOMO energy level and the LUMO energy level is preferably in the range of 2.5 to 5.0 eV and more preferably in the range of 3.0 to 4.5 eV.

It is preferable to use the light emitting dopant in the light emitting layer, which is the polycyclic aromatic compound material represented by the general formula (4a), (4b), (5a), or (5b) above.

The light emitting layer can contain two or more light emitting dopants. For example, the compound represented by the above-described general formula (1), or the polycyclic aromatic compound material represented by the general formula (4a), (4b), (5a) or (5b), can be used in combination of two or more thereof, or a light emitting dopant including other compounds can be combined for use to allow the light emitting layer to include two or more light emitting dopants. When the light emitting layer includes the compound represented by the general formula (1) above and the polycyclic aromatic compound represented by the general formula (4a), (4b), (5a) or (5b), it is preferable that the compound represented by the general formula (1) above is included as the host material, and the polycyclic aromatic compound material represented by the general formula (4a), (4b), (5a), or (5b) above is included as the light emitting dopant.

The polycyclic aromatic compounds represented by the general formula (4a) or (4b) above as well as the general formula (5a) or (5b) above, can exhibit blue light emission with high efficiency by utilizing the TADF mechanism, however, they have low resistance to holes and electrons, having made it difficult to ensure a practical device lifetime in an organic EL device when used in combination with a conventionally known host material. On the contrary, the compound according to the present invention represented by the general formula (1) above has higher resistance to holes and electrons than these conventionally known compounds, so that when the polycyclic aromatic compound is used as the dopant, the use of the compound of the present invention represented by the general formula (1) above as the host, can result in an organic EL device having a longer lifetime.

When containing two or more light emitting dopants in the light emitting layer, the first dopant is the compound represented by the general formulae (4a), (4b), (5a) and (5b) above or a fluorescent dopant, and for the second dopant, a known compound may be combined for use as the other light emitting dopant. The content of the first dopant is preferably 0.050 to 50% relative to the host materials, and the content of the second dopant is preferably 0.050 to 50% relative to the host materials. The total content of the first dopant and the second dopant do not exceed 50% relative to the host materials.

The other light emitting dopants are known in a large number of patent literatures and the like, and may be selected therefrom. Specific examples of the dopants include, but not limited to, fused ring derivatives such as phenanthrene, anthracene, pyrene, tetracene, pentacene, perylene, naphthopyren, dibenzopyren, rubrene, and chrysene, benzoxazole derivatives, benzothiazole derivatives, benzoimidazole derivatives, benzotriazole derivatives, oxazole derivatives, oxadiazole derivatives, thiazole derivatives, imidazole derivatives, thiadiazole derivatives, triazole derivatives, pyrazoline derivatives, stilbene derivatives, thiophene derivatives, tetraphenylbutadiene derivatives, cyclopentadiene derivatives, bisstyryl derivatives such as bisstyrylanthracene derivatives and distyrylbenzene derivatives, bisstyrylarylene derivatives, diazaindacene derivatives, furan derivatives, benzofuran derivatives, isobenzofuran derivatives, dibenzofuran derivatives, coumarin derivatives, dicyanomethylenopyran derivatives, dicyanomethylenethiopyran derivatives, polymethine derivatives, cyanine derivatives, oxobenzoanthracene derivatives, xanthene derivatives, rhodamine derivatives, fluorescein derivatives, pyrylium derivatives, carbostyryl derivatives, acridine derivatives, oxazine derivatives, phenylene oxide derivatives, quinacridone derivatives, quinazoline derivatives, pyrrolopyridine derivatives, fluoropyridine derivatives, 1,2,5-thiadiazolopyrene derivatives, pyrromethene derivatives, perinone derivatives, pyrrolopyrrole derivatives, squarylium derivatives, violanthrone derivatives, phenazine derivatives, acridone derivatives, deazaflavin derivatives, fluorene derivatives, benzofluorene derivatives, etc.

A phosphorescent dopant can also be used as the other light emitting dopant. The phosphorescent dopant may be a dopant containing an organometallic complex including at least one metal selected from ruthenium, rhodium, palladium, silver, rhenium, osmium, iridium, platinum, and gold. It is more preferably an organometallic complex including platinum, and specifically, the iridium complexes described in J. Am. Chem. Soc. 2001, 123, 4304 and JP2013-530515 A and the platinum complexes described in Adv. Mater. 2014, 26, 7116 and JP2018-2722 A are suitably used, but are not limited thereto.

The phosphorescent dopant material is not particularly limited, but specific examples thereof include the following:

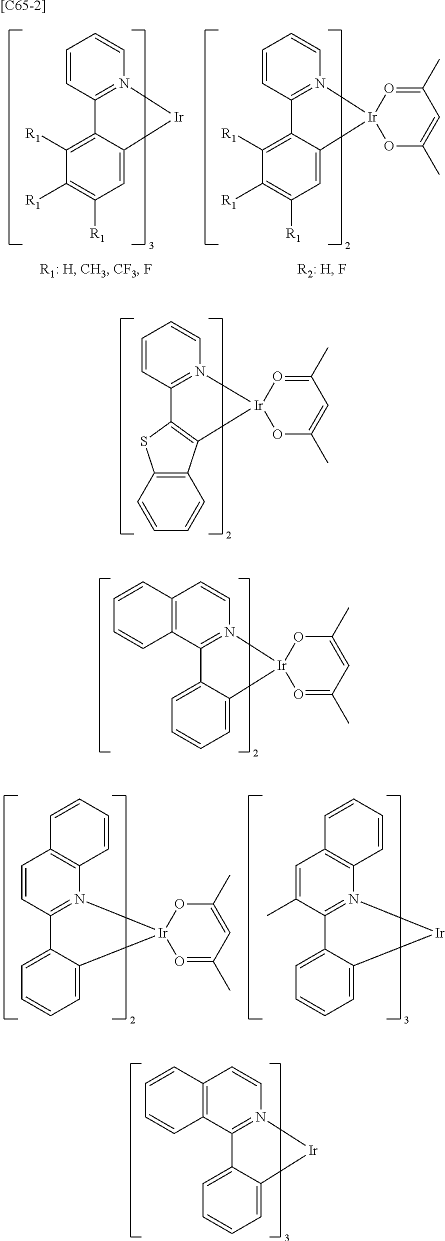

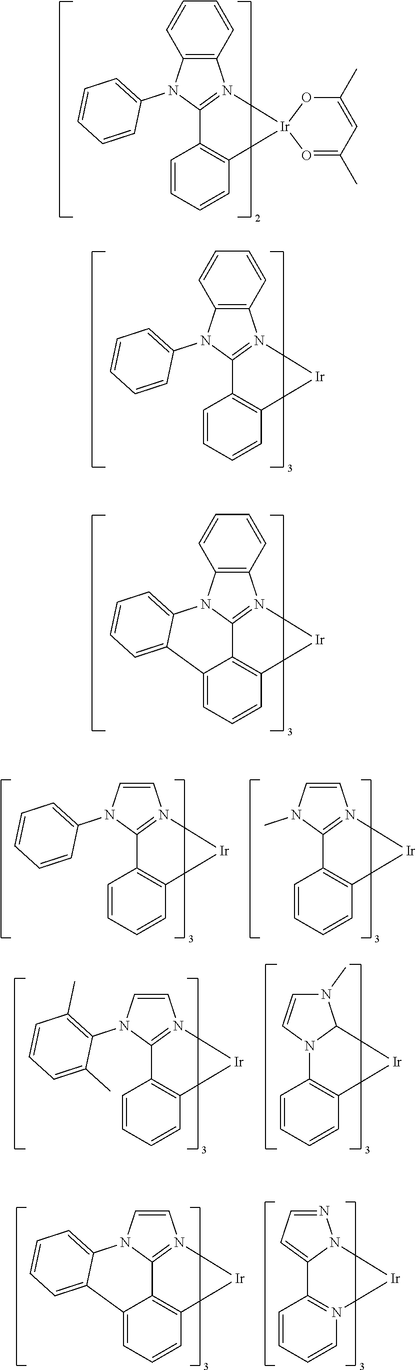

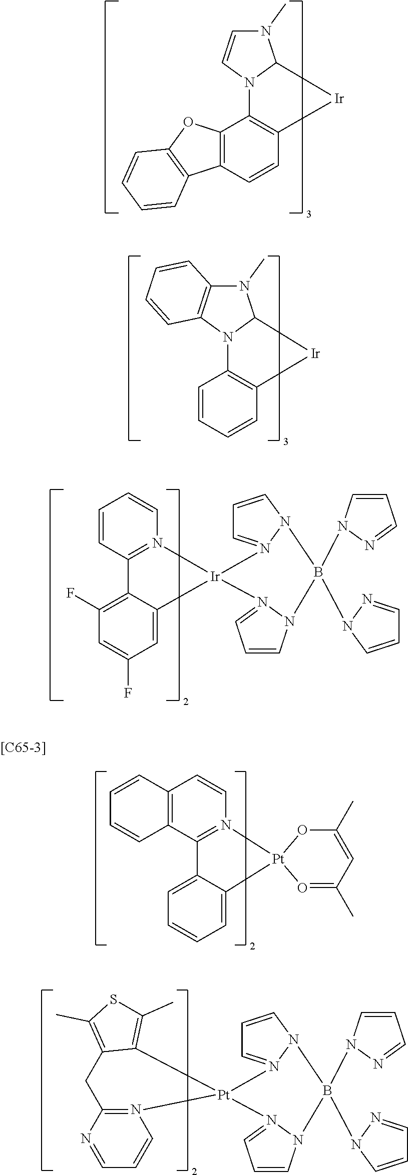

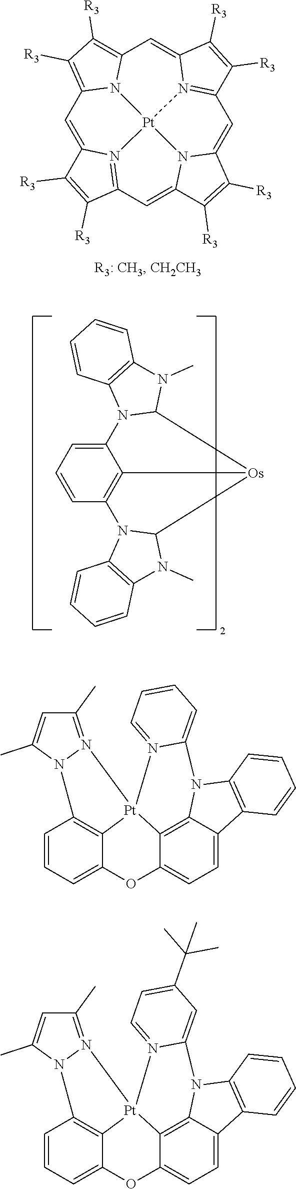

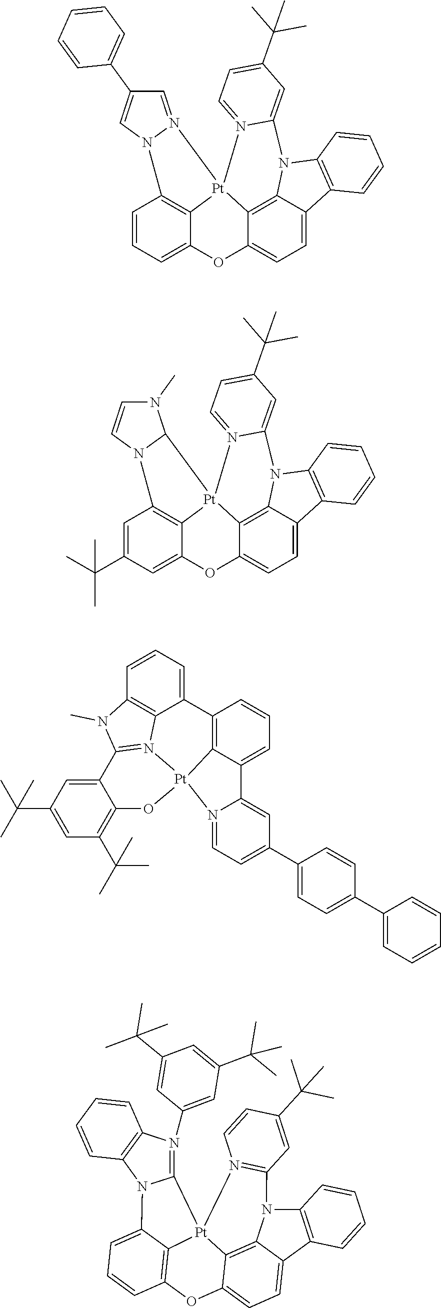

The light emitting dopant and the first or second host can be vapor deposited from different vapor deposition sources, or the light emitting dopant and the first or second host can be premixed before vapor deposition to form a premix, whereby they can be simultaneously deposited from one deposition source.

### —Injection Layer—

The injection layer refers to a layer provided between the electrode and the organic layer to reduce the driving voltage and improve the light emission brightness, and includes the hole injection layer and the electron injection layer. The injection layer may be present between the anode and the light emitting layer or the hole transport layer, as well as between the cathode and the light emitting layer or the electron transport layer. The injection layer may be provided as necessary.

### —Hole Blocking Layer—

The hole blocking layer has the function of the electron transport layer in a broad sense, is made of a hole blocking material having a very small ability to transport holes while having the function of transporting electrons, and can improve the recombination probability between the electrons and the holes in the light emitting layer by blocking the holes while transporting the electrons. For the hole blocking layer, a known hole blocking material can be used. In order to exhibit the characteristics of the light emitting dopant, the material used as the first host can also be used as a material of the hole blocking layer. Also, a plurality of hole blocking materials may be combined for use.

### —Electron Blocking Layer—

The electron blocking layer has the function of the hole transport layer in a broad sense, and can improve the recombination probability between the electrons and the holes in the light emitting layer by blocking the electrons while transporting the holes. As the material for the electron blocking layer, a known material for the electron blocking layer can be used. To exhibit the characteristics of the light emitting dopant, the material used as the second host can also be used as the material for the electron blocking layer. The thickness of the electron blocking layer is preferably 3 to 100 nm, and more preferably 5 to 30 nm.

### —Exciton Blocking Layer—

The exciton blocking layer is a layer to block the diffusion of the excitons generated by recombination of the holes and the electrons in the light emitting layer into a charge transport layer, and insertion of this layer makes it possible to efficiently keep the excitons in the light emitting layer, so that the emission efficiency of the device can be improved. The exciton blocking layer can be inserted between two light emitting layers adjacent to each other in the device in which two or more light emitting layers are adjacent to each other. As the material for the exciton blocking layer, a known material for the exciton blocking layer can be used.

The layer adjacent to the light emitting layer includes the hole blocking layer, the electron blocking layer, and the exciton blocking layer, and when these layers are not provided, the adjacent layer is the hole transport layer, the electron transport layer, and the like.

### —Hole Transport Layer—

The hole transport layer is made of a hole transport material having the function of transporting holes, and the hole transport layer may be provided as a single layer or a plurality of layers.

The hole transport material has any of hole injection properties, hole transport properties, or electron barrier properties, and may be either an organic material or an inorganic material. As the hole transport layer, any of conventionally known compounds may be selected and used. Examples of such a hole transport material include porphyrin derivatives, arylamine derivatives, triazole derivatives, oxadiazole derivatives, imidazole derivatives, polyarylalkane derivatives, phenylenediamine derivatives, arylamine derivatives, amino-substituted chalcone derivatives, oxazole derivatives, styrylanthracene derivatives, fluorenone derivatives, hydrazone derivatives, stilbene derivatives, silazane derivatives, aniline-based copolymers, and conductive polymer oligomers, particularly, thiophene oligomers. Porphyrin derivatives, arylamine derivatives, and styrylamine derivatives are preferably used, and arylamine derivatives are more preferably used.

### —Electron Transport Layer—

The electron transport layer is made of a material having the function of transporting electrons, and the electron transport layer may be provided as a single layer or a plurality of layers.

The electron transport material (may also serve as the hole blocking material) has the function of transmitting electrons injected from the cathode to the light emitting layer. As the electron transport layer, any of conventionally known compounds may be selected and used, and examples thereof include polycyclic aromatic derivatives such as naphthalene, anthracene, and phenanthroline, tris(8-quinolinolato)aluminum (III) derivatives, phosphine oxide derivatives, nitro-substituted fluorene derivatives, diphenylquinone derivatives, thiopyran dioxide derivatives, carbodiimides, fluorenylidene methane derivatives, anthraquinodimethane and anthrone derivatives, bipyridine derivatives, quinoline derivatives, oxadiazole derivatives, benzimidazole derivatives, benzothiazole derivatives, and indolocarbazole derivatives. Further, polymer materials in which these materials are introduced in the polymer chain or these materials constitute the main chain of the polymer can also be used.

When the organic EL device of the present invention is produced, the film formation method of each layer is not particularly limited, and the layers may be produced by either a dry process or a wet process.

### EXAMPLES

The present invention will be described in more detail below by way of Examples, but the present invention is not limited to these Examples and can be implemented in various forms without departing from the gist of the invention.

### Synthesis Example 1

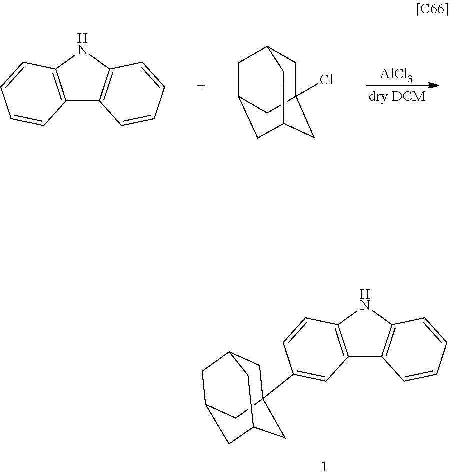

Synthesis of 3-adamantanylcarbazole (1)

A 500 ml Schlenk flask replaced with nitrogen, was added with carbazole (6.02 g, 36.3 mmol) and AlCl₃ (3.12 g, 23.7 mmol) and added with dry dichloromethane (250 mL) under ice bath. After stirring, 1-chloroadamantan (7.44 g, 43.6 mmol) was added and stirred at room temperature for 18 hours. After quenching by addition of with ice water, the resulting mixture was extracted with dichloromethane, and the organic layer was dried with Na₂SO₄ and concentrated. The residue was cleaned with hexane, and the filtrate was then concentrated. The crude product was purified by silica gel column chromatography (eluent: hexane/ethyl acetate=7:1, v/v) and further purified by GPC, yielding a compound 1 [3-adamantanylcarbazole (1)], the target compound. Yield: 19% (2.07 g)

¹H NMR (400 MHz, CDCl₃): δ 8.09-8.05 (m, 2H), 7.95 (s, 1H), 7.48 (dd, J=8.4, 1.4 Hz, 1H), 7.42-7.37 (m, 3H), 7.23-7.20 (m, 1H), 2.15 (s, 3H), 2.05 (s, 6H), 1.82 (s, 6H).

MS (MALDI-TOF): m/z calcd 301.18 [M]⁺; found 301.28.

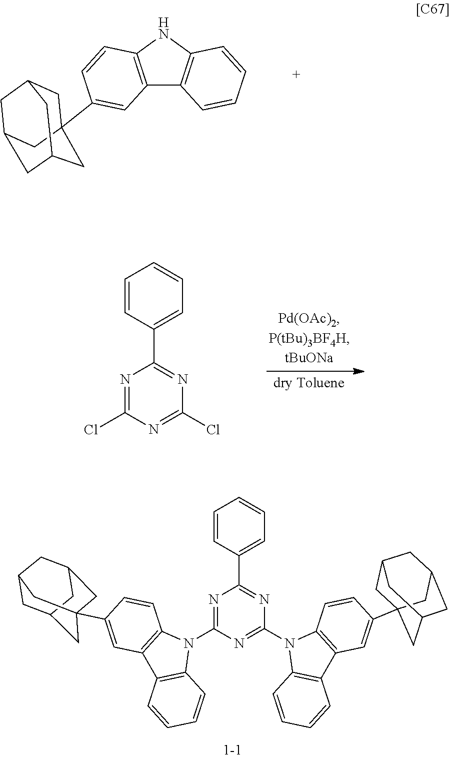 [SMILES: c1ccc2c(c1)[nH]c1ccc(C34CC5CC(CC(C5)C3)C4)cc12 ; Clc1nc(Cl)nc(-c2ccccc2)n1 ; c1ccc(-c2nc(-n3c4ccccc4c4cc(C56CC7CC(CC(C7)C5)C6)ccc43)nc(-n3c4ccccc4c4cc(C56CC7CC(CC(C7)C5)C6)ccc43)n2)cc1]

### Synthesis of Compound 1-1

A 300 ml Schlenk flask replaced with nitrogen, was added with the compound 1 [3-adamantanylcarbazole (2.53 g, 8.38 mmol)] synthesized in the manner described above, 2,4-dichloro-6-phenyl-1.3.5-triazine (663 mg, 2.93 mmol), palladium acetate (69 mg, 0.30 mmol), tri-tert-butylphosphonium tetrafluoroborate (329 mg, 1.13 mmol), t-BuONa (816 mg, 8.49 mmol), and dry toluene (100 mL), and the mixture was stirred at 100° C. for 18 hours. After cooling to room temperature, the resulting liquid was separated with ethyl acetate, and the organic layer was dried with Na₂SO₄ and concentrated. The crude product was purified by silica gel column chromatography (eluent: hexane/dichloromethane=4:1, v/v), yielding a compound 1-1, the target compound. Yield: 57% (1.27 g)

¹H NMR (400 MHz, CDCl₃): δ 9.04 (d, J=8.3 Hz, 2H), 8.97 (d, J=8.8 Hz, 2H), 8.75 (d, J=7.5 Hz, 2H), 8.11 (d, J=7.5 Hz, 2H), 8.07 (s, 2H), 7.67 (m, 3H), 7.60 (d, J=8.8 Hz, 2H), 7.53-7.49 (m, 2H), 7.43 (t, J=7.4 Hz, 2H), 2.19 (s, 6H), 2.10 (s, 12H), 1.85 (s, 12H)

MS (MALDI-TOF): m/z calcd 755.40 [M]⁺; found 756.68.

### Synthesis Example 2

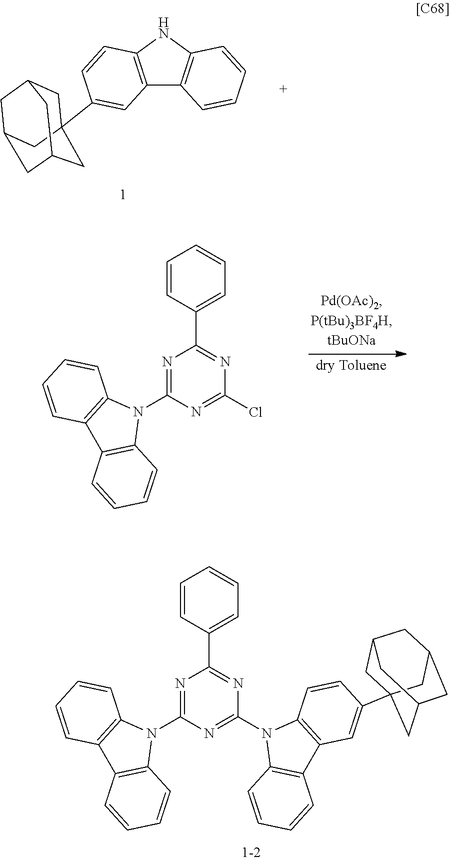 [SMILES: c1ccc2c(c1)[nH]c1ccc(C34CC5CC(CC(C5)C3)C4)cc12 ; Clc1nc(-c2ccccc2)nc(-n2c3ccccc3c3ccccc32)n1 ; c1ccc(-c2nc(-n3c4ccccc4c4ccccc43)nc(-n3c4ccccc4c4cc(C56CC7CC(CC(C7)C5)C6)ccc43)n2)cc1]

### Synthesis of Compound 1-2

A 200 ml Schlenk flask replaced with nitrogen, was added with the compound 1 [3-adamantanyl-carbazole (1.37 g, 4.53 mmol)] synthesized in the manner described above, 9-(4-chloro-6-phenyl-1.3.5-triazin-2-yl)carbazole (1.12 g, 3.14 mmol), palladium acetate (107 mg, 0.48 mmol), tri-tert-butylphosphonium tetrafluoroborate (377 mg, 1.3 mmol), t-BuONa (607 mg, 6.3 mmol), and dry toluene (90 mL), and the mixture was stirred at 90° C. for 17 hours. After cooling to room temperature, the resulting liquid was separated with ethyl acetate, and the organic layer was dried with Na₂SO₄ and concentrated. The crude product was purified by silica gel column chromatography (eluent: hexane/dichloromethane=4:1, v/v), yielding a compound 1-2, the target compound. Yield: 62% (1.21 g)

¹H NMR (400 MHz, CDCl₃): δ 9.07-9.03 (m, 3H), 8.98 (d, J=8.8 Hz, 1H), 8.75 (dd, J=7.7, 1.9 Hz, 2H), 8.11 (d, J=7.5 Hz, 3H), 8.07 (d, J=1.5 Hz, 1H), 7.68 (d, J=6.8 Hz, 3H), 7.60-7.49 (m, 4H), 7.44 (q, J=7.0 Hz, 3H), 2.18 (s, 3H), 2.10 (s, 6H), 1.85 (s, 6H)

MS (MALDI-TOF): m/z calcd 621.29 [M]⁺; found 621.45.

The compounds used in Examples and Comparative Examples will be shown below.

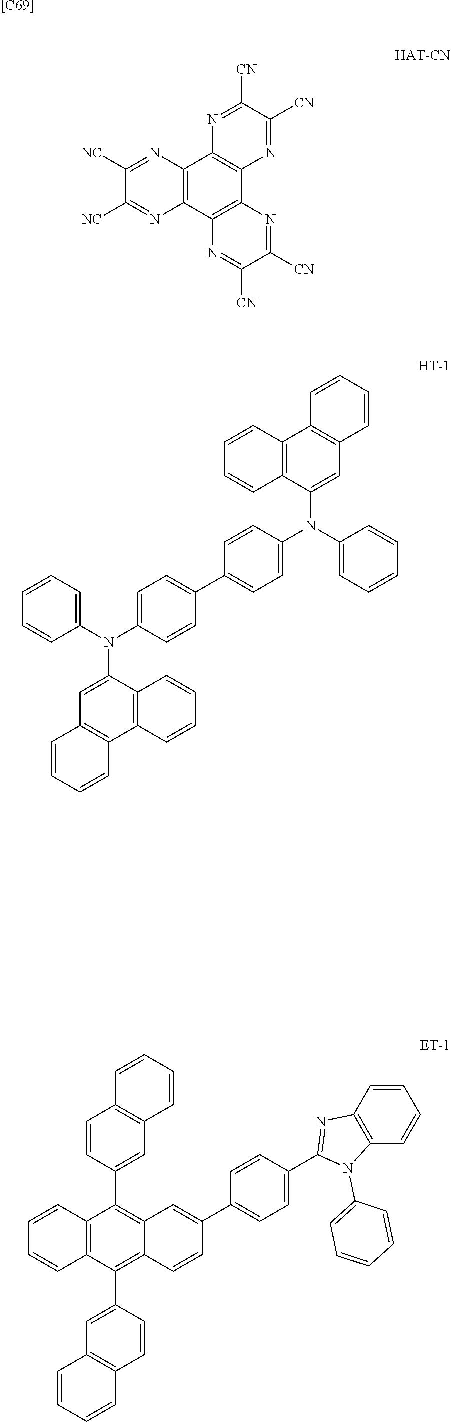

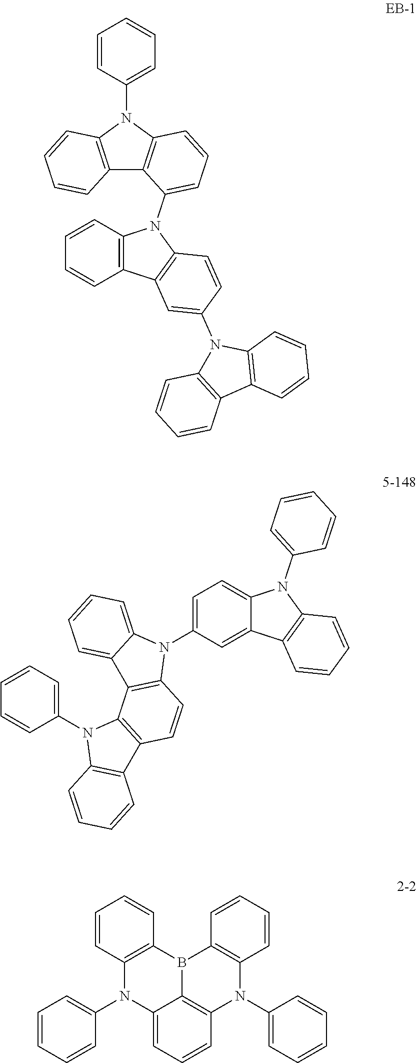 [SMILES: c1ccc(-n2c3ccccc3c3c(-n4c5ccccc5c5cc(-n6c7ccccc7c7ccccc76)ccc54)cccc32)cc1 ; c1ccc(-n2c3ccccc3c3cc(-n4c5ccccc5c5c4ccc4c6ccccc6n(-c6ccccc6)c45)ccc32)cc1 ; c1ccc(N2c3ccccc3B3c4ccccc4N(c4ccccc4)c4cccc2c43)cc1]

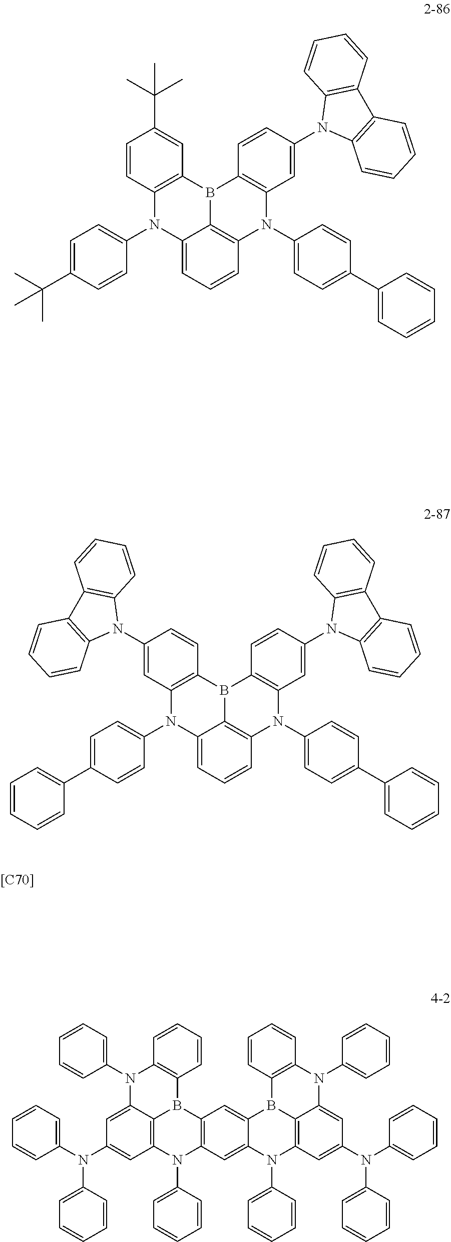

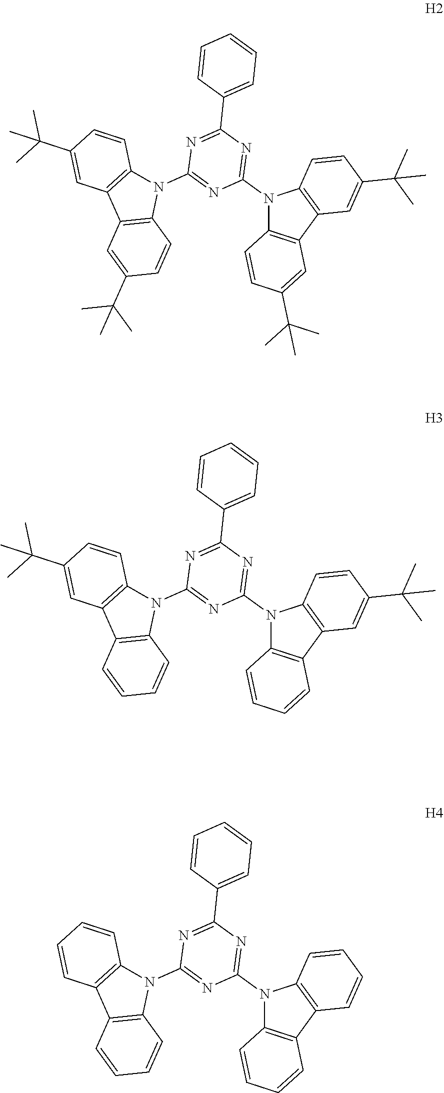

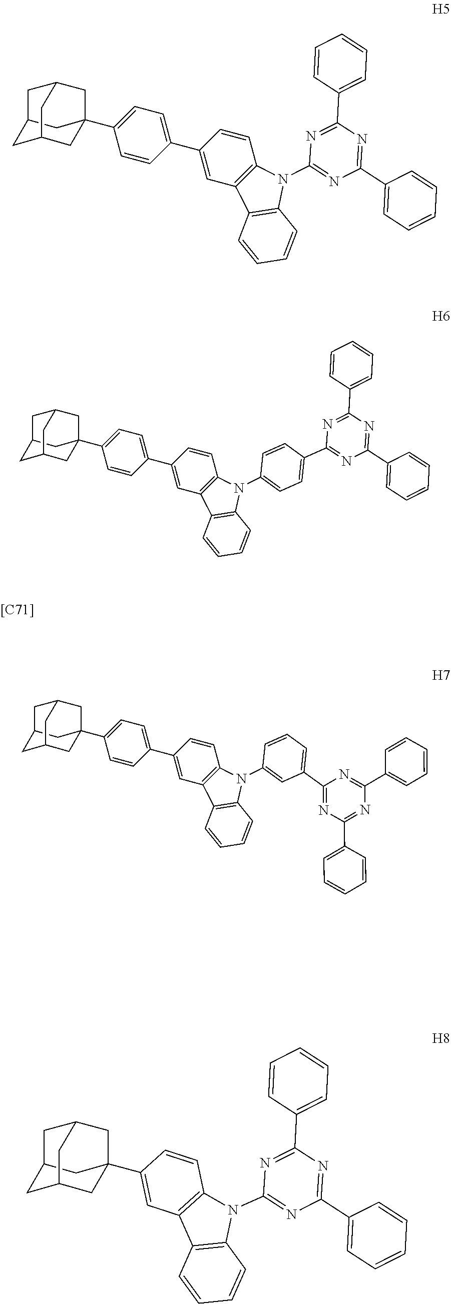

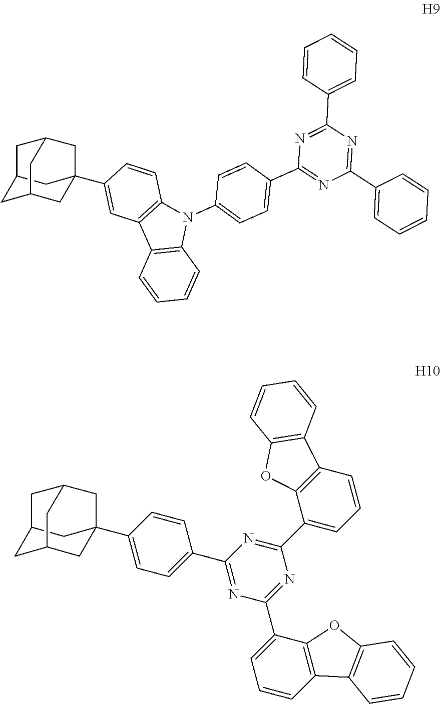 [SMILES: c1ccc(-c2nc(-c3ccccc3)nc(-c3ccc(-n4c5ccccc5c5cc(C67CC8CC(CC(C8)C6)C7)ccc54)cc3)n2)cc1 ; c1ccc2c(c1)oc1c(-c3nc(-c4ccc(C56CC7CC(CC(C7)C5)C6)cc4)nc(-c4cccc5c4oc4ccccc45)n3)cccc12]

### CALCULATION EXAMPLE

### Calculation of HOMO and LUMO Values

HOMO and LUMO were calculated for the above compounds 1-1, 1-2, 1-3, 1-4, 1-5, 1-6, 1-7, and 1-8. The calculation was performed using the density functional theory (DFT), Gaussian as a calculation program, and structural optimization calculation by density functional calculation B3LYP/6-31G(d). The results are shown in Table 1 below. All of the materials of the present invention represented by the general formula (1) can be said to have preferable HOMO and LUMO values as host materials.

TABLE 1

| Compound | HOMO(eV) | LUMO(eV) |
|---|---|---|
| 1-1 | −5.6 | −1.7 |
| 1-2 | −5.7 | −1.8 |
| 1-3 | −5.8 | −1.8 |
| 1-4 | −5.8 | −1.4 |
| 1-5 | −5.7 | −1.7 |
| 1-6 | −5.3 | −1.8 |
| 1-7 | −5.2 | −1.9 |
| 1-8 | −5.8 | −1.8 |
| H2 | −5.6 | −1.7 |
| H3 | −5.7 | −1.8 |
| H4 | −5.8 | −1.8 |
| H5 | −5.5 | −1.9 |
| H6 | −5.2 | −1.9 |
| H7 | −5.2 | −2.0 |
| H8 | −5.6 | −1.9 |
| H9 | −5.3 | −1.9 |
| H10 | −6.0 | −1.8 |

### Measurement of Tg Value

A glass transition temperature Tg was measured for the above-described compounds 1-1 and 1-2. It is to be noted that a DSC7020 manufactured by Hitachi High-Tech Corporation was used. The results are shown in Table 2 below. All of the compounds of the present invention represented by formula (1) above can be said to have a preferable Tg value.

TABLE 2

| Compound | Tg (° C.) |
|---|---|
| 1-1 | 200° C. |
| 1-2 | 145° C. |
| H3 | 111° C. |
| H4 | 95° C. |

S1 and T1 of the above-described compounds 2-2 and 4-2 were measured by the following method.

Powder of the compound 2-2 or compound 4-2 was dissolved in toluene solvent, and its solution was prepared to a concentration of 10⁻⁵ M.

For S1, the emission spectrum of this solution was measured, a tangent was drawn to the rise of the emission spectrum on the short-wavelength side, and the wavelength value λedge [nm] of the point of intersection of the tangent and the horizontal axis was substituted into the following equation (i) to calculate S1.

$$S 1 [ eV ] = 1239.85 / λ edge \qquad ( i )$$

For T1, the phosphorescence spectrum of the above solution was measured, a tangent was drawn to the rise of the phosphorescence spectrum on the short-wavelength side, and the wavelength value λedge [nm] of the point of intersection of the tangent and the horizontal axis was substituted into the following equation (ii) to calculate T1.

$$T 1 [ eV ] = 1239.85 / λ edge \qquad ( ii )$$

The measurement results of S1 and T1, and the value of ΔEST, which is a difference between S1 and T1, are shown in Table 3.

TABLE 3

| Compound | S1(eV) | T1(eV) | ΔEST(eV) |
|---|---|---|---|
| 2-2 | 2.79 | 2.61 | 0.18 |
| 4-2 | 2.71 | 2.67 | 0.04 |

The compounds 2-2 and 4-2 indicating a ΔEST of 0.2 eV or less, which can be said to be generally suitable for a thermally activated delayed fluorescence material, facilitates reverse intersystem crossing to occur, making it possible to efficiently use triplet excitons for light emission, so that high emission efficiency can be expected.

S1 and T1 can also be determined by the actual measurement as described above, or also by theoretical calculation using a molecular orbital program as will be described below. It is to be noted that the absolute value of ΔEST(theo) obtained by the following calculation method differs from that of ΔEST actually measured, however, in general, the smaller the value is, the more reverse intersystem crossing is likely to occur, making it possible to efficiently use triplet excitons for light emission, so that high emission efficiency can be expected. Also, a thermally activated delayed fluorescence material with a small ΔEST(theo) also generally has a small ΔEST actually measured.

For the light emitting materials 2-2, 4-2, 2-86, and 2-87 represented by the general formula (4a) or (4b), molecular orbital program Gaussian 16 was used to perform structural optimization calculation at the TDA-PBE0/6-31G* level based on a density function theory (DFT), and S1(theo), T1(theo), and the ΔEST(theo) were calculated. The results are shown in Table 4.

TABLE 4

| | S1(theo) | T1(theo) | ΔEST(theo) |
|---|---|---|---|
| Compound | [eV] | [eV] | [eV] |
| 2-2 | 3.22 | 2.67 | 0.55 |
| 4-2 | 2.98 | 2.62 | 0.36 |
| 2-86 | 3.09 | 2.62 | 0.47 |
| 2-87 | 3.05 | 2.60 | 0.45 |

It is found in Table 4 that the compound 2-2 according to Example indicting a ΔEST of 0.2 eV or less, which can be said to be generally suitable for an actual measurement value, indicates a ΔEST(theo) [eV] of 0.60 eV or less, which is a theoretical calculation value.

As shown in Table 4, 4-13, 2-86, and 2-87, which are the light emitting materials represented by the general formula (4a) or (4b), indicate the same small ΔEST (theo) as 2-2 and 4-2 having a small ΔEST value actually measured and therefore facilitate reverse intersystem crossing to occur, making it possible to efficiently use triplet excitons for light emission, so that high emission efficiency can be expected.

### Example 1

Each thin film was laminated on the glass substrate on which an anode made of ITO having a film thickness of 70 nm was formed by a vacuum deposition method at a degree of vacuum of 4.0×10⁻⁵ Pa. First, HAT-CN was formed on ITO to a thickness of 10 nm as a hole injection layer, and then HT-1 was formed to a thickness of 25 nm as a hole transport layer. Then, the compound EB-1 was formed to a thickness of 5 nm as an electron blocking layer. Then, the compound 1-1 as the first host, a compound 5-148 as the second host, and the compound 4-2 as the light emitting dopant were vapor co-deposited from different vapor deposition sources to form a light emitting layer to a thickness of 30 nm. At this time, they were vapor co-deposited under the vapor deposition conditions such that the concentration of the compound 4-2 was 2% and the mass ratio of the first host to the second host was 30:70. Then, compound H1 was formed to a thickness of 5 nm as a hole blocking layer. Then, ET-1 was formed to a thickness of 40 nm as an electron transport layer. Lithium fluoride (LiF) was formed on the electron transport layer to a thickness of 1 nm as an electron injection layer. Finally, aluminum (Al) was formed on the electron injection layer to a thickness of 70 nm as a cathode, whereby an organic EL device was produced.

### Examples 2 to 5, and Comparative Example 1

Each organic EL device was produced in the same manner as in Example 1, except that the light emitting dopant, first host, and second host as well as the mass ratio between the first host and the second host were changed to the compounds, or the mass ratio as shown in Table 3. The mass ratio denotes the first host:the second host.

TABLE 5

| | Light emitting | First | Second | Mass |
|---|---|---|---|---|
| | dopant | host | host | ratio |
| Example 1 | 4-2 | 1-1 | 5-148 | 50:50 |
| Example 2 | 4-2 | 1-2 | 5-148 | 50:50 |
| Example 3 | 4-2 | 1-2 | 5-148 | 40:60 |
| Example 4 | 4-2 | 1-2 | 5-148 | 30:70 |
| Example 5 | 4-2 | 1-2 | 5-148 | 20:80 |
| Comparative | 4-2 | H2 | 5-148 | 50:50 |
| Example 1 | | | | |

Table 6 shows the maximum emission wavelength of the light emission spectrum, the external quantum efficiency, and the device lifetime of each organic EL device produced in Examples and Comparative Examples. The maximum emission wavelength and the external quantum efficiency were values at a current density of 2.5 mA/cm² and were initial properties. As the device lifetime, the time taken for the luminance to reduce to 70% of the initial luminance at a current density of 2.5 mA/cm² was measured.

TABLE 6

| | Maximum | | | External | |
|---|---|---|---|---|---|
| | emission | | | quantum | Device |
| | wavelength | | Voltage | efficiency | lifetime |
| | (nm) | CIEy | (V) | (%) | (h) |
| Example 1 | 470 | 0.14 | 3.6 | 24 | 35 |
| Example 2 | 471 | 0.15 | 3.5 | 24 | 36 |
| Example 3 | 471 | 0.15 | 3.6 | 24 | 41 |
| Example 4 | 471 | 0.14 | 3.6 | 23 | 44 |
| Example 5 | 471 | 0.14 | 3.6 | 21 | 37 |
| Comparative | 471 | 0.13 | 3.7 | 22 | 29 |
| Example 1 | | | | | |

It is found from the maximum emission wavelengths in Table 6 that the organic EL devices of Examples 1 to 5 and Comparative Example 1 exhibit blue light emission. Also, it is found from the results in Table 6 that Examples 1 to 5 have improved external quantum efficiency or device lifetime compared to Comparative Examples, and have high efficiency and long lifetime characteristics as organic EL devices exhibiting blue light emission. In other words, the compound of the present application represented by the general formula (1) is found to have superior characteristics compared to the known compounds of Comparative Examples.

The compounds used in Example 6 and Comparative Example 2 are shown below.

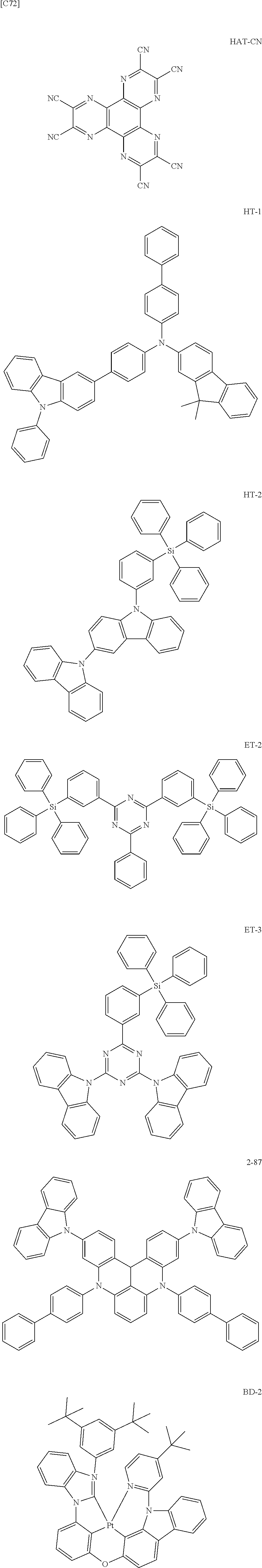

### Example 6

Each thin film was laminated on the glass substrate on which an anode made of ITO having a film thickness of 70 nm was formed by a vacuum deposition method at a degree of vacuum of 4.0×10⁻⁵ Pa. First, the above-described HAT-CN was formed on ITO to a thickness of 10 nm as a hole injection layer, and then HT-1 was formed to a thickness of 60 nm as a hole transport layer. Then, HT-2 was formed to a thickness of 5 nm as an electron blocking layer. Then, the compound (1-4-p) as the first host, the compound HT-2 as the second host, the compound BD-2 that was a phosphorescent dopant as the second dopant, and the compound 2-87 as the first dopant were vapor co-deposited from different vapor deposition sources to form a light emitting layer to a thickness of 40 nm. At this time, they were vapor co-deposited under the vapor deposition conditions such that the concentration of BD-2 was 13% by mass, the concentration of 2-87 was 0.4% by mass, and the mass ratio of the first host to the second host was 40:60. Then, ET-2 was formed to a thickness of 5 nm as a hole blocking layer. Then, ET-2 was formed to a thickness of 31 nm as an electron transport layer. Further, lithium fluoride (LiF) was formed on the electron transport layer to a thickness of 1 nm as an electron injection layer. Finally, aluminum (Al) was formed on the electron injection layer to a thickness of 70 nm as a cathode, whereby the organic EL device according to Example 6 was produced.

### Comparative Example 2

An organic EL device was produced in the same manner as in Example 6, except that the first host and the second host were the compounds shown in Table 7.

TABLE 7

| | | | Mixing ratio (first |
|---|---|---|---|
| | First host | Second host | host:second host) |
| Example 6 | 1-4-p | HT-2 | 40:60 |
| Comparative | ET-3 | HT-2 | 40:60 |
| Example 2 | | | |

The evaluation results of the organic EL devices produced are shown in Table 2. When an external power source was connected to the organic EL devices obtained in Examples and Comparative Examples and a direct current voltage was applied thereto, an emission spectrum with the maximum emission wavelength of 450 nm to 480 nm was observed in all organic EL devices, and it was found that the compound 2-87 exhibited light emission.

The voltage and power efficiency in the table are the values when a driving current was 4.0 mA/cm², and are initial properties. Also, the lifetime is the time taken for the luminance to reduce to 95% when the initial luminance was 100% at a driving current of 4.0 mA/cm² and represents device lifetime characteristics. Furthermore, the emission color was confirmed by the emission spectrum of the organic EL device.

It is found from the results of Examples and Comparative Examples in Table 8 that the organic EL devices using the mixed material for an organic electroluminescent device of the present invention as the host, in the light emitting layer exhibit blue light emission and have long lifetime characteristics.

TABLE 8

| | Light | | Power | |
|---|---|---|---|---|
| | emission | Voltage | efficiency | Lifetime |
| | color | (V) | (lm/W) | (h) |
| Example 6 | Blue | 4.3 | 18.3 | 15 |
| Comparative | Blue | 4.2 | 18.7 | 11 |
| Example 2 | | | | |

### REFERENCE SIGNS LIST

1 substrate, 2 anode, 3 hole injection layer, 4 hole transport layer, 5 light emitting layer, 6 electron transport layer, 7 cathode

1. A compound for an organic electroluminescent device, represented by the following general formula (1): 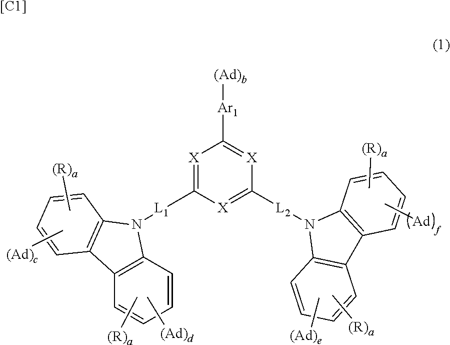
wherein Ad is an adamantyl group represented by the following general formula (2): 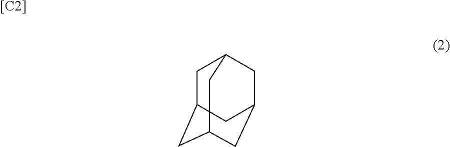
wherein X independently represents N or CR¹, and at least one X represents N; R¹ independently represents hydrogen, deuterium, an aliphatic hydrocarbon group having 1 to 10 carbon atoms, a substituted or unsubstituted aromatic hydrocarbon group having 6 to 18 carbon atoms, a substituted or unsubstituted aromatic heterocyclic group having 3 to 17 carbon atoms, or a substituted or unsubstituted linked aromatic group formed by linking 2 to 8 of these aromatic groups;
Ar¹ represents a substituted or unsubstituted aromatic hydrocarbon group having 6 to 18 carbon atoms, a substituted or unsubstituted aromatic heterocyclic group having 3 to 17 carbon atoms, or a substituted or unsubstituted linked aromatic group formed by linking 2 to 8 of these aromatic groups;
R independently represents deuterium, an aliphatic hydrocarbon group having 1 to 10 carbon atoms, a substituted or unsubstituted aromatic hydrocarbon group having 6 to 18 carbon atoms, or a substituted or unsubstituted aromatic heterocyclic group having 3 to 17 carbon atoms;
L¹ and L² each independently represent a direct bond, a substituted or unsubstituted aromatic hydrocarbon group having 6 to 18 carbon atoms, a substituted or unsubstituted aromatic heterocyclic group having 3 to 17 carbon atoms, or a substituted or unsubstituted linked aromatic group formed by linking 2 to 8 of these aromatic groups;
a represents the number of substitutions and independently represents an integer of 0 to 4; and b to f represent the number of substitutions and independently represent an integer of 0 to 4, provided that b+c+d+e+f≥1 is satisfied.

2. The compound for an organic electroluminescent device according to claim 1, wherein each X is represented by N.

3. The compound for an organic electroluminescent device according to claim 1, wherein b+c+d≥1 is satisfied.

4. The compound for an organic electroluminescence device according to claim 1, wherein b+c+d+e+f≥2 is satisfied.

5. The compound for an organic electroluminescence device according to claim 1, wherein Ar¹ is represented by a substituted or unsubstituted aromatic hydrocarbon group having 6 to 11 carbon atoms, a substituted or unsubstituted aromatic heterocyclic group having 3 to 11 carbon atoms, or a substituted or unsubstituted linked aromatic group formed by linking 2 to 8 of these aromatic groups.

6. The compound for an organic electroluminescence device according to claim 1, wherein each a is represented by 0.

7. The compound for an organic electroluminescence device according to claim 1, wherein L¹ and L² are independently represented by a direct bond, a substituted or unsubstituted aromatic hydrocarbon group having 6 to 18 carbon atoms, or a substituted or unsubstituted aromatic heterocyclic group having 3 to 17 carbon atoms.

8. The compound for an organic electroluminescence device according to claim 1, wherein Ad is an adamantyl group represented by the following general formula (3): 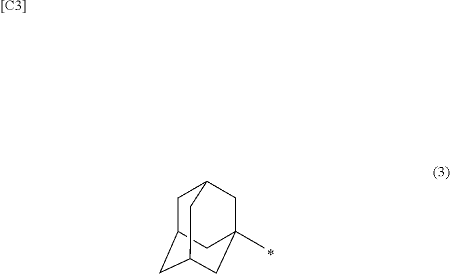
wherein “*” is a bonding site to the general formula (1) above.

9. The compound for an organic electroluminescence device according to claim 1, wherein the compound has a glass transition temperature (Tg) of 135° C. or higher.

10. An organic electroluminescence device, comprising one or more light emitting layers between an anode and a cathode opposite to each other, wherein at least one of the light emitting layers comprises a host selected from the compounds represented by the general formula (1) described in claim 1, and a light emitting dopant.

11. The organic electroluminescence device according to claim 10, comprising, as the light emitting dopant, a light emitting dopant containing a boron atom.

12. The organic electroluminescence device according to claim 10, comprising, as the light emitting dopant, a polycyclic aromatic compound represented by the following general formula (4a) or (4b): 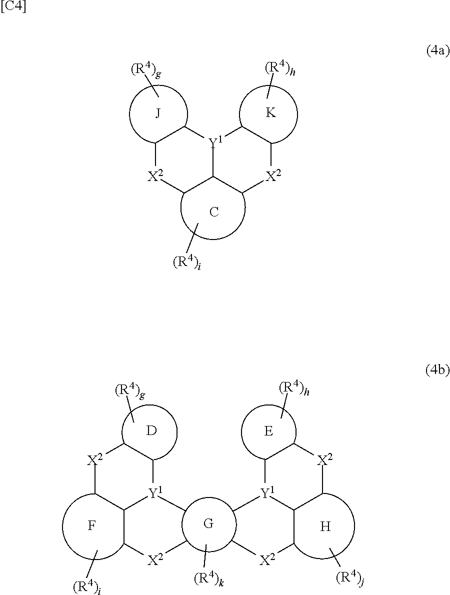
wherein, a ring J, a ring K, a ring C, a ring D, a ring E, a ring F, a ring G, and a ring H are each independently a substituted or unsubstituted aromatic hydrocarbon ring having 6 to 24 carbon atoms, or a substituted or unsubstituted aromatic heterocyclic ring having 3 to 17 carbon atoms;
Y¹ is each independently B, P, P═O, P═S, Al, Ga, As, Si—R³, or Ge—R³;
R³ is each independently an aliphatic hydrocarbon group having 1 to 10 carbon atoms, a substituted or unsubstituted aromatic hydrocarbon group having 6 to 18 carbon atoms, or a substituted or an unsubstituted aromatic heterocyclic group having 3 to 17 carbon atoms;
X² is each independently O, N—Ar⁴, S, or Se;
Ar⁴ is each independently a substituted or unsubstituted aromatic hydrocarbon group having 6 to 18 carbon atoms, a substituted or unsubstituted aromatic heterocyclic group having 3 to 17 carbon atoms, or a substituted or unsubstituted linked aromatic group formed by linking 2 to 8 of these groups, and N—Ar⁴ is optionally bonded to any of the ring J, the ring K, the ring C, the ring D, the ring E, the ring F, the ring G, or the ring H to form a heterocyclic ring containing N;
R⁴ each independently represents a cyano group, deuterium, a diarylamino group having 12 to 44 carbon atoms, an arylheteroarylamino group having 12 to 44 carbon atoms, a diheteroarylamino group having 12 to 44 carbon atoms, an aliphatic hydrocarbon group having 1 to 10 carbon atoms, a substituted or unsubstituted aromatic hydrocarbon group having 6 to 18 carbon atoms, or a substituted or unsubstituted aromatic heterocyclic group having 3 to 17 carbon atoms;
g and h represent the number of substitutions and each independently represent an integer of 0 to 4, i and j represent the number of substitutions and each represent an integer of 0 to 3, and k represents the number of substitutions and represents an integer of 0 to 2.

13. The organic electroluminescent device according to claim 12, wherein the general formula (4a) or (4b) is a polycyclic aromatic compound represented by the following general formula (5a) or (5b), respectively: 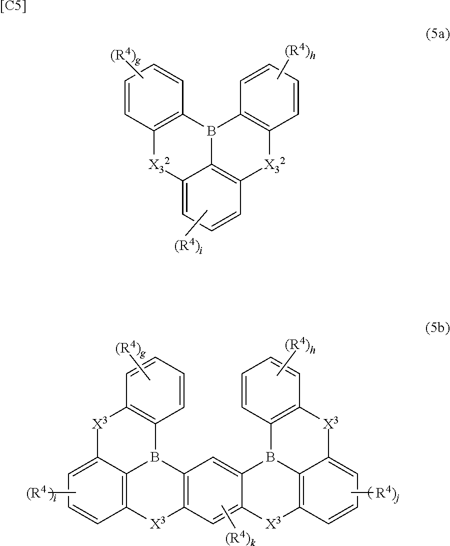
wherein X³ each independently represents N—Ar⁴, O, or S, and at least one X³ represents N—Ar⁴; and Ar⁴, R⁴, g, h, i, j, and k are as defined for the general formula (4a) or (4b).

14. The organic electroluminescent device according to claim 13, wherein the polycyclic aromatic compounds represented by the general formulae (4a), (4b), (5a), and (5b) have a difference between excited singlet energy (S1) and excited triplet energy (T1) (ΔEST) of 0.20 eV or less.

15. The organic electroluminescence device according to claim 10, wherein in the organic electroluminescence device comprising one or more light emitting layers between an anode and a cathode opposite to each other, at least one of the light emitting layers comprises a first host selected from the compounds represented by the general formula (1), a second host, and a light emitting dopant containing a boron atom.

16. The organic electroluminescence device according to claim 15, wherein the second host is selected from compounds represented by the following general formula (6): 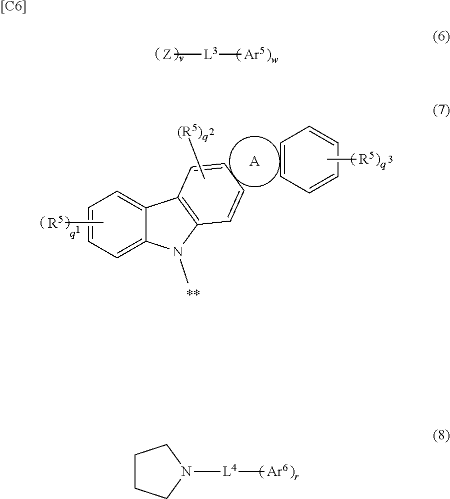
wherein Z is an indolocarbazole ring-containing group represented by general formula (7), and “**” represents a bonding site to L³; and the ring A in the general formula (7) is a heterocyclic ring represented by the general formula (8), and the ring A is condensed with the adjacent ring at any position;
L³ and L⁴ are each independently a substituted or unsubstituted aromatic hydrocarbon group having 6 to 18 carbon atoms or a substituted or unsubstituted aromatic heterocyclic group having 3 to 17 carbon atoms;
Ar⁵ and Ar⁶ are each independently deuterium, a substituted or unsubstituted aromatic hydrocarbon group having 6 to 18 carbon atoms, a substituted or unsubstituted aromatic heterocyclic group having 3 to 17 carbon atoms, or a substituted or unsubstituted linked aromatic group formed by linking 2 to 8 of these aromatic groups;
R⁵ is independently deuterium, an aliphatic hydrocarbon group having 1 to 10 carbon atoms, a substituted or unsubstituted aromatic hydrocarbon group having 6 to 18 carbon atoms, or a substituted or unsubstituted aromatic heterocyclic group having 3 to 17 carbon atoms;
v represents the number of substitutions and represents an integer of 1 to 3, w represents the number of substitutions and represents an integer of 0 to 3, q¹ and q³ represent the number of substitutions and each independently represent an integer of 0 to 4, q² represents the number of substitutions and represents an integer of 0 to 2, and r represents the number of substitutions and represents an integer of 0 to 3.

17. The organic electroluminescent device according to claim 16, wherein the general formula (6) is a compound represented by the following general formula (6a) or (6b): 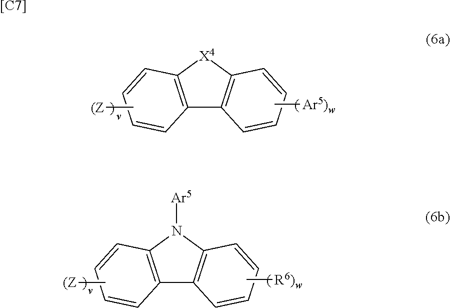
wherein Z, Ar⁵, v, and w are as defined for the general formula (6), and X⁴ represents O or S; and R⁶ is each independently deuterium, an aliphatic hydrocarbon group having 1 to 10 carbon atoms, a substituted or unsubstituted aromatic hydrocarbon group having 6 to 18 carbon atoms, or a substituted or unsubstituted aromatic heterocyclic group having 3 to 17 carbon atoms.
